SEQUENCE LISTING (15 sequences)

SEQ ID NO: 1 - RNA, 87 nt, Homo sapiens
ucagagugagguaguagauuguauaguugugggguagugauuuuacccuguucaggagauaacuauacaaucuauugccuucccuga

SEQ ID NO: 2 - RNA, 87 nt, Artificial Sequence
  Mutation from hsa0000063
ucagagugaggcaguagauuguauaguugugggguagugauuuuacccuguucaggagauaacuauacaaucuauugccuucccuga

SEQ ID NO: 3 - RNA, 87 nt, Artificial Sequence
  Mutation from hsa0000063
ucagagugaggcaguagauuguauaguugugggguagugauuuuacccuguucaggagacaacuauacaaucuauugccuucccuga

SEQ ID NO: 4 - RNA, 87 nt, Artificial Sequence
  Mutation from hsa0000063
ucagagugaggcaguagauuguauaguugugggguagugauuuuacccuguucaggagacaacuauacaaucuacugccuucccuga

SEQ ID NO: 5 - RNA, 87 nt, Artificial Sequence
  Mutation from hsa0000063
ucagagugaggcaguagauuguauaguugugggguagugauucuacccuguucaggagacaacuauacaaucuacugccuucccuga

SEQ ID NO: 6 - RNA, 87 nt, Artificial Sequence
  Mutation from hsa0000063
ucagagugaggcaguagauuguauaguugugggguagugauucuaccccguucaggagacaacuauacaaucuacugccuucccuga

SEQ ID NO: 7 - RNA, 87 nt, Artificial Sequence
  Mutation from hsa0000063
ucagagugaggcaguagauuguauaguugugggguagugauucuaccccguucaggagacaacuauacaaucuacugccuugccuga

SEQ ID NO: 8 - RNA, 87 nt, Artificial Sequence
  Mutation from hsa0000063
ucagagugaggcaguagauugcauaguugugggguagugauucuaccccguucaggagacaacuaugcaaucuacugccuugccuga

SEQ ID NO: 9 - RNA, 87 nt, Artificial Sequence
  Mutation from hsa0000063
ucagagugaggcaguagauugcauaguugugggguagugauucuaccccguucaggagacagcuaugcaaucuacugccuugccuga

SEQ ID NO: 10 - RNA, 87 nt, Artificial Sequence
  Mutation from hsa0000063
ucagagugaggcaguagauugcauagcugugggguagugauucuaccccguucaggagacagcuaugcaaucuacugccuugccuga

SEQ ID NO: 11 - RNA, 87 nt, Artificial Sequence
  Mutation from hsa0000063
ucagagugaggcaguagauugcauagccgugggguagugauucuaccccguucaggagacggcuaugcaaucuacugccuugccuga

SEQ ID NO: 12 - RNA, 87 nt, Artificial Sequence
  Mutation from hsa0000063
ucagaguggggcaguagauugcauagccgugggguagugauucuaccccguucaggagacggcuaugcaaucuacugcccugccuga

SEQ ID NO: 13 - RNA, 87 nt, Artificial Sequence
  Mutation from hsa0000063
ucagaguggggcaguagguugcauagccgugggguagugauucuaccccguucaggagacggcuaugcaaccuacugcccugccuga

SEQ ID NO: 14 - RNA, 87 nt, Artificial Sequence
  Mutation from hsa0000063
ucagaguggggcaguaggucgcauagccgugggguagugauucuaccccguucaggagacggcuaugcgaccuacugcccugccuga

SEQ ID NO: 15 - RNA, 87 nt, Artificial Sequence
  Mutation from hsa0000063
ucagaguggggcaguaggucgcacagccgugggguagugauucuaccccguucaggagacggcugugcgaccuacugcccugccuga

(19) United States
(12) Patent Application Publication
(10) Pub. No.: US 2019/0147974 A1
(43) Pub. Date: May 16, 2019

(54) Systems And Methods For Characterizing And Sampling Nucleic Acid Sequences And Structures Of Same

(71) Applicant: Virginia Polytechnic Institute and State Universit, Blacksburg, VA (US)

(72) Inventors: Christopher L. Barrett, Blacksburg, VA (US); Christian Reidys, Blacksburg, VA (US); Qijun He, Blacksburg, VA (US); Wenda Huang, Blacksburg, VA (US)

(73) Assignee: Virginia Polytechnic Institute and State Universit, Blacksburg, VA (US)

(21) Appl. No.: 16/158,964

(22) Filed: Oct. 12, 2018

Related U.S. Application Data
(60) Provisional application No. 62/571,840, filed on Oct. 13, 2017.

(51) Int. Cl. G16B 15/10; C12Q 1/6869; G16B 30/10

(52) U.S. Cl. CPC G16B 15/10; G16B 30/10; C12Q 1/6869

(57) ABSTRACT

Methods and systems for comparing nucleic acid sequences together with their associated structural forms are described. The methods and systems can be used for comparing, identifying, and characterizing sets of nucleic acid sequences that meet a certain set of criteria, including structural similarity and/or nucleotide sequence similarity. The methods and systems can also be used for identifying nucleic acid sequences that form compatible structures and/or identifying nucleic acid structures that can arise from the same or substantially similar nucleotide sequences.

### CROSS REFERENCE TO RELATED PATENT APPLICATIONS

This application claims the benefit of U.S. Provisional Patent Application No. 62/571,840, filed on Oct. 13, 2017, which is hereby incorporated by reference in its/their entirety.

### SEQUENCE LISTING INFORMATION

A computer readable text file, entitled “Sequence-listing.txt,” created on or about Dec. 18, 2018, with a file size of about 4 KB, contains the sequence listing for this application and is hereby incorporated by reference in its entirety.

### FIELD

The present disclosure is directed to systems and methods for sampling, identifying, characterizing and comparing nucleic acid sequences and structures formed by nucleic acid sequences.

### BACKGROUND

Nucleic acids are biopolymers made up of nucleotide monomers. Nucleotides comprise a 5-carbon sugar, a nitrogenous base, and a phosphate group. If the sugar is a ribose, the polymer is ribonucleic acid (RNA), if the sugar is deoxyribose, the polymer is deoxyribose nucleic acid (DNA). DNA contains the “genetic instructions” used in the development and function of all known living organisms. DNA can be present in a single or double strand (double helix) of nucleotides (A, C, G, T) that can form various secondary structures. According to the so-called central dogma of molecular biology, DNA is transcribed into RNA, and RNA is translated to amino acids. RNA is a polymeric molecule that is also essential in various biological roles. RNA consists of a single strand of nucleotides (A, C, G, U) that can fold and bond to itself through base pairing. Initially, RNA was regarded as a simple messenger—the conveyor of genetic information from its repository in DNA to the ribosomes. Over the last several decades, however, researchers have discovered an increasing number of important roles for RNA. RNAs have been found to have a variety of roles and functions, including catalytic activities, participating in processing of messenger RNAs (mRNA), helping maintain the telomeres of eukaryotic chromosomes, and influencing gene expression in multiple ways (Darnell, 2011). The specific shape into which RNA folds is known to play a major role in the molecule's function, which makes the fields of RNA folding and structure of prime interest to scientists. A more detailed and complete understanding of RNA's three-dimensional structure will allow a greater understanding of RNA function. However, obtaining such three-dimensional structure through crystallization studies is costly and time consuming, and often gives rise to coarse and grainy secondary structures. Generally, the secondary structure is defined as a set of base pairs, with additional noncrossing constraint, i.e., assuming that there are no crossing base pairs in the structure.

Various methods have been developed in an attempt to make RNA secondary structure prediction more feasible. Currently, thermodynamic-based RNA folding schemes are the commonly used methods for secondary structure prediction. Waterman (Waterman, 1978) developed the dynamic programming (DP) method that predicts a structure that minimizes the free energy of the molecule among all compatible structures. Fairly accurate energy values are available for computing loop-based minimum free energy (mfe or MFE) (Mathews et al., 1999, 2004; Turner and Mathews, 2010; Parisien and Major, 2008), which are employed by known folding methodologies (Zucker and Stiegler, 1981; Hofacker et al., 1994).

A deterministic folding methodology such as mfe-folding naturally induces a genotype to phenotype (sequence to structure) map, in which the pre-image of a structure is called a neutral network. The idea of a neutral network is closely related to the neutral theory of Motoo Kimura (Kimura, 1968), which stipulates that almost all of genotypic evolution is achieved by neutral evolution, namely, evolution without changing phenotype. Neutral networks have been studied theoretically via random graph theory (Reidys, 1997, 2002, 2009), computationally (Reidys et al., 1997; Grüner et al., 1996a, b) and by exhaustive enumeration (Göbel, 2000). Properties of neutral networks, like size, density, and connectivity have important biological implications. For example, a large neutral network is more evolutionary accessible than a small neutral network. Furthermore, neutral evolution occurs naturally on a connected and dense neutral network, for example, a population of RNA sequences can explore sequence space while maintaining its phenotype.

Some RNA sequences, for example, riboswitches (Sergano and Patel, 2007), exhibit multiple, distinctively different, stable configurations. In such cases, instead of looking merely at the mfe-structures, exploring the entire RNA energy landscape: the free energies of all structures with respect to the sequence, equipped with some notion of closeness on the structure space, may be desired. The idea of energy landscape has been studied to some degree in physics, chemistry, and biochemistry, and is thought to play a key role in understanding the dynamics of both RNA and protein folding (Dill et al., 1997; Onuchic et al., 1997; Martinez, 1984; Wolfinger et al., 2004). McCaskill (McCaskill, 1990) observed that the dynamic programming (DP) routine that computes the mfe-structure allows one to compute the partition function of structures for a given sequence. This allows one to explore statistical features (base pairing (BP) probability for example) of RNA energy landscape through Boltzmann sampling (Reidys and Stadler, 2001) and may lead to enhancement of structure prediction (Ding and Lawrence, 2003; Bernhart et al., 2006; Rogers and Heitsch, 2014). Other than global features, local features are of interest due to practical reasons. For example, local minimums in the energy landscape can be considered as an “energy trap,” and are crucial to understanding folding dynamics because they correspond to metastable configurations. In addition, one might be interested in statistical features on a constrained energy landscape, which corresponds to conditional distribution of these features. Statistical features, for example, mean (average) is important in understanding the distribution. For instance, the average height of the population in the US. However, a lot of information is lost by taking the average on the whole distribution. One might be interested in the average height of males in the US. In that case, using the constraint ‘male,’ that correspond to the conditional (‘male’) distribution, would lead to more detailed information. As a result, Boltzmann sampling methods that incorporate various meaningful constraints have been developed (Hofacker, et al., 1994).

Recently, the duality of the RNA energy landscape, namely, the free energies of all sequences with respect to a given structure, has been investigated. Busch and Backofen (Busch and Backofen, 2006) reported that using the mfe-sequence with respect to a given structure as a starting point significantly accelerated the inverse fold process. In other words, it appears that global minimum of the dual RNA energy landscape is often very close to the corresponding neutral network. Furthermore, Reidys, et al., (Barrett, et al., 2017) and Clote, et al., (Garcia-Martin et al., 2016), independently reported that sequences obtained from Boltzmann sampling over the dual RNA energy landscape have a high chance to fold into the corresponding structure.

Genetic robustness and plasticity can be characterized in terms of the variation of phenotype distribution induced by genotypic change (Gu et al., 2003; de Visser et al., 2003, Schlichting et al., 1998). Robustness concerns the insensitivity of a phenotype against genetic changes, while plasticity concerns the evolutionary adaptability through genetic changes. In particular, these two concepts have been explored in the context of noncoding RNA (ncRNA) (Borenstein and Ruppin, 2006; Rodrigo and Fares, 2012).

ncRNAs are known to function in aptamer binding as riboswitches, in chemical catalysis as ribozymes, and in RNA splicing such as spliceosome (Darnell, 2011; Breaker, 1996; Serganov and Patel, 2007; Breaker and Joyce, 1994). The folded structures underlay all these mechanisms, and allow the interaction with and subsequent modification of other biological molecules. The self-folding of RNA makes it an ideal object to study genotype-phenotype relations. Furthermore, the energy based prediction of RNA secondary structure, a coarse grain of the real three-dimensional structure, makes such study more feasible (Waterman, 1978; Mathews et al., 1999; Zuker and Stiegler, 1981; Hofacker et al., 1994).

### SUMMARY

In certain embodiments, the present disclosure provides a method of characterizing a nucleic acid sequence comprising determining robustness of the nucleic acid sequence based on an inverse fold rate of a sequence structure pair at a predetermined hamming distance associated with the nucleic acid sequence and determining plasticity of the genetic sequence based on base pairing (BP) probability.

In some embodiments, the present disclosure also provides a method of profiling a population of nucleic acid sequences comprising determining a hamming distance between a plurality of a subset of nucleic acid sequences of the population of nucleic acid sequences, determining an energy of structures formed by base pairs of the plurality of the subset of nucleic acid sequences, and profiling the plurality of the subset of nucleic sequences based on the determined hamming distance and the determined energy of the structure for each of the plurality of the subset of nucleic acid sequences. In some embodiments, the determination of the energy of the structure is based on specific nucleic acids that form the structure.

In certain embodiments, the present disclosure further provides a method of identifying a set of nucleic acid sequences comprising defining a predetermined hamming distance, generating a plurality of nucleic acid sequences, profiling the plurality of nucleic acid sequences to identify desired characteristics of the nucleic acid sequences, and computing a partition function based on energy of each of the plurality of nucleic acid sequences. In some embodiments, the set of nucleic acid sequences is identified from the generated plurality of nucleic acid sequences as satisfying certain criteria and/or a threshold probability based on the partition function. In some embodiments, the criteria is based on the computed energy of the nucleic acid sequence and the predetermined hamming distance. In some embodiments, the computing of the partition function comprises summing contributions of individual base pairs of the nucleic acid sequence. In certain embodiments, the computing of the partition function comprises determining a loop type formed by each base pair and successively decomposing the nucleic acid sequence and summing contributions of the loops formed by the base pairs of the nucleic acid sequence. In certain embodiments, the generated plurality of nucleic acid sequences has a Boltzman probability. In still further embodiments, the sampling of the set considers if a first nucleotide is paired with a second nucleotide to form an arc, if the first nucleotide is paired with a third nucleotide between the first and second nucleotides, or if the first nucleotide is unpaired.

In some embodiments, the present disclosure also provides a method of identifying a set of nucleic acid sequences that form desired compatible structures comprising generating a first plurality nucleic acid sequences and sampling a second plurality of nucleic acid sequences from the first plurality of nucleic acid sequences using a partition function. In some embodiments, the identified set of nucleic acid sequences form the desired compatible structures from the second plurality of nucleic acid sequences. In some embodiments, the desired compatible structures are functionally equivalent to a specific structure associated with a specific biologic function. In certain embodiments, the generated first plurality of nucleic acid sequences has a predefined energy threshold. In certain embodiments, the identified set includes nucleic acid sequences with progressively differing nucleotide sequences forming the desired compatible structures. In some embodiments, the desired compatible structures are formed by the identified set at a predefined energy threshold. In some embodiments, the desired compatible structures formed by the identified set of the nucleic acid sequences are not identical to each other. In some embodiments, energy states of the desired compatible structures formed by the plurality of nucleic acid structures in the identified set differ. In certain embodiments, the identified set progressively increases in distance from a desired structure, the distance being based on base-pair H-distances. In certain embodiments, the specific structure has a specific nucleotide sequence, and a first nucleic acid sequence in the identified set has a nucleotide sequence more distinguishable from the specific nucleotide sequence than a second nucleic acid sequence in the identified set having a lesser distance from the desired structure. In some embodiments, at least some of the desired compatible structures are identical. In some embodiments, the predefined energy threshold is a minimum free energy threshold. In some embodiments, the specific structure is formed by the generated initial population of nucleic acid sequences that are identical.

In certain embodiments, the present disclosure also provides a method of identifying a set of nucleic acid sequences that form a specific structure comprising generating an initial population of nucleic acid sequences that form the specific structure, using each of the nucleic acid sequences in the initial population of nucleic acid sequences to generate a first plurality of nucleic acid sequences from each of the nucleic acid sequences in the initial population, sampling, with a partition function, a second plurality of nucleic acid sequences from the first plurality of nucleic acid sequences that were generated from the initial population of nucleic acid sequences. In some embodiments, the identified set of nucleic acid sequences form the specific structure from the second plurality of nucleic acid sequences sampled from the first plurality of nucleic acid sequences that were generated from the initial population of nucleic acid sequences, and the specific structure formed by the identified set of nucleic acid sequences is functionally equivalent to a desired specific structure associated with a specific biologic function. In some embodiments, the specific structure is formed by the generated initial population of nucleic acid sequences formed at a predefined energy threshold. In some embodiments, the generated first plurality of nucleic acid sequences has a predefined energy threshold. In some embodiments, the identified set includes nucleic acid sequences with progressively differing nucleotide sequences forming the specific structure. In certain embodiments, the specific structures formed by the identified set of the nucleic acid sequences are not identical to each other. In certain embodiments, energy states of the specific structures formed by the plurality of nucleic acid structures in the identified set differ. In some embodiments, the identified set progressively increases in distance from the specific structure, the distance being based on base-pair H-distances. In some embodiments, the specific structure has a specific nucleotide sequence, and a first nucleic acid sequence in the identified set has a nucleotide sequence more distinguishable from the specific nucleotide sequence than a second nucleic acid sequence in the identified set having a lesser distance from the desired structure. In some embodiments, the predefined energy threshold is a minimum free energy threshold.

In some embodiments, the present disclosure also provides a method of determining similarity of two or more nucleic acid sequences comprising identifying the one or more secondary structures formed by an initial nucleic acid sequence, generating a plurality of nucleic acid sequences that form the identified one or more secondary structures formed by the initial nucleic acid sequence, and identifying at least one nucleic acid sequence from the plurality of nucleic acid sequences. In some embodiments, the similarity of the initial nucleic acid and the at least one nucleic acid sequence is based on the identified one or more secondary structures. In some embodiments, the identified at least one nucleic acid sequence has a predetermined energy threshold. In some embodiments, the identified at least one nucleic acid sequence includes two or more nucleic acid sequences, the two or more nucleic acid sequences have progressively differing nucleotide sequences forming the desired compatible structures. In some embodiments, the identified one or more secondary structures are formed by the identified one more nucleic acid sequence at a predefined energy threshold. In some embodiments, the predefined energy threshold is a minimum free energy threshold.

In certain embodiments, the present disclosure further provides a method of determining similarity of one or more secondary structures of nucleic acid sequences comprising identifying at least one initial secondary structure, generating a first plurality of nucleic acid sequences that form the identified at least one secondary structure at a first predetermined energy threshold, sampling, with a partition function, a second plurality of nucleic acid sequences from the first plurality of nucleic acid sequences, and determining a set of secondary structures formed by the second plurality of nucleic acid sequences at a second predetermined energy threshold. In some embodiments, the similarity of the at least one initial secondary structure and the set of secondary structures is based on at least one of the first and second plurality of nucleic acid sequences. In some embodiments, the first predetermined energy threshold and the second predetermined energy threshold are the same. In some embodiments, at least one of the first and second predetermined energy thresholds are a minimum free energy threshold.

### BRIEF DESCRIPTION OF THE DRAWINGS

FIG. 1A is a schematic of the secondary structure of a tRNA.

FIG. 1B is a diagrammatic representation of the secondary structure of the tRNA of FIG. 1A.

FIG. 2 is shows the IFR of the stem-loop structure corresponding to MI0005062, from 1≤k≤20, where the upper curve represents the IFR of the natural sequence structure pair. The lower curve represents the average IFR of 5 random sequences that fold into the same structure. The x-axis denotes hamming distance and the y-axis denotes IFR (%).

FIG. 3 shows the IFR (%) of sampled sequences from the natural structure of the microRNA let-7 family. The x axis is hamming distance and the y axis is IFR (%).

FIG. 4 demonstrates the distribution of base pair distance between the MFE structure of sample sequence and the reference structure of MI0000416. The x-axis denotes the relative base pair (BP) distance, the y-axis denotes the hamming distance, and the z-axis denotes the inverse fold frequency (out of 50,000).

FIG. 5 demonstrates the distribution of base pair distance between the MFE structure of sample sequence and the reference structure of MI0005057. The x-axis denotes the relative base pair distance, the y-axis denotes the hamming distance, the z-axis denotes the inverse fold frequency (out of 50,000).

FIGS. 6A, 6B, and 6C show base pairing probability of MI0005057 at different hamming distance. The upper right part of the matrices shows the base pair (BP) probability, while the lower left half shows the original structure. The scale on the right shows BP probability (from 0.0 to 1.0). (a) FIG. 6A: k=1 (b) FIG. 6B: k=5. (c) FIG. 6C: k=20.

FIG. 7 demonstrates the base pairing frequency matrix of MI0000416 at h=20. The upper right half corresponds to the base paring frequency, while the lower left part corresponds to the reference structure. The scale on the right shows BP probability (from 0.0 to 1.0).

FIG. 8 shows the IFR of MI0015952 from 120. The x-axis denotes hamming distance, and the y-axis denotes inverse fold frequency (out of 50,000)

FIG. 9 is a depiction of hairpin-, helix-, bulge-, interior- and multi-loops in secondary structures.

FIG. 10 depicts the sequence distribution of sampled sequences by Boltzmann sampler without distance filtration respect to their hamming distance to the natural one. The distance was normalized by the sequence length. The data corresponds to microRNA let-7 family (human) hsa0000064, hsa0000068, and hsa0000061. The x-axis denotes relative hamming distance, and the y-axis denotes probability density.

FIG. 11 shows that neighborhood IFR varies from different sequence structure pairs. The IFR as a function of distance of three sequence structure pairs from human microRNA let-7 family: hsa0000060 (80 nts, ●), hsa0000067 (87 nts, ▪) and hsa0000100 (119 nts, ♦) are shown. The sample size was 50,000. The x-axis denotes hamming distance, and the y-axis denotes IFR.

FIG. 12 depicts IFR (mean) of sampled sequences from the natural structure of microRNA let-7 family of three classes: human, lizard and drosophila. The length of sequences ranges from 80 nts to 120 nts. Sample size was 50,000. The x-axis denotes hamming distance, and the y-axis denotes inverse fold frequency.

FIG. 13 shows the IFR of natural sequence structure pairs versus its neighbor sequences. Natural sequence structure pair of hsa0000063 and sample 100 sequences with distance filtration 5, 7, 10 and 20 respectively were used. The x-axis denotes sorted sequence, and the y-axis denotes IFR.

FIGS. 14A and 14B depict the distribution of sequences with respect to sampling distance filtration and base pair distance between the folded structure and the natural structure. The x-axis denotes the relative base pair distance (normalized by the sequence length), the y-axis denotes the hamming distance, and the z-axis denotes the frequency of sequence.

FIGS. 15A, 15B, and 15C show the base pairing frequency matrix of MI0005057 with distance filtration 1 (FIG. 15A), 5 (FIG. 15B) and 20 (FIG. 15C). The upper right half corresponds to the base paring frequency, while the lower left part corresponds to the reference structure. The scale on the right shows BP probability.

FIGS. 16A and 16 B depict the base pairing frequency matrix of MI0000416 (left) and MI0005057 (right) with distance filtration 20. The upper right half corresponds to the base paring frequency, while the lower left part corresponds to the reference structure. The scale on the right shows BP probability.

FIG. 17 is a flowchart showing an embodiment finding a neutral path between a pair of sequences.

FIG. 18 shows a neutral path (SEQ ID NO: 1-15) found by a method described herein from the natural sequence of hsa0000063 (SEQ ID NO: 1) to a sequence having hamming distance 20 that fold to the structure of hsa0000063. The path has 14 steps (SEQ ID NO: 2-15), 8 point mutants, and 6 base pair mutants.

FIG. 19 depicts mapping sequences into structures.

FIGS. 20A and 20B show the degeneracy of natural sequences: two MFE-structures of the precursor miRNA Bos Taurus miR-2405, having base-pair distance 48.

FIG. 21 depicts iNeutrality: A and B are the MFE sets of the degenerate sequences a and σ′, the diamonds represent the MFE structures chosen by the Vienna RNA package as MFEs.

FIG. 22 shows a drift walk: σ₀ and σ₃ are non-degenerate while σ₁ an σ₂ are degenerate.

FIG. 23 depicts the frequency distribution of the sizes of the MFE sets: for random sequences of length 100 and precursor miRNAs of length at most 100.

FIG. 24 displays the distribution of base-pair distance of MFE pairs obtained from a sample of 10⁶ random degenerate RNA secondary structures.

FIG. 25 shows the secondary structure moves of type I and II: type I moves add or delete a base pair, while type II moves shift a base pair on either side without sliding over base pairs.

FIG. 26 depicts the frequency distribution of Hamming distances to the starting sequence along drift walks, random walks and neutral walks.

FIG. 27 shows the frequency distribution of Hamming distances along drift walks filtered by the base-pair H-distance.

FIG. 28 depicts the distribution of base-pair H-distances to the MFE set of the starting sequence along free drift walks of length 2000.

FIG. 29 shows the average base-pair H-distance to the MFE set of the starting sequence, σ, over Hamming distances assumed by a drift walk, random walk and monotone drift walk of 30000 steps.

FIGS. 30A and 30B depicts drift walks in the interior of neutral networks: the frequency distribution of the times (number of consecutive steps) spent in the interior of neutral networks. The insert (FIG. 30B) shows the frequency of numbers of K-switches contained in between J- and I-switches.

FIG. 31 shows the base-pair H-distance at I-switches: suppose 0<d(b,c) is less than d(a,b) and d(a,c). Then the base-pair H-distance between the MFE sets A and B equals d(b,c). Suppose at step i the MFE set changes to either {a} or {c}, i.e., we have a Type I transition. In certain embodiments, the base-pair H-distance between A and {a} is zero and the distance between A and {c} is d(a,c). Since 0<d(b,c), the H-distance decreases in case of A→{a} and since d(b,c)<d(a,c) increases in case of A→{c}.

FIG. 32 depicts the evolution of the base-pair H-distance along a particular, free drift walk having 2000 steps.

### DETAILED DESCRIPTION

### Methods and Systems for Analyzing Nucleic Acid Secondary Structures

The inference of information from genetic material (DNA or RNA) presumes the clear interpretation of what we consider as “information.” It is this particular interpretation that has shaped the current method of trying to understand genetic materials in the context of comparative genomics.

Current methods for comparing nucleic acid sequences are primarily focused on the sequence of nucleotides as source of genetic information. BLAST is one such method. BLAST is an approximation of the Waterman-Smith, polynomial time sequence-alignment method, whose underlying idea is that by inserting “gaps,” two genomic sequences are arranged, such that aligned subsequences coincide in as many nucleotides as possible. BLAST assumes that only the information contained in the actual sequence of nucleotides in the genetic material is important.

Sequence alignment can also be interpreted from an evolutionary point of view. If two sequences in an alignment share a common ancestor, mismatches can be interpreted as point mutations and gaps as indels (that is, insertion or deletion mutations in the genome) introduced in one or both lineages, in the time since they diverged from one another. Aligning two sequences can be viewed as identifying a minimal score evolutionary path problem. One of the important features of alignment is that the cost of the event is independent of the position of the nucleotide, and thus independent of any structural information.

It has been observed in biology that genomic sites evolve at vastly different rates, i.e., there is significant inhomogeneity of mutation rates of sites in genetic material. This finding raises the question as to what causes this site specific variability. To understand this, we revisit the “central dogma” of biology stipulating that coding DNA is transcribed into RNA and that RNA is then translated into proteins (structures). In other words, it is the protein structure, that is the level at which biological meaning is conveyed. The same holds, in principle, for noncoding DNA sequences; however, these do not translate into protein, but can produce RNA structures having specific biological functionalities (e.g., RNA telomerases in aging processes). In either case, it is the structure and not the sequence that carries the semantics. Current sequence analytical methods (e.g., BLAST) operate strictly on sequence level and do not incorporate protein or RNA structure. Thus, there is a need for methods and systems that incorporate structure into nucleic acid and amino acid sequence analysis. The present disclosure describes such systems and methods.

In certain embodiments, the methods and systems described herein are directed to sequence similarity measures for scoring two given nucleic acid (e.g., DNA and/or RNA) sequences. The methods and systems described herein are distinct and superior over known methods at least because the methods and systems described herein 1) incorporate sequence and phenotypic information; 2) allow inclusion of context, i.e., specific information on either of the sequences, like evolutionary context, or a priori knowledge of structural features; 3) can match sequences that are virtually alignable, vastly enhancing the bandwidth of analysis of genetic material; allow a systemic analysis of noncoding sequences, for which previous methods (e.g., BLAST) were known not to address; and suggest by construction a “dual,” i.e., immediately have an additional similarity measure of structures, allowing one to understand the way phenotypes evolve.

In embodiments, nucleic acid sequence and structure duality refer to one nucleic acid sequence has many structures and one structure has many sequences.

The methods and systems described herein can be applied, in certain embodiments, to any type of nucleic acid polymer, including but not limited to DNA, RNA, polymers containing synthetic nucleotides, and combinations thereof. In certain embodiments, the methods and systems described herein are directed to analyzing RNA nucleic acid molecules. In practice, the entire dual RNA energy landscape might be too big to study, as most of the sequences on the landscape are not evolutionary. It is often of interest to study the neighborhood of a real RNA sequence (Borenstein and Ruppin, 2006). The present disclosure described herein enables a more detailed and biologically meaningful study on the dual RNA energy landscape. Embodiments described herein are directed to an efficient Boltzmann sampling method that incorporates hamming distance constraints on the nucleotide sequence. In embodiments, only nucleotide sequences that are at (or within) an arbitrarily provided hamming distance to a reference sequence are sampled. The hamming distance between two sequences of equal length is the number of positions at which the corresponding nucleotides are different.

One can consider the sampling methods as a ‘microscope’ to study the dual RNA energy landscape. The methods and systems described herein enable the study of one or more local regions of the landscape with much higher resolution than previously unrestricted samplers or methods. Furthermore, by increasing the hamming distance in certain embodiments, the method and systems may allow one to gradually ‘zoom out’ and study a much bigger neighborhood of a sequence that cannot be achieved by other methods, such as exhaustive enumeration.

In addition, one can consider the RNA product energy landscape, the free energy of all sequence structure pairs, equipped with suitable topology (product topology for example). As discussed in (Barrett, et al., 2017), the information lies within the sequence structure pair, instead of merely the sequence or the structure. The RNA (dual) energy landscape is simply the constrained landscape of the product landscape by fixing the sequence (structure). The partition function of the entire RNA product energy landscape can be computed by the method described in (Waldispühl et al., 2008). In certain embodiments, the method and systems described herein also provide the use of a sampling method over the product landscape that incorporates both hamming distance (on the sequence) and base pair distance (on the structure) filtration.

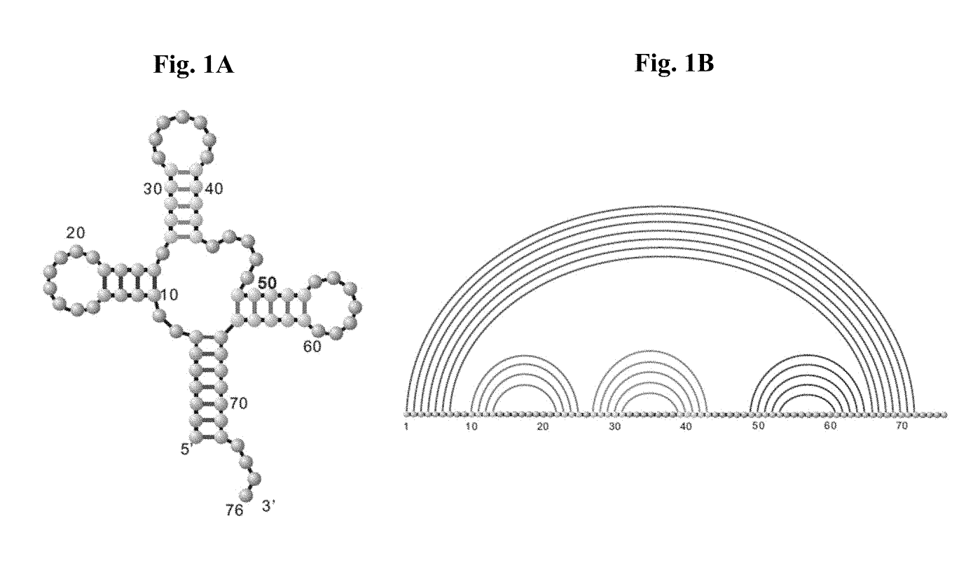

A graph presentation of RNA secondary structures is provided in FIGS. 1A and 1B. RNA secondary structures are represented by a diagram, where vertices are drawn in a horizontal line and arcs in the upper half-plane. In the diagram, vertices represent nucleotides and arcs represent base pairs. Vertices are labeled by [n]={1, 2, . . . , n} from left to right, indicating the orientation of the nucleotide strand backbone from 5′-end to 3′-end. A base pair, denoted by (i, j) is an arc connecting vertices labeled by i and j. Two arcs (i, j) and (r, s) are deemed to be crossing if for i<r, i<r<j<s holds. An RNA secondary may be referred to as pseudoknot-free if no crossing arcs are contained within the structure. Furthermore, arcs in a secondary structure induce the partial order: (r, s) [symbol] (i, j) if i<r<s<j holds.

A partition function describes the statistical properties of a system at thermodynamic equilibrium. The partition function facilitates the calculation of Boltzmann distributions and Boltzmann sampling. In certain embodiments, the partition function may be expressed with hamming distance filtration h on sequences and base pair distance filtration b on structures. For example, given a structure S and a reference sequence σ̅, the partition function of sequence structure pair (τ, T) with hamming distance filtration h on sequences and base pair distance filtration b on structures is expressed as:

$${Q}_{h , b} ( τ , T ) = {\sum }_{σ , {d}_{h} ( σ , τ ) = h}^{ } {\sum }_{S , {d}_{bp} ( S , T ) = b}^{ } {e}^{\frac{- η ( σ , S )}{RT}} , \qquad Equation 1$$

where η(σ, S) is the energy of S on σ, R is the universal gas constant and T is the temperature.

In some embodiments, before introducing the recursion of Q_{h,b}(τ, T), we first consider a simplified energy model with a trivial energy function η(σ, S)=1, namely, any (σ, S) pair has the same contribution. This is tantamount to enumerating the number of (σ, S) pairs that dₕ(σ,τ)=h and d_{bp}(S,T)=b. To avoid any potential notation confusion, in some embodiments, this number is denoted as N_{h,b}(τ, T).

In certain embodiments, the notation used in describing the recursion may be first clarified. Let N={A, C, G, U} and B={AU, UA, CG, GC, UG, GU} to denote the set of nucleotides and base pairs, respectively. For two aligned sequences σ₁ and σ₂, the hamming distance, denoted by dₕ(σ₁, σ₂), is the total number of positions of σ₁ and σ₂ that have different nucleotides. For a given structure S, let S denote the set of base pairs in S. For an interval [i, j] let

S_{[i,j]}={(k,l)|i≤k<l≤j,(k,l)∈S}

be the collection of base pairs whose end points are in [i, j]. The base pair distance of S₁ and S₂ is the total number of different base pairs, i.e.,

d_{bp}(S₁,S₂)=|S₁∪S₂|−|S₁∩S₂|.

In certain embodiments, the decomposition of a secondary structure can be described using a context-free grammar. In the grammar the terminal symbols {(.)} denote the left end of a base pair, an unpaired base, and the right end of a base pair, respectively. The non-terminal symbols {S, C} present an arbitrary secondary structure and one covered by a maximum arc. The production rules are S→CS|.S and C→(S). For an interval [i, j], let N_{h,b}^{S} (i,j) denote the number of (σ_{i,j}, S_{[i,j]}) pairs such that dₕ(σ_{i,j}, σ_{i,j})=h and d_{bp}(S_{[i,j]}, T_{[i,j]})=b, and N_{h,b}^{C}(i,j) denotes the (σ_{i,j}, C_{[i,j]}) pair where (i, j) is a base pair in C_{[i,j]} with the same constrains.

If i is unpaired in S_{[i,j]}, we follow the production rule S→.S. S_{[i,j]} is decomposed to an unpaired base i and S_{[i+1,j]}. Then S_{[i,j]} differs from T_{[i,j]} βᵢ=0 base pair when i is also unpaired in T_{[i,j]}, otherwise βᵢ=1. Going through all σᵢ∈N and δᵢ=1 if σᵢ≠τᵢ, otherwise δᵢ=0, we have

$${N}_{h , b} ( i , j ) = {\sum }_{{σ}_{i} ∈ N} {N}_{h}^{S} - {δ}_{i} , b - {β}_{i} ( i + 1 , j ) .$$

If i is unpaired with some k, i<k≤j, we follow the production rule S→CS. S_{[i,j]} is decomposed to a substructure C_{[i,k]}, and S_{[k+1,j]}. Suppose h=dₕ(τ_{i,j}, σ_{i,j}), h₁=dₕ(τ_{i,k}, σ_{i,k}) and h₂=dₕ(τ_{k+1,j}, σ_{k+1,j}), we have h=h₁+h₂. Similarly, if b=d_{bp}(T_{[i,j]}, S_{[i,j]}), b₁=d_{bp}(T_{[i,k]}, C_{[i,k]}) and b₂=d_{bp}(T_{[k+1,j]}, S_{[k+1,j]}), we have b=b₁+b₂+β where β is the number of conflicting base pairs in T_{[i,j]} when introducing (i, k). Hence

$${N}_{h , b}^{S} ( i , j ) = {{\sum }_{{i < k \leq j}_{{h}_{1} + {h}_{2} = h}}^{ }}_{{b}_{1} + {b}_{2} = b - β} {N}_{{h}_{1} , {b}_{1}}^{C} ( i , k ) {N}_{{h}_{2} , {b}_{2}}^{S} ( k + 1 , j ) .$$

Following the production rule C→(S), we remove (i, j) from C_{[i,j]} and it remains S_{[j+1,j-1]}. For h=dₕ(τ_{i,j}, σ_{i,j}) and h₁=dₕ(τ_{i+1,j-1}, σ_{i+1,j-1}) we have h=h₁+δᵢ+δⱼ. Similarly, For b=d_{bp}(T_{[i,j]}, S_{[i,j]}) and b₁=d_{bp}(T_{[i+1,j-1]}, S_{[j+1,j-1]}), we have b=b₁+β_{i,j} where δ_{i,j}=0 if (i,j) is also a base pair in T_{[i,j]}, and δ_{i,j}=1 if i, j are both unpaired in T_{[i,j]}. Then we have

$${N}_{h , b}^{C} ( i , j ) = {\sum }_{{σ}_{i} , {σ}_{j} ∈ N}^{ } {N}_{h - {δ}_{i} - {δ}_{j} , b - {δ}_{i , j}}^{S} ( i + 1 , j - 1 ) .$$

We next explain how to extend the recursion, in certain embodiments, with the simplified energy model to Turner's loop based energy model (Mathews et al., 1999, 2004). A loop L in a secondary structure is a sequence of intervals ([aᵢ,bᵢ])ᵢ, 1≤i≤k, where (a₁,bₖ), (bᵢ,aᵢ₊₁), ∀1≤i≤k−1, are base pairs. Because no crossing arc is allowed, nucleotides in the interval ([aᵢ+1,bᵢ−1])ᵢ are unpaired. In particular, L is called a hairpin loop if k=1, an interior loop (or a bulge loop, a helix, depending on how many unpaired vertices in the intervals) when k=2, and a multi-loop when k≥2.

Note that the arc (a₁,bₖ) is the maximum in a loop, i.e., (bᵢ,aᵢ₊₁) [symbol] (a₁,bₖ) for all 1≤i≤k−1, hence L can be represented by (a₁,bₖ). The interaction of two loops is either empty, or base pairs. Each base pair is contained in exactly two loops: one where it is the maximum one, and one where it is not. There is a particular loop called the exterior loop that consists of all maximal arcs in a secondary structure not presented by them. For convenience, in certain embodiments we draw a formal rainbow arc, denoted by (0, n+1), on top of the secondary structure, so the exterior loop is represented by the rainbow arc. In some embodiments, there are no particular nucleotides associated with position 0 and n+1.

In Turner's model (Mathews et al., 1999, 2004), the energy of a loop, η(σ, L), is computed by its loop type (hairpin, interior loop, exterior loop or multi-loop), base pairs, as well as a finite number of nucleotides contained in L. The energy of a sequence structure pair, η(σ, S), is given by the sum of energy of individual loop, i.e.,

$$η ( σ , S ) = {\sum }_{L ∈ S} η ( σ , L ) .$$

In certain embodiments, the context-free grammar is extended to be compatible with the loop based energy model (Hofacker et al., 1994). The non-terminal symbol S presenting an arbitrary secondary structure, C presenting a secondary structure covered by a maximum arc, M presenting an arbitrary secondary structure having at least two branchings, M₁ presenting a secondary structure that has a unique maximum arc. This results in the production rules

$$S \to \cdot S \vert CS$$

$$C \to ( S ) \vert ( M )$$

$$M \to {M}_{1} M$$

$${M}_{1} \to \cdot {M}_{1} \vert C$$

Let Q_{h,b}^{X}(i,j,σᵢ,σⱼ) denote the partition function of (σ_{i,j},X_{[i,j]}) which X_{[i,j]} is presented by non-terminal symbol X, with nucleotides at position i σᵢ and at position j σⱼ, where dₕ(τ_{i,j}σ_{i,j}) and d_{bp}(T_{[i,j]},X_{[i,j]}). Here we need to specify the nucleotides σᵢ σⱼ because they are involved in the energy calculation of the loop above X_{[i,j]}.

Following is an example to derive the recursion according to the above production rules. For the C_{[i,j]} we take the rule C→(S)|(M). This production ruler removes the outmost base pair, retains a loop L presented by the removed arc as well as nested substructures. Depending on the loop type, we have

$${N}_{h , b}^{C} ( i , j ; {σ}_{i} , {σ}_{j} ) = {Q}_{h , b}^{hairpin} ( i , j ; {σ}_{i} , {σ}_{j} ) + {Q}_{h , b}^{interior} ( i , j ; {σ}_{i} , {σ}_{j} ) + {Q}_{h , b}^{multiloop} ( i , j ; {σ}_{i} , {σ}_{j} ) .$$

If L is a hairpin loop, we consider all σ_{i+1,j-1}∈ [symbol] ^{j-i-1} and where dₕ(Σ_{i+1,j-1}, σ_{i+1,j-1})=h and d_{bP} (T_{[i,j]}, S_{[i,j]})=b. Therefore,

$${Q}_{h , b}^{hairpin} ( i , j ; {σ}_{i} , {σ}_{j} ) = {\sum }_{{{σ}_{i + 1 , j - 1} ∈ {j - i - 1}^{}}_{{{d}_{h} ( {𝒯}_{i} + 1 , j - 1 , {σ}_{i + 1 , j - 1} ) = h}_{{d}_{bp} ( {𝒯}_{[ i , j ]} , {𝒮}_{[ i , j ]} ) = b}}}^{ } {e}^{- \frac{η ( {σ}_{i , j} , L )}{RT}} .$$

If L is an interior loop, let (p, q) denote the maximum base pair that (p, q) [symbol] (i, j). The interior loop L contains two intervals: [i+1, p−1] and [q₁,j−1]. If p−1<i+1 or j−1<q−1 the interval is empty.

Let * be the criteria dₕ(τ_{i+1,p-1}, σ_{i+1,p-1})+dₕ(τ_{q+1,j-1}, σ_{q+1,j-1})=h−h₁−δₚ−δ_{q} and d_{bp}(T_{[i+1,p-1]}, S_{[i+1,p-1]})+d_{bp}(T_{[q+1,j-1]}, S_{[q+1,j-1]})=b−b₁. As a result, we have the recursion

$${Q}_{h , b}^{interior} ( i , j ; {σ}_{i} , {σ}_{j} ) = {\sum }_{{i < p < q < j}_{{{σ}_{i + 1 , p - 1} ∈ {p - i - 2}^{}}_{{σ}_{q + 1 , j - 1} ∈ {q - j - 2}^{} , ★}}}^{ } {e}^{- \frac{η ( {σ}_{i , j} , L )}{RT}} {Q}_{{h}_{1} , {b}_{1}}^{C} ( p , q ; {σ}_{p} , {σ}_{q} ) .$$

If L is a multiloop, then

$${Q}_{h , b}^{multiloop} ( i , j ; {σ}_{i} , {σ}_{j} ) = {\sum }_{{σ}_{i + 1} , {σ}_{j - 1} ∈}^{ } {e}^{- \frac{η ( {σ}_{i , j} , L )}{RT}} {Q}_{h - {δ}_{i} - {δ}_{j} , b - β}^{M} ( i + 1 , j - 1 ; {σ}_{i + 1} , {σ}_{j - 1} )$$

For S_{[i,j]} we take the rule S→.S|CS. Therefore, we have

$${Q}_{h , b}^{S} ( i , j ; {σ}_{i} , {σ}_{j} ) = {\sum }_{{σ}_{i + 1} ∈}^{ } {Q}_{h - {δ}_{i} , b - {β}_{i}}^{S} ( i + 1 , j ; {σ}_{i + 1} , {σ}_{j} ) + {\sum }_{{{σ}_{k} , {σ}_{k + 1} ∈}_{{{h}_{1} + {h}_{2} = h}_{{{b}_{1} + {b}_{2} = b - β}_{i < k \leq j}}}}^{ } {Q}_{{h}_{1} , {b}_{1}}^{C} ( i , k ; {σ}_{i} , {σ}_{k} ) {Q}_{{h}_{2} , {b}_{2}}^{S} ( k + 1 , j ; {σ}_{k + 1} , {σ}_{j} ) .$$

Similarly, we have

$${Q}_{h , b}^{M} ( i , j ; {σ}_{i} , {σ}_{j} ) = {\sum }_{{{σ}_{k} , {σ}_{k + 1} ∈}_{{{h}_{1} + {h}_{2} = h}_{{{b}_{1} + {b}_{2} = b - β}_{i < k < j}}}}^{ } {Q}_{{h}_{1} , {b}_{1}}^{{M}_{1}} ( i , k ; {σ}_{i} , {σ}_{k} ) {Q}_{{h}_{2} , {b}_{2}}^{M} ( k + 1 , j ; {σ}_{k + 1} , {σ}_{j} ) , \\ and$$

$${Q}_{h , b}^{{M}_{1}} ( i , j ; {σ}_{i} , {σ}_{j} ) = {\sum }_{{σ}_{i + 1} ∈}^{ } {Q}_{h - {δ}_{i} , b - {β}_{i}}^{{M}_{1}} ( i + 1 , j ; {σ}_{i + 1} {σ}_{j} ) + {Q}_{h , b}^{C} ( i , j ; {σ}_{i} , {σ}_{j} ) .$$

Although a loop may contain an infinite number of nucleotides, in certain embodiments the calculation of η(σ, L) considers at most eight specific nucleotides as well as the number of base pairs and unpaired bases in L. Therefore, in some embodiments it takes constant time to compute η(σ, L) (Mathews et al., 1999, 2004). In certain embodiments, the recursion contains at most one loop index, hence the time complexity of computing Qₕ(S, σ̅) is O(n). The sample process is a standard Boltzmann sampling process, and, therefore, takes n log(n) time (Ponty, 2008; Nebel et al., 2011).

In certain embodiments, the sampling procedure follows the classical stochastic backtracking method introduced by Tacker et al. and Ding and Lawrence (Tacker et al., 1996; Ding and Lawrence, 2003).

### Methods and Systems for Analyzing Robustness and Plasticity of RNAs

Generally, structures are fundamental in determining the potential function of RNAs, including ncRNAs. One manner for characterizing the robustness and plasticity of an RNA is in terms of the variation of secondary structure distribution. In certain aspects, robustness, as used herein, refers to the stability of a secondary structure against genetic changes, whereas plasticity, in certain aspects, refers to diversity of the structural configurations. Because structures are often crucial to the function of ncRNAs, the robustness and plasticity of ncRNAs are likely fundamental components of the fitness of these molecules. The fitness of a molecule refers to the ability of the molecule to survive and be replicated or reproduced. In this regard, many researchers have attempted to identify the footprints of natural selection on secondary structures of small non-coding RNAs (sncRNAs) (Borenstein and Ruppin 2006; Rodrigo and Fares, 2012). In particular, Borenstein and Ruppin (Borenstein and Ruppin, 2006) define neutrality of an RNA sequence α=a₁ a₂ . . . aₙ by

$$η ( σ ) = 1 - \frac{< d >}{n} ,$$

where <d> denotes the average, taken over all 3n single-point mutants of σ, of the base pair distance d between the minimum free energy (MFE) structure S₀ of σ and the MFE structures of single-point mutants of σ. An RNA sequence σ is then defined to be robust if η(σ) is greater than the average neutrality of 1000 control sequences generated by the program RNAinverse (Lorenz et al., 2011), which fold into the same target structure S₀. The main finding of Borenstein and Ruppin (Borenstein and Ruppin, 2006) is that precursor microRNAs (pre-miRNA) exhibit a significantly higher level of mutational robustness than random RNA sequences having the same structure. Subsequently, Rodrigo and Fares (Rodrigo and Fares, 2012) undertook a similar analysis for bacterial small RNAs, but using different definitions. The main finding of Rodrigo and Fares (Rodrigo and Fares, 2012) was that bacterial sncRNAs are not significantly robust when compared with 1000 sequences having the same structure, as computed by RNAinverse. However, bacterial sncRNAs tend to be significantly plastic, in the sense that the ensemble of low energy structures is structurally diverse. Rodrigo and Fares (Rodrigo and Fares, 2012) used definitions based on the notion of ensemble diversity as earlier defined in Gruber et al. (Gruber et al., 2008).

In this work, the definitions of robustness were based on taking the average of all 3n single-point mutants. The underlining assumption is that all 3n point mutations are equally likely to occur. Moreover, their definitions only consider point mutations while not taking multiple mutations into consideration, as the number of sequences that need to be considered will grow exponentially. In the neutral theory (Kimura, 1968), Motoo Kimura stipulates that almost all genotypic evolution is achieved by neutral evolution. A necessary condition for neutral evolution is that the mutation must be compatible to the structure. Recent results show that secondary structures have genuine influence on selection in RNA genes. Hein et al. (Mimouni et al., 2008) classify stem positions into structural classes and validate that they are under different selective constraints. In the study by Price et al. (Price et al., 2011), the authors observed neutral evolution in Drosophila microRNA and the evolution increased the robustness of the RNA. Another result relates to Human Accelerated Regions (HAR) in the brains of primates. HAR are noncoding RNAs with an accelerated rate of nucleotide substitution along the lineage between humans and chimpanzees. It has been suggested that the increased rate of nucleotide substitutions is of importance for human brain evolution (Pollard et al., 2006; Benjaminov et al., 2008). Moreover, the most divergent of these regions, HAR1, has been biochemically confirmed to fold into distinct RNA secondary structures in humans and chimpanzees. It is believed that the mutations in the human HAR1 sequence, compared to the chimpanzee sequence, actually stabilize this RNA structure. These results suggest the existent of shape modulated evolution.

Embodiments described herein include a novel approach of using a Boltzmann distribution of sequences with respect to a given structure. Aspects of the disclosure include methods and systems that use a Boltzmann sampler of sequences with respect to a given structure, which demonstrates that natural structures have higher inverse fold rate (IFR) (Equation 4) than random structures. The inventors then considered the sequence-structure pair as the unit of evolution, instead of solely considering the sequence. In addition, the inventors investigated the robustness and plasticity of natural sequence-structure pair under the shape modulated evolution that is based on an established energy model.

Robustness and plasticity have been explored in the context of noncoding RNA (ncRNA) (Borenstein and Ruppin, 2006; Rodrigo and Fares, 2012). The authors investigated if single point-mutation would affect the folded structure of ncRNA. In their framework, all point-mutations are equally likely to occur hence the structure does not affect the mutation rate. In certain embodiments, the disclosure describes methods and systems that employ a novel approach to robustness and plasticity that incorporates both sequence and structure information. Revisiting some of the basic concepts in thermodynamic models of RNA secondary structure and MFE folding approach can be helpful.

An energy model rl can be considered as a sequence-structure pairing:

η:Q₄ⁿ×Sₙ→R␣+∞,

where Q₄ⁿ and Sₙ denote the space of sequences, a, and the space of secondary structures, S, respectively and η(σ, S) is the energy of S on σ. Note that in most energy model, if a sequence σ is not compatible with a structure S, then η(σ,S)=+∞.

Then, MFE folding is a map:

$$mfe \text{:} {Q}_{4}^{n} \to {S}_{{n}^{′}} \\ σ \to {arg min}_{S ∈ {𝒮}_{n}} η ( σ , S ) . \qquad Equation 2$$

Recall Borenstein and Ruppin's definition of neutrality, in which the neutrality is obtained by taking the average over all 3n point mutants. Because, in certain aspects, we are interested in mutations with respect to a fixed structure, we therefore only consider the compatible mutations in such aspects. Furthermore, to incorporate the thermal dynamic information into consideration, we introduce the notion of the Boltzmann distribution of mutations of a sequence-structure pair. First, let us look at the partition function of k-point mutations of a sequence-structure pair, namely, the partition function for all sequences that is at hamming distance k to the given sequence with respect to the given structure.

In certain embodiments, let σ be a sequence over n nucleotides and let S be its associated structure. Then the partition function of k-point mutants of σ with respect to S is given by:

$${Q}_{k} ( σ , S ) = {\sum }_{{σ}^{′} , h ( σ , {σ}^{′} ) = k} {e}^{- \frac{η ( {σ}^{′} , 𝒮 )}{RT}} , \qquad Equation 3$$

where h is the hamming distance, η(σ′, S) is the energy of S on σ′, R is the universal gas constant and T is the temperature.

This is essentially the “dual” of McCaskills partition function with hamming distance h=k filtration. Given the partition function, we can define the Boltzmann distribution of k-point mutant of σ with respect to S: the probability of a specific k-point mutant σ* of σ with respect to S is given by:

$${Pr}_{k} ( {σ}^{*} \vert σ , S ) \frac{{e}^{- \frac{η ( {σ}^{*} , S )}{RT}}}{{ }^{-} {Q}_{k} ( σ , S )} . \qquad$$

In certain embodiments, we are after the genetic robustness, and in such embodiments, we are interested in those k-point mutants that fold back to S. By summing over the probability of point mutants a that fold into S, we obtained the inverse fold rate (IFR) of the sequence structure pair (σ, S) at h=k, and we can use IFR to quantify the robustness of a sequence structure pair.

$${IFR}_{k} ( σ , S ) = {\sum }_{mfe ( {σ}^{*} ) = S} {Pr}_{k} ( {σ}^{*} \vert σ , S ) . \qquad Equation 4$$

Note that the IFR at h=1 is a modification of Borenstein and Ruppin's definition of neutrality. Instead of using a uniform probability distribution of all 3n point mutants, certain embodiments of the disclosure use a Boltzmann weighted distribution. By doing so, such embodiments automatically rule out the incompatible point mutations. Furthermore, these embodiments compensate energetically favorable mutations and penalize destabilizing mutations. Moreover, instead of using base pair distance as the metric on structure space, such embodiments use a discrete metric, because in certain embodiments the robustness of a fixed structure is of interest. In some embodiments, IFR is used to measure the robustness of a sequence structure pair.

Traditionally, plasticity has been studied in the context of Boltzmann ensembles of structures corresponding to a sequence. Different notions of plasticity have been introduced (Rodrigo and Fares, 2012; Gruber et al., 2008) to extract the structural diversity in the ensemble. In particular, base pairing probability has been used to study the Boltzmann ensemble (Hofacker et al., 2004; Bernhart et al., 2006). Certain embodiments of the disclosure are directed to ‘mutation plasticity.’ In other words, some embodiments are directed to characterizing the phenotypic ‘search radius’ of evolution. In certain embodiments, by MFE folding the Boltzmann distribution of point mutants of a with respect to S, an ensemble of structures is immediately obtained, from which, k-mutation base pairing probability can be defined:

$${Pr}_{k} ( ( i , j ) \vert σ , S ) = {\sum }_{( i , j ) ∈ mfe ( {σ}^{*} )} {Pr}_{k} ( {σ}^{*} \vert σ , S ) . \qquad Equation 5$$

In certain embodiments, this k-point mutant base pairing (BP) probability to study the plasticity of a sequence structure pair can be facilitated.

### Computing IFR and BP Probability by Boltzmann Sampling

In certain embodiments, the definition of both IFR and BP probability involve folding, hence, computing them strictly may be impractical. However, in certain embodiments both IFR and BP probability can be approximated efficiently using Boltzmann sampling. In some embodiments, a constrained version of the Boltzmann sampler described above can be implemented by introducing a new index corresponding to hamming distance. For example, for a length n structure, the partition function of k-point mutants in O(k²n) and sample a sequence in O(nlogn) can be computed. As a result, in certain embodiments, the main time complexity is in the folding part (O(n³)).

Methods and Systems Using Efficient Dual Sampling with Hamming Distance Filtration

Finding a minimum free energy (mfe) sequence for a given structure has been investigated in using dynamic programming (DP) (Busch and Backofen, 2006). That approach facilitates the arc decomposition of a secondary structure (Waterman, 1978) to compute mfe subsequence recursively. Later developments computed the partition function of a given structure following a similar routine (Garcia-Martin et al., 2016; Barrett et al., 2017). Methods that sample RNA sequences with Boltzmann distribution based on the computed partition function are given in Garcia-Martin et al., 2016 and Barrett et al., 2017. In the present disclosure, we provide systems and methods that not only sample RNA sequences from the given structure S with Boltzmann distribution, but the sampled sequences also have a fixed hamming distance d to the given reference sequence σ̅. The methods and systems described herein include introducing a new parameter h associated to a subsequence, indicating the hamming distance between the subsequence and the reference sequence.

In FIG. 1B, RNA secondary structures are represented by a diagram, where vertices are drawn in a horizontal line and arcs in the upper half-plane. In the diagram, vertices represent nucleotides and arcs represent base pairs. Vertices are labeled by [n]={1, 2, . . . , n} from left to right, indicating the orientation of the nucleotide backbone from 5′-end to 3′-end. A base pair, denoted by (i, j) is an arc connecting vertices labeled by i and j. Two arcs (i, j) and (r, s) are called crossing if for i<r, i<r<j<s holds. An RNA secondary structure is referred to as pseudoknot-free if no crossing arcs are present. Furthermore, arcs in a secondary structure induce the partial order: (r, s) [symbol] (i, j) if i<r<s<j holds.

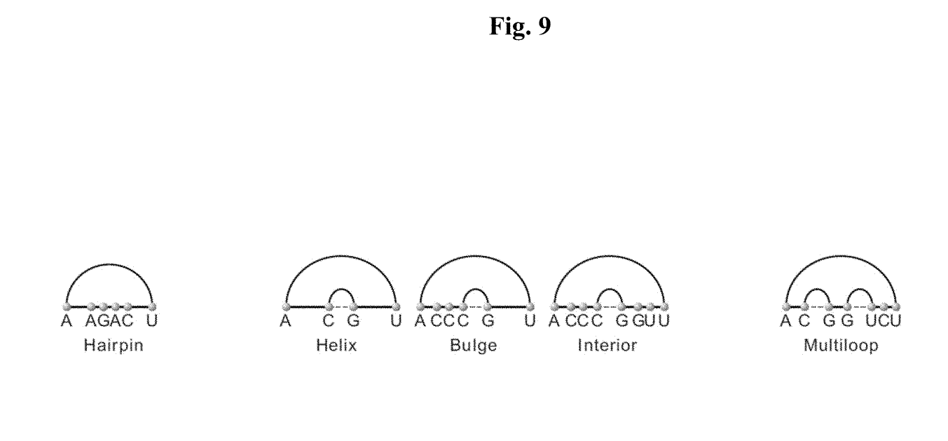

The energy of a sequence structure pair η(σ, S) is first computed by the sum of energy contribution of its individual base pairs (Nussinov et al., 1978). A more accurate model (Mathews et al., 1999; Mathews et al., 2004) considers energy from the loops formed by base pairs. A loop L in a secondary structure is a sequence of intervals ([aᵢ,bᵢ])ᵢ, 1≤i≤k, where (aᵢ,bₖ), (bᵢ,aᵢ₊₁), ∀1≤i≤k−1, are base pairs. Because no crossing arc is allowed, nucleotides in the interval ([aᵢ+1,bᵢ−1])ᵢ are unpaired. In particular, L is called a hairpin loop if k=1, an interior loop (or a bulge loop, a helix, depending on how many unpaired vertices in the intervals) when k=2, and a multi-loop when k≥2. See FIG. 9. Note that the arc (a₁, bₖ) is the maximum in a loop, i.e., (bᵢ,aᵢ₊₁) [symbol] (a₁,bₖ) for all 1≤i≤k−1, hence L can be represented by (a₁,bₖ). The interaction of two loops is either empty, or base pairs. Each base pair is contained in exactly two loops: the one where it is the maximum, and the other where it is not. There is a particular loop called an exterior loop that consists of all maximal arcs in a secondary structure but is not presented by them. For convenience, we have provided a formal rainbow arc, denoted by (0, n+1), on top of the secondary structure, so the exterior loop is represented by the rainbow arc. There are no particular nucleotides associated with position 0 and n+1.

In Turner's model (Mathews et al., 1999; Mathews et al., 2004), the energy of a loop, η(σ, L), is computed by its loop type (hairpin, interior loop, exterior loop or multi-loop), base pairs, as well as particular nucleotides. The energy of a sequence structure pair equals the sum of energy of all loops, i.e.,

$$η ( σ , S ) = {\sum }_{L ∈ S} η ( σ , L ) .$$

A secondary structure can be decomposed by removing arcs from outside to inside successively, see FIG. 9. When a base pair is removed, the loop it represents is also removed from the structure. Assume S_{i,j} is a substructure and (i, j) is a base pair. Let L be the loop (i, j). Assume (pᵣ, qᵣ). ∀1≤r≤k are base pairs in L different from (i,j). Then we have

$$η ( σ , {S}_{i , j} ) = η ( σ , L ) + {{\sum }_{s = 1}}^{k} η ( σ , {S}_{{p}_{r} , {q}_{r}} ) .$$

By applying the arc removal, a structure can be decomposed into loops where each loop is presented by a base pair in the structure.

Given a structure S and a reference sequence σ̅, the partition function of S with hamming distance filtration h to ā is

$${Q}_{h} ( S , \overline{σ} ) = {\sum }_{σ , d ( σ , \overline{σ} ) = h} {e}^{\frac{- η ( σ , S )}{RT}} , \qquad Equation 6$$

where η(σ, S) is the energy of S on σ, d(σ, σ̅) denotes the hamming distance between σ and σ̅, R is the universal gas constant and T is the temperature.

Qₕ(S, σ̅) is computed following the secondary structure decomposition recursively. Let Qₕ(Nᵢ,Nⱼ|S_{i,j}, σ̅_{i,j}) denote the partition function of S_{i,j} with hamming distance filtration h to the reference's subsequence σ̅_{i,j}, where the nucleotides in position i and j are Nᵢ and Nⱼ, Nᵢ,Nⱼ∀{A,U,C,G}.

Consider a minimum arc with respect to [symbol] . The arc presents a hairpin loop because there are no further arcs below it. Therefore, we consider the set of subsequences A(Nᵢ,Nⱼ,h)={σ_{i,j}|d(σ_{i,j}, σ̅_{i,j}=h,σᵢ=Nᵢ,σⱼ=Nⱼ} so we have the base case

$${Q}_{h} ( {N}_{i} , {N}_{j} \vert {S}_{i , j} , {\overline{σ}}_{i , j} ) = {e}^{\frac{- η ( σ , {S}_{i , j} )}{RT}} ,$$

For an arc (i, j) that is not minimum, let L be the loop (i, j) present and (pᵣ,qᵣ), ∀1≤r≤k, are the other arcs contained in L. Assume d(σₚ_{ᵣ}_{,q}_{ᵣ}, σ̅ₚ_{ᵣ}_{,q}_{ᵣ})=hᵣ and d(σ_{i,j}, σ̅_{i,j})=h, the remaining nucleotides in L excluding σₚ_{ᵣ} and σ_{q}_{ᵣ} have

$$t = h - {\sum }_{r = 1}^{k} {h}_{r}$$

differences. We denote the set of nucleotides having such property as

$$A ( {N}_{i} , {N}_{j} , t ) = \{ {σ}_{i} ∈ L \vert {σ}_{i} = {N}_{i} , {σ}_{j} = {N}_{j} , {\sum }_{i} d ( {σ}_{i} , {\overline{σ}}_{i} ) = t , i \neq {p}_{r} , {q}_{r} , ∀ 1 \leq r \leq k \} .$$

We have the recursion

$${Q}_{h} ( {N}_{i} , {N}_{j} \vert {S}_{i , j} , {\overline{σ}}_{i , j} ) = {\sum }_{{h}_{r} \leq h , {\sum }_{r} {h}_{r} \leq h} {\sum }_{ ( {N}_{i} , {N}_{j} , h - {\sum }_{r} {h}_{r} )} {e}^{\frac{- η ( {σ}_{i , j} , L )}{RT}} \cdot {\prod }_{r = 1}^{k} {Q}_{{h}_{r}} ( {N}_{{p}_{r}} , {N}_{{q}_{r}} \vert {S}_{{p}_{r} , {q}_{r}} , {\overline{σ}}_{{p}_{r} , {q}_{r}} ) .$$

We need to partition h into k+1 parts when computing the recursion. It is not efficient to go through all possible cases given that the number of partitions grows exponentially with k. Here we introduce an intermediate term Qₕ(Nᵢ,Nⱼ|S_{i,j}, σ̅_{i,j}) where (i, j) is not an arc and S_{i,j} is a substructure that contains no arc whose endpoint is outside S_{i,j}. If i is paired, assume (i, k) is an arc, l<k<j. Then we have

$${Q}_{h} ( {N}_{i} , {N}_{j} \vert {S}_{i , j} , {\overline{σ}}_{i , j} ) = {\sum }_{{N}_{k} , {N}_{k + 1} , 0 \leq t \leq h} {Q}_{t} ( {N}_{i} , {N}_{k} \vert {S}_{i , k} , {\overline{σ}}_{i , k} ) \cdot {Q}_{h - t} ( {N}_{k + 1} , {N}_{j} \vert {S}_{k + 1 , j} , {\overline{σ}}_{k + 1 , j} ) .$$

Otherwise if i is unpaired, depending on whether σᵢ and Nᵢ are the same, we have

$${Q}_{h} ( {N}_{i} , {N}_{j} \vert {S}_{i , j} , {\overline{σ}}_{i , j} ) = \{ \begin{matrix}{\sum }_{{N}_{i + 1}} {Q}_{h} ( {N}_{i + 1} , {N}_{j} \vert {S}_{i + 1 , j} , {\overline{σ}}_{i + 1 , j} ) & if {N}_{i} = {\overline{σ}}_{i} , \\ {\sum }_{{N}_{i + 1}} {Q}_{h - 1} ( {N}_{i + 1} , {N}_{j} \vert {S}_{i + 1 , j} , {\overline{σ}}_{i + 1 , j} ) & if {N}_{i} \neq {\overline{σ}}_{i} .\end{matrix}$$

Following the recursion, Qₕ(Nⱼ,Nⱼ|S, σ̅) is computed from bottom to top. The recursion stops when reaching the rainbow arc (0, n+1). The partition function of S with hamming distance filtration h to σ̅ a is given by Qₕ(S, σ̅)=Qₕ(N₀,Nₙ₊₁|S, σ̅), where N₀ and Nₙ₊₁ are formal symbols.

Having computed the partition function Qₕ(S, σ̅), we are in position to Boltzmann sample RNA sequences having a fixed hamming distance h to σ̅ from S, i.e., a sequence σ is sampled by the probability

$$( σ ) = \frac{{e}^{\frac{- η ( σ , S )}{RT}}}{{Q}_{h} ( S , \overline{σ} )} , d ( σ , \overline{σ} ) = h . \qquad Equation 7$$

The sampling process reverse the computational order of computing Qₕ(S, σ̅). First, we have a pool storing substructure S_{i,j} where σᵢ, σⱼ a are sampled. Furthermore, the subsequence σ_{i,j} to be sampled has h differences from the reference subsequence σ̅_{i,j}. The pool is initialized by putting S_{0,n+1}, where σ₀ and σₙ₊₁ are set to be formal symbols, d(σ, σ̅)=h.

Next we take a substructure S_{i,j} from the pool and sample nucleotides based on three scenarios:
If (i,j) is an arc. Suppose (i,j) presents a loop L and (pᵣ,qᵣ), for 1≤r≤k are the other arcs in L. Then σᵢ₊₁ and σⱼ₋₁ as well as a sequence of numbers (hᵣ)ᵣ are sampled by the probability

$$( {σ}_{i + 1} = {N}_{i + 1} , {σ}_{j - 1} = {N}_{j - 1} , {( {h}_{r} )}_{r} ) = \frac{{e}^{\frac{- η ( σ , L )}{RT}} \cdot {Q}_{h - t} ( {N}_{i + 1} , {N}_{j - 1} \vert {S}_{i + 1 , j - 1} , {\overline{σ}}_{i + 1 , j - 1} )}{{Q}_{h} ( {N}_{i} , {N}_{j} \vert {S}_{i , j} , {\overline{σ}}_{i , j} )} .$$

The substructures Sₚ_{ᵣ}_{q}_{ᵣ}, for 1≤r≤k are put into the pool.
If i is paired with some k where i<k<j. Then σₖ, σₖ₊₁ and a number t are sampled by the probability

$$( {σ}_{k} = {N}_{k} , {σ}_{j - 1} = {σ}_{k + 1} , t ) = \frac{{Q}_{t} ( {N}_{i} , {N}_{k} \vert {S}_{i , k} , {\overline{σ}}_{i , k} ) \cdot {Q}_{h - t} ( {N}_{k + 1} , {N}_{j} \vert {S}_{k + 1 , j} , {\overline{σ}}_{k + 1 , j} )}{{Q}_{h} ( {N}_{i} , {N}_{j} \vert {S}_{i , j} , {\overline{σ}}_{i , j} )}$$

The substructures S_{i,k} and S_{k+1,j} are put into the pool.
If i is unpaired. Then σᵢ₊₁=Nᵢ₊₁ is sampled by the probability

$$( {σ}_{n + 1} = {N}_{i + 1} , h ) = \{ \begin{matrix}\frac{{Q}_{h - 1} ( {N}_{i + 1} , {N}_{j} \vert {S}_{i + 1 , j} , {\overline{σ}}_{i + 1 , j} )}{{Q}_{h} ( {N}_{i} , {N}_{j} \vert {S}_{i , j} , {\overline{σ}}_{i , j}} , & if {N}_{i} \neq {\overline{σ}}_{i} , \\ \frac{{Q}_{h} ( {N}_{i + 1} , {N}_{j} \vert {S}_{i + 1 , j} , {\overline{σ}}_{i + 1 , j} )}{{Q}_{h} ( {N}_{i} , {N}_{j} \vert {S}_{i , j} , {\overline{σ}}_{i , j}} , & if {N}_{i} = {\overline{σ}}_{i} .\end{matrix}$$

The substructures S_{i+1,j} is put into the pool.

In certain embodiments, the sampling process iterates the three scenarios and at each step, the substructures in the pool become smaller. The process stops when the pool is empty.

We show that the sampling process samples a sequence with Boltzmann probability as we expected. Reviewing the sampling process and the three scenarios, we find that when a substructure S_{i,j} is put into the pool, the numerator of the probability contains the term Qₕ(Nᵢ,Nⱼ|S_{i,j}, σ̅_{i,j}, while it is taken out from the pool, the denominator of the probability contains the same term. When produced through all probability in the sampling process, this term appears exactly once in the denominator and numerator respectively. Hence, they are reduced and the numerator remains

$${Π}_{L ∈ S} {e}^{\frac{- η ( σ , L )}{RT}} = {e}^{\frac{- η ( σ , S )}{RT}}$$

and the denominator remains Qₕ(S, σ̅). Therefore, a sequence sampled by the process with probability

$${e}^{\frac{η ( σ , S )}{RT}} / {Q}_{h} ( S , \overline{σ} ) .$$

Though a loop may contain an infinite number of nucleotides, the calculation of η(σ, L) considers, in certain embodiments, at most eight specific nucleotides as well as the number of base pairs and unpaired bases in L. Therefore, it takes constant time to compute η(σ, L) (Mathews et al., 1999; Mathews et al., 2004). The recursion contains at most one loop index, hence the time complexity of computing Qₕ(S, σ̅) is O(n). The sample process is a Boltzmann sampling process, hence it takes n log (n) time (Ponty, 2008; Nebel et al., 2011).

Neutrality and Continuity in RNA Evolution: Drift Walks and iNeutrality

Disclosed herein is a new class of walks in sequence space. These drift walks generalize neutral walks and traverse iNeutral neighbors, where two sequences are called iNeutral if their sets of minimum free energy (MFE) structures have a non-empty intersection. A priori, RNA folding methods and systems select one particular MFE structure, thus reducing iNeutrality to neutrality. As described herein, we demonstrate that, in certain embodiments, 29.5% of random RNA sequences have more than one MFE structure. In addition, analysis of the statistical properties of these degenerate sequences is provided herein. In certain embodiments, provided herein is an analysis of drift walks by means of Hamming distance and base-pair H-distance, the latter, in certain aspects, being an induced Haussdorf metric on sets of RNA secondary structures. Provided herein are contrast drift walks with neutral walks as well as random walks and analysis as to how the phenotypes evolve along drift walks. The latter can travel between neutral networks and, as a result, an analysis of how much time spent in the interior of neutral networks (at sequences with unique MFE structures) and to what extent they change the associated MFE set, when they are in the boundary of neutral nets (at sequences with multiple MFE structures) is provided. In addition, provided herein is a description of the results and placing them into context with neutrality and continuity in evolution of RNA.

Traditionally, RNA sequences and RNA secondary structures give rise to a genotype-phenotype map, in which the set of all sequences that fold into a single MFE-structure is called a neutral network. Neutral networks have been considered to some degree theoretically via random graph theory (Reidys et al., 1997), computationally and by exhaustive enumeration (Grüner et al., 1996). A parameter here is the number of point mutants of an RNA sequence, folding into the same structure, called the neutral neighbors. Random graph analysis identified threshold values for connectivity and density of neutral networks, suggesting that sequence space can be traversed by neutral walks, i.e., walks along which all sequences fold into a fixed secondary structure (Schuster et al., 1994; Reidys, 2011).

In certain aspects, neutral networks of RNA structures can be considered closely related to the concept of the neutral theory of Motoo Kimura (Kimura, 1983), stipulating that evolution is achieved by neutral mutations. Neutral evolution occurs naturally on a connected and dense neutral network; a population of RNA sequences can explore sequence space while maintaining its phenotype.

Certain embodiments of the disclosure are directed to the basic idea of neutrality by incorporating, on some level, the multiplicity of phenotypes. McCaskill (McCaskill, 1990) observed that the basic recursions that compute the MFE-structure produced the entire partition function of structures. That is an energy-weighted probability space of compatible structures, whose elements could be generated by Boltzmann sampling (Haslinger and Stadler, 1999). Some embodiments are directed to a particular feature of the Boltzmann ensemble, namely the potential multiplicity of MFE-structures. Among computational biologists it is commonly believed that a MFE-structure of a sequence is unique (Schuster et al., 1994) and that, even if multiple MFE-structures exist, they are very similar. Notable exceptions exist however, for example, ribo-switch RNAs (these RNA structures are experimentally verified and not MFE-structures), i.e., sequences that exhibit two, distinctively different, stable configurations such as Leptomonas collosoma. LeCuyer and Crothers (LeCuyer and Crothers, 1993) demonstrated experimentally that the spliced leader RNA of Leptomonas collosoma has two different structures that differ only slightly in stability. These two structures have very close free energies according to the mfold package (Ding and Lawrence, 2003), but neither of them coincides with the a priori predicted MFE-structure. In certain aspects of the disclosure herein, we demonstrate that the multiplicity of MFE-structures is a prominent feature having remarkable consequences and that plays a central role in the context of evolutionary optimization.

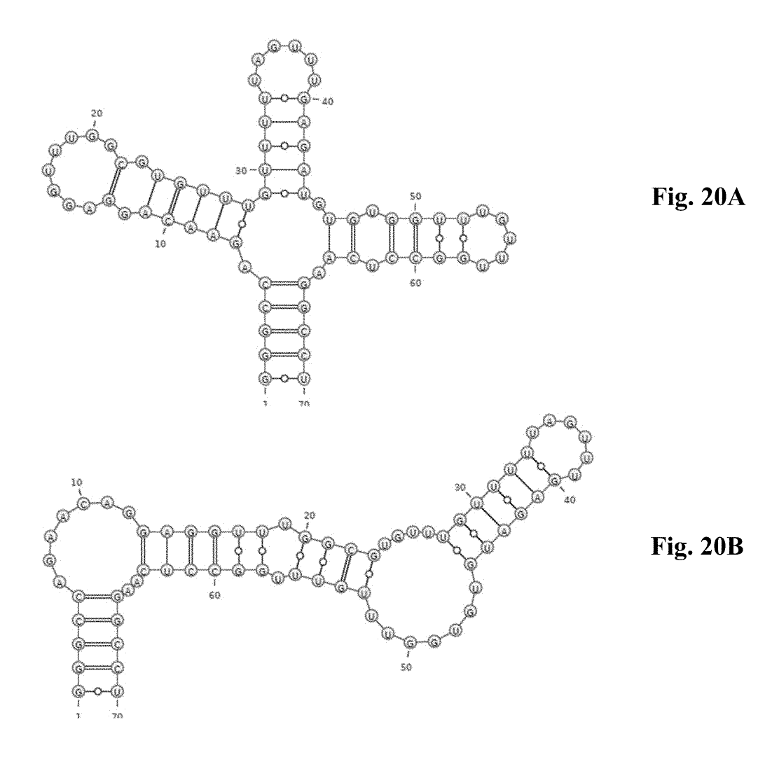

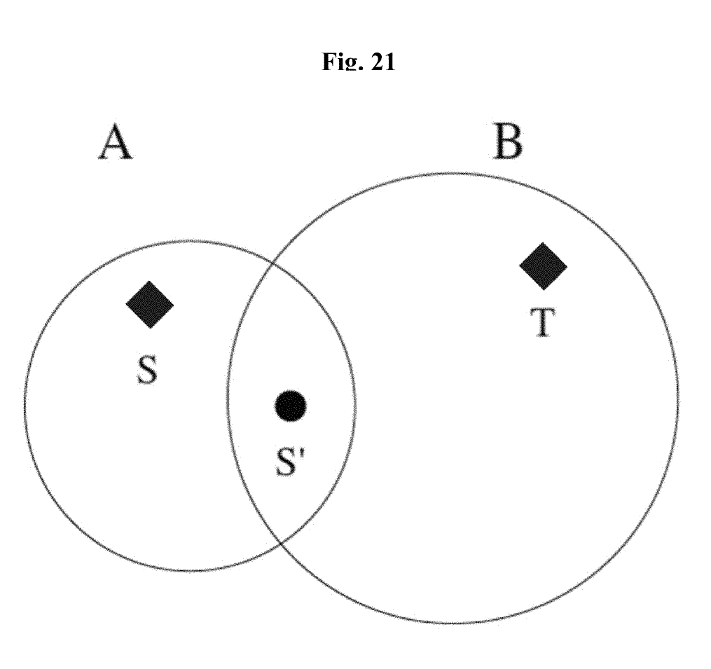

Employing the suboptimal structure generator in the Vienna RNA package we found that out of ten million random sequences of length 100, 29.5% have multiple structures at the MFE level. Among these sequences we found some having up to 144 distinct MFE-configurations. These degenerate sequences exhibit, as described above, different but somewhat similar MFE-structures, one of which is selected (at random) by the folding method or system as the MFE-structure. This multiplicity of phenotypes is a phenomenon that is observed among random and natural sequences alike. For example, 62% of more than ten thousand precursor miRNA sequences in the miRBase database (Kozomara and Griffiths-Jones, 2014) are predicted to have more than one structure at the MFE level by the Vienna RNA package. FIGS. 20A and 20B display two distinctively different MFE-structures of the precursor miRNA Bos Taurus miR-2405.

The abundant existence of MFE-sets of structures, rather than a unique MFE-structure, leads to considering extensions to our notions of distances between RNA secondary structures, such as base-pair distance, tree edit distance, Motzkin distance and others (Moulton et al., 2004). Such distances are widely employed to compare RNA sequences by comparing the distance between their MFE-structures and, in certain embodiments, we restrict the method to the base-pair distance. In some embodiments, we introduce a distance between sets of secondary structures, employing the mathematical notion of Hausdorff distance (not to be confused with the adaptation of Hausdorff distance as a secondary structure metric (Chen et al., 2011)), which is a classical min-max distance based on the above mentioned base-pair distance, see FIG. 21 and the disclosure below. In FIG. 21, A and B are the MFE sets of the degenerate sequences a and σ′, the diamonds represent the MFE structures chosen by the Vienna RNA package as MFEs. σ,σ′ are iNeutral but not neutral because they share an MFE structure but do not have identical MFE sets. In certain embodiments, we refer to this distance between sets of secondary structures as base-pair H-distance. Explicitly, if A, B are sets we set D(s,B)=min_{s′∈B}d(s, s′) and ^{\vec{}}D(A,B)=max_{s∈A}D(s,B). Note that ^{\vec{}}D(A,B)=max_{s∈A}D(s,B) and ^{\vec{}}D(B,A)=max_{s′∈B}D(s′,A) are not necessarily equal, hence we have to symmetrize D(A,B)=max{^{\vec{}}D(B,A), ^{\vec{}}D(B,A)} and D is a metric (Rockafellar and Wets Roger, 1998).

The multiplicity of MFE-structures and the implied extension to base-pair distances of sets of structures leads to a new notion of neutrality. Two adjacent sequences are neutral neighbors if their associated (MFE) structures are equal. Extending this notion, two adjacent sequences are intersection-neutral or iNeutral neighbors if their sets of MFE-structures have a nonempty intersection. While neutral neighbors are by construction iNeutral, two iNeutral neighbors may not be neutral: this happens when at least one of them is degenerate and the shared MFE-structure is not selected as the MFE-structure, see FIG. 21. However, as these two sequences have a common MFE-structure, it is reasonable, from a biophysical point of view, to consider them to be “neutral”, because all MFE phenotypes of a sequence have identical free energy.

We call two sequences iNeutral with regard to S if they both contain S as MFE-structure and, consequently, the iNeutral network of S is the set of all sequences that are iNeutral with regard to S. An immediate consequence of iNeutrality is that two iNeutral networks of two distinct structures, S and S′, intersect if and only if there exists one or more sequences that have S and S′ as MFE-structures. This is a novel feature of iNeutrality and in stark contrast to the induced partition of sequence space by neutral networks.

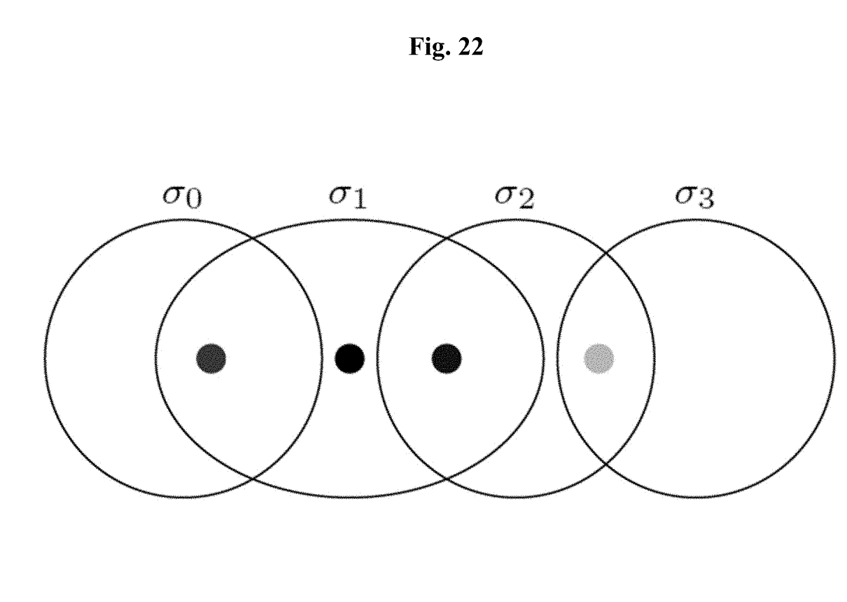

Equipped with the concept of iNeutrality, we can now naturally extend the notion of neutral walks (Schuster, 1994): an iNeutral walk, or drift walk is a sequence of RNA sequences σ₀, σ₁, . . . σₙ such that any two successive sequences, σᵢ,σᵢ₊₁ are iNeutral, see FIG. 22.

In FIG. 22, σ₀ and σ₃ are non-degenerate while σ₁ an σ₂ are degenerate. (Circles labeled by the sequences σᵢ represent MFE sets). Each consecutive pair of sequences, i.e., (σ₀,σ₁), (σ₁,σ₂), (σ₃,σ₄) is iNeutral because they share a MFE structure. Therefore, σ₁, σ₂, σ₃, σ₄ is a drift walk. Note that σ₀ and σ₃ are not iNeutral, but connected by a drift walk.

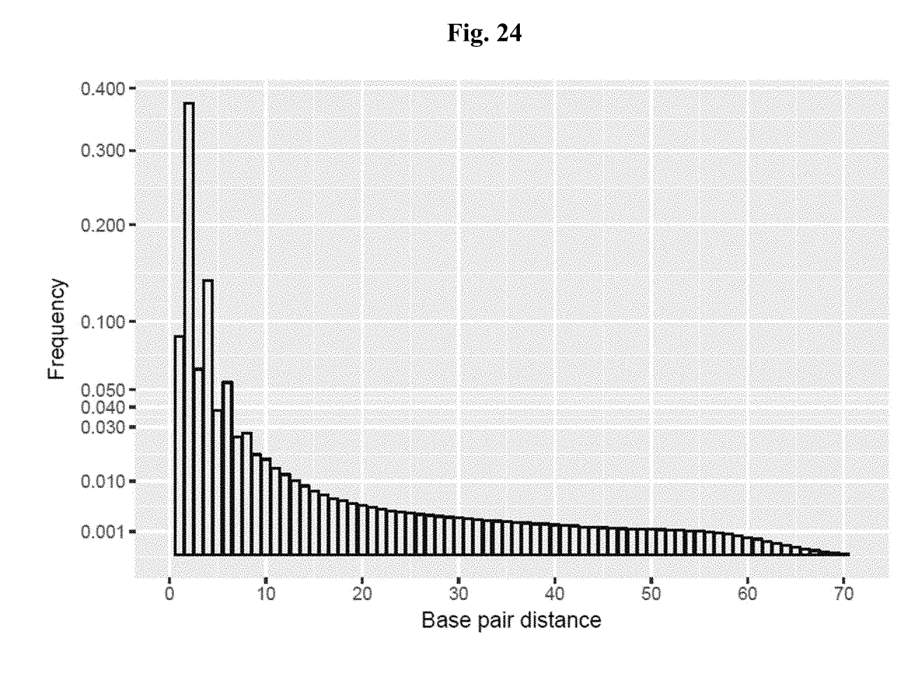

In the present disclosure, we demonstrate that drift walks, in analogy to neutral walks (Schuster et al., 1994), percolate sequence space. However, they do so at a much higher degree and the realized RNA secondary structures gradually change along such a walk. It turns out that drift walks, via accumulation of these gradual changes, produces massive changes in RNA structure, see FIG. 24. By construction, any structural change observed in drift walks is energy-neutral for a fixed sequence and as it is the case for neutral walks, energies may change as different sequences are traversed. The framework of drift walks differentiates between the interior and the boundary of neutral networks and we explore the properties of drift walks with respect to what amount of time they spend in either.

Drift walks are closely related to observed phenomena in evolutionary optimization, namely the existence of evolutionary relays (Fontana and Schuster, 1998), i.e., sequences of structures encountered in evolutionary optimization experiments in which two successive elements differ in specific ways. We show herein that there is evidence that drift walks can connect any pair of RNA secondary structures, which in turn means there exists a sequence of continuously changing structures connecting the starting and target structures. This observation explains the existence of evolutionary relays (Fontana and Schuster, 1998).

In embodiments, the present disclosure describes IFR (Equation 4) function as a feature of a sequence structure pair. IFR behaves differently among different species and between natural and random sequence structure pairs. By definition, IFR indicates the sequence capability of preserving phenotype. Hence, IFR of a sequence structure pair can be used to identify its robustness.

In embodiments, the MFE structures and base pairing probability are used to determine plasticity of a sequence structure pair.

The methods and systems described herein provide for the identification of functionally equivalent nucleic acid sequences that previously could not have been demonstrated or identified. Nucleic acid sequences that are functionally equivalent have the same or similar functional activity or biological activity. As an example, the functional activity of a nucleic acid sequence is inhibiting the expression of a gene. A second nucleic acid sequence having the same functional activity as a first nucleic acid can inhibit the expression of the same gene to the same extent (100%) as the first nucleic acid sequence. A second nucleic acid sequence having a similar functional activity can inhibit the expression of the same gene by at least about 90% as compared to the the first nucleic acid sequence.

In some embodiments, the systems and methods enable structural comparison and identification of similarity or lack thereof between two or more distinct nucleic acid sequences, which comparison was not previously known or possible. Such methods and systems allow for the identification of distinct nucleic acid molecules that are similar on a structural level but that would be deemed distinct, i.e., not similar on a sequence level, by previously known methods (e.g., BLAST). Conversely, in embodiments, the methods and systems described herein allow for the identification of distinct secondary structures of nucleic acid molecules, which structures are similar on a sequence level, i.e., can arise from the same sequence, but that would be deemed structurally distinct, i.e., not similar on a structural level, by previously known methods. As a result, the systems and methods described herein enable efficient and powerful pairing between genotypic (sequence) and phenotypic (structure and function) information not previously possible or available.

In embodiments, the methods described herein can be implemented using a computer. The computer includes a processing unit such as a CPU or DSP, and instructions executable by the processor to cause the processor to perform functions or steps described herein. The processing unit can include a processing unit can include an ASIC, FPGA, or other logic device(s) wired (e.g., physically, via blown fuses, or via logic-cell configuration data) to perform functions described herein. In embodiments, the computer includes memory, one or more processors, and computer readable media.

The computer readable media can be or include computer storage media. Computer storage media can include, but are not limited to, random-access memory (RAM), static random-access memory (SRAM), dynamic random-access memory (DRAM), phase change memory (PRAM), read-only memory (ROM), erasable programmable read-only memory (EPROM), electrically erasable programmable read-only memory (EEPROM), flash memory, compact disc read-only memory (CD-ROM), digital versatile disks (DVDs), optical cards or other optical storage media, magnetic cassettes, magnetic tape, magnetic disk storage, magnetic cards or other magnetic storage devices or media, solid-state memory devices, storage arrays, network attached storage, storage area networks, hosted computer storage or memories, storage, devices, or any other tangible, non-transitory medium which can be used to store the desired information and which can be accessed by the processor. Tangible computer-readable media can include volatile and nonvolatile, removable and non-removable media implemented in any method or technology for storage of information, such as computer readable instructions, data structures, program modules, or other data. In contrast to computer storage media, computer communication media can embody computer-readable instructions, data structures, program modules, or other data in a modulated data signal, such as a carrier wave, or other transmission mechanism. As defined herein, computer storage media does not include computer communication media.

The methods and systems described herein are useful, for example, in characterizing, identifying, sampling, and determining secondary structures of nucleic acids. Such methods and systems can include determining relatedness of nucleic acid sequences based on probable structures, as well as determining structural relatedness based on probable sequences. The methods and systems are useful in the context of determining and characterizing the potential function of nucleic acid molecules (e.g., any function associated with the secondary structure of the nucleic acid molecule) both in healthy and diseased organisms, tissues, and/or cells. Such scenarios may include, but are not limited to, cancer and known genetic disorders. Additional applications of the systems and methods described herein include, but are not limited to, toxicology, mutational analysis, forensic analysis, precision or personal medicine, design of vaccines, study of the microbiome, and so forth.

The following exemplary embodiments and examples illustrate exemplary methods provided herein. These exemplary embodiments and examples are not intended, nor are they to be construed, as limiting the scope of the disclosure. It will be clear that the methods can be practiced otherwise than as particularly described herein. Numerous modifications and variations are possible in view of the teachings herein and, therefore, are within the scope of the disclosure.

### EXEMPLARY EMBODIMENTS

The following are exemplary embodiments.

E1. A method of characterizing a nucleic acid sequence, the method comprising: determining robustness of the nucleic acid sequence based on an inverse fold rate of a sequence structure pair at a predetermined hamming distance associated with the nucleic acid sequence; and determining plasticity of the genetic sequence based on base pairing probability.

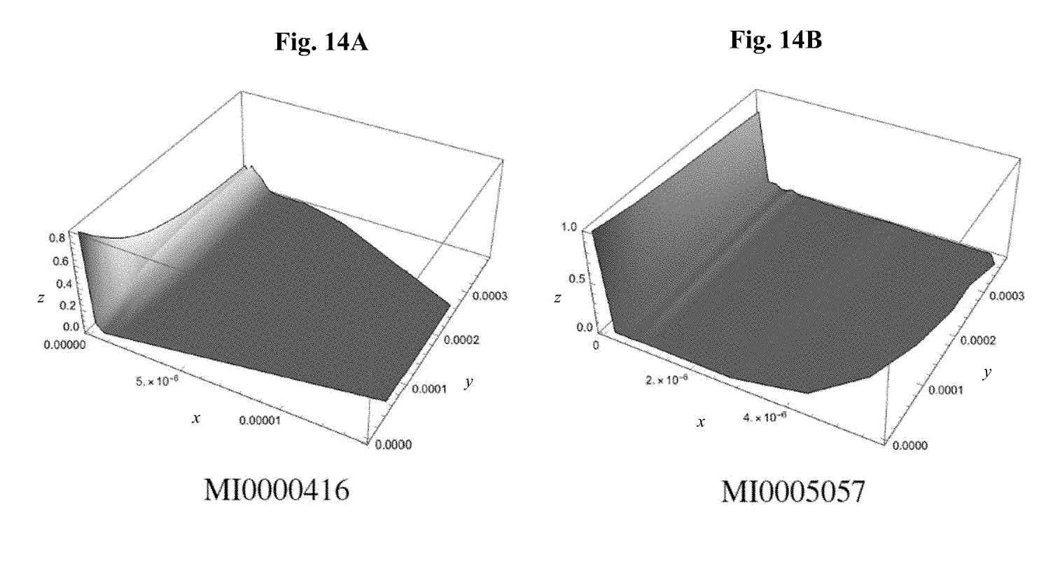

Mutations are energy biased instead of uniformly selected. We consider a discrete structural distance instead of the BP distance. Also, we can consider multiple point-mutations instead of just single point-mutations. Robustness is defined via IFR comparison with random sequences that fold into the same structure. If the IFR of the native pair is higher than the mean of random pair then it is robust. Plasticity can be defined similarly. The cross-species difference of robustness and plasticity manifests in higher level organisms having higher levels of robustness. See FIG. 14,15.

E2. A method of identifying a set of nucleic acid sequences that form desired compatible structures, the method comprising: generating a first plurality nucleic acid sequences; using a partition function sampling (Equations 6 and 7) from the first plurality of nucleic acid sequences to obtain a second plurality of nucleic acid sequences; and identifying from the second plurality of nucleic acid sequences a set of nucleic acid sequences that form the desired compatible structures; wherein the desired compatible structures are functionally equivalent to a specific structure associated with a specific biologic function.

In embodiments, Boltzmann sampling instead of random walks are used to obtain compatible sequences and hamming/double filtration is used to study regions of interest. A structure is compatible to a sequence if all its base pairs are canonical, namely, AU, GC and GU. In embodiments, the first plurality of sequences (background) are obtained using dual sampling with no restriction, and the second plurality of sequences is obtained by Boltzmann sampling with Hamming filtration/double filtration. In embodiments, we can have various notions of functional equivalence. Namely the structures are exactly the same or vary by some degree without affecting their functional activity. The BP distance is small. It can be defined with respect to certain fixed substructures that are the same.

E3. The method of embodiment E2, wherein the generated first plurality of nucleic acid sequences has a predefined energy threshold. Low energy sequence-structure pairs are thermodynamically more stable. Thus, we can only sample suboptimal sequences. i.e., we only sample sequences within a certain energy bandwidth around the energy of the MFE sequence.

E4. The method of any one of embodiments E2-E3, wherein the identified set of nucleic acid sequences includes progressively differing nucleotide sequences forming the desired compatible structures. This is motivated by the notion of neutral evolution. Progressively differing means there is a path formed by sequences of NA where consecutive NAs differ by 1 or 2 nucleotides and all NAs fold into the same structure. Prior to the present discovery, such a path is constructed using exhaustive searches. Our method is efficient and works quite well for long Hamming distances.

E5. The method of any one of embodiments E2-E4, wherein the desired compatible structures are formed by the identified set of nucleic acid sequences at a predefined energy threshold. As explained under embodiment E3, low energy sequence-structure pairs are thermodynamically more stable. Thus, we can only sample suboptimal sequences. i.e., we only sample sequences within a certain energy bandwidth around the energy of the MFE sequence.

E6. The method of any one of embodiments E2-E5, wherein the desired compatible structures formed by the identified set of the nucleic acid sequences are not identical to each other. We introduce this extra restriction in order to obtain a representative sequence in each structural class. So, they have the potential to perform different function.

E7. The method of any one of embodiments E2-E6, wherein energy states of the desired compatible structures formed by the plurality of nucleic acid structures in the identified set differ. In embodiments, energy states are just the thermodynamic energy given by the pairing. This extra restriction can be introduced in order to obtain a representative sequence in each energy class, as well as to obtain a sequence spectrum of thermodynamic stability.

E8. The method of any one of embodiments E2-E7, wherein the identified set progressively increases in distance from a desired structure, the distance being based on base-pair H-distances. In embodiments, the structures form a path such that the distance to the original structure is increasing by 1 or 2 at each step, and we sample with double filtration and increasing BP distance since this allows us to mimic continuous evolution.

E9. The method of embodiment E8, wherein the specific structure has a specific nucleotide sequence; wherein a first nucleic acid sequence in the identified set has a nucleotide sequence more distinguishable from the specific nucleotide sequence than a second nucleic acid sequence in the identified set having a lesser distance from the desired structure. In embodiments, more distinguishable refers to a sequence with larger Hamming distance forms structures with larger BP distance. These sequences can be obtained using the sampler with double filtration. This allows us to study continuous evolution.

E10. The method of any one of embodiments E2-E9, wherein at least some of the desired compatible structures are identical.

E11. The method of any one of embodiments E2-E10, wherein the predefined energy threshold is a minimum free energy threshold. In embodiments, when the energy threshold is 0 in E3, all the minimum free energy (MFE) structures are considered. Note that the minimum free energy structure might not be unique. There is the degeneracy in the MFE map.

E12. A method of identifying a set of nucleic acid sequences that form a specific structure, the method comprising: generating an initial population of nucleic acid sequences that form the specific structure; using each of the nucleic acid sequences in the initial population of nucleic acid sequences to generate a first plurality of nucleic acid sequences from each of the nucleic acid sequences in the initial population; using a partition function sampling (Equations 6 and 7), from the first plurality of nucleic acid sequences to obtain a second plurality of nucleic acid sequences; and identifying from the second plurality of nucleic acid sequences a set of nucleic acid sequences that form the specific structure; wherein the specific structure formed by the identified set of nucleic acid sequences is functionally equivalent to a desired specific structure associated with a specific biologic function.

Mutation is biased towards thermodynamic stability instead of being uniformly random. The sequence to structure map is not one to one. The multiplicity of MFEs is considered, i.e. the multiple structures that all achieve the minimum energy. In embodiments, for generating an initial population of nucleic acid sequences, Boltzmann sampling is used to imitate mutation and create descendant sequences. Then, the multiplicity of MFEs is considered. This models the quasi-neutral evolution.

E13. The method of embodiments E12, wherein the specific structure is formed by the generated initial population of nucleic acid sequences that are identical.

E14. The method of embodiment E12 or E13, wherein the specific structure is formed by the generated initial population of nucleic acid sequences formed at a predefined energy threshold. Please also see E3.

E15. The method of any one of embodiments E12-E14, wherein the generated first plurality of nucleic acid sequences has a predefined energy threshold. Please also see E3.

E16. The method of any one of embodiments E12-E15, wherein the identified set of nucleic acid sequences includes progressively differing nucleotide sequences forming the specific structure. Please also see E4.

E17. The method of any one of embodiments E12-E16, wherein the specific structures formed by the identified set of the nucleic acid sequences are not identical to each other.

E18. The method of any one of embodiments E12-E17, wherein energy states of the specific structures formed by the plurality of nucleic acid structures in the identified set differ. Please also see E7.

E19. The method of any one of embodiments E12-E18, wherein the identified set of nucleic acid sequences progressively increases in distance from the specific structure, the distance being based on base-pair H-distances. Please also see E8.

E20. The method of any one of embodiments E12-E19, wherein the specific structure has a specific nucleotide sequence; wherein a first nucleic acid sequence in the identified set has a nucleotide sequence more distinguishable from the specific nucleotide sequence than a second nucleic acid sequence in the identified set having a lesser distance from the desired structure. Please also see E9.

E21. The method of any one of embodiments E12-E20, wherein the predefined energy threshold is a minimum free energy threshold. Please also see E11.

E22. A method of determining similarity of two or more nucleic acid sequences, the method comprising: identifying the one or more secondary structures formed by an initial nucleic acid sequence; generating a plurality of nucleic acid sequences that form the identified one or more secondary structures formed by the initial nucleic acid sequence; and identifying at least one nucleic acid sequence from the plurality of nucleic acid sequences, wherein the similarity of the initial nucleic acid and the at least one nucleic acid sequence is based on the identified one or more secondary structures.

Prior to the present discovery, sequence similarity was mostly measured by the similarity of nucleotide sequences through alignment. The present disclosure describes a novel method of determining analyzing sequence similarity which is based on the duality of sequence and structure and involves computation including probability. In embodiments, two sequences are similar if their structural ensemble and the structure ensemble of their mutations are similar. For each sequence, we can compute or sample from its structure ensemble. Furthermore, we can compute or sample from the structure ensemble of all its mutants, using our sampler with Hamming filtration. Then we can compare those structures using a given structure distance and obtain a similarity score.

E23. The method of embodiment E22, wherein the identified at least one nucleic acid sequence has a predefined energy threshold. Please also see E4.

E24. The method of any one of embodiments E22-E23, wherein the identified at least one nucleic acid sequence includes two or more nucleic acid sequences, the two or more nucleic acid sequences have progressively differing nucleotide sequences and form the desired compatible structures. Please also see E4.

E25. The method of any one of embodiments E22-E24, wherein the identified one or more secondary structures are formed by the identified one more nucleic acid sequences at a predefined energy threshold. Please also see E5.

E26. The method of any one of embodiments E22-E25, wherein the predefined energy threshold is a minimum free energy threshold. Please also see E11.

E27. A method of determining similarity of one or more secondary structures of nucleic acid sequences, the method comprising: identifying at least one initial secondary structure; generating a first plurality of nucleic acid sequences that form the identified at least one secondary structure at a first predetermined energy threshold; using a partition function, sampling from the first plurality of nucleic acid sequences to obtain a second plurality of nucleic acid sequences; and determining a set of secondary structures formed by the second plurality of nucleic acid sequences at a second predetermined energy threshold; wherein the similarity of the at least one initial secondary structure and the set of secondary structures is based on at least one of the first and second plurality of nucleic acid sequences.

In embodiments, this is the dual of E22. Two structures are similar if their sequence ensemble and the sequence ensemble of their neighbors are similar. This can be defined similarly as E22, and can be computed using Boltzmann partition function with double filtration.

E28. The method of embodiment E27, wherein the first predefined energy threshold and the second predefined energy threshold are the same. Please also see E3.

E29. The method of any one of embodiments E27-E28, wherein at least one of the first and second predefined energy thresholds are a minimum free energy threshold. Please also see E11.

E30. A method of identifying functionally equivalent nucleic acid sequences, wherein the method comprises identifying a specific functional activity; identifying a nucleic acid structure associated with the functional activity; identifying nucleic acid sequences associated with the nucleic acid structure, thereby identifying functionally equivalent nucleic acid sequences. In embodiments, identifying nucleic acid sequences associated with the nucleic acid structure comprises first identifying a plurality compatible sequences using inverse folding algorithm or dual sampling; for each sequence, computing IFR using Boltzmann sampler with Hamming distance filtration, and then, identifying sequences with similar IFR (within 10% relative difference).

E31. A method of identifying a plurality of secondary structures for a nucleic acid sequence, wherein the method comprises obtaining a nucleic acid sequence; determining the robustness and plasticity of the nuclei acid sequence; identifying a plurality of secondary structures having similar robustness and plasticity, thereby identifying a plurality of secondary structures for the nucleic acid sequence. In embodiments, the method comprises identifying a plurality of secondary structures having similar robustness and plasticity via Boltzmann Sampling with Hamming distance filtration (within 10% relative difference).

E32. The method of embodiment 31, wherein the plurality of secondary structures comprises an equivalent functional or biological activity.

E33. A method of identifying a plurality of nucleic acid sequences for a secondary structure, wherein the method comprises identifying a secondary structure; determining a nucleic acid sequence for the secondary structure; determining the robustness and plasticity of the nucleic acid sequence; identifying nucleic acid sequences having similar robustness and plasticity as the nucleic acid sequence, thereby identifying a plurality of sequences for the secondary structure. In embodiments, the method comprises identifying a plurality of nucleic acid sequences having similar robustness and plasticity via Boltzmann Sampling with Hamming distance filtration (within 10% relative difference).

E34. The method of embodiment 33, wherein the plurality of nucleic acid sequences comprises an equivalent function or biological activity.

E35. The method of any one of embodiments E1-E34, wherein the method is implemented using a computer, wherein the computer includes one or more processors and a memory.

### EXAMPLES

### Example 1

Experimental Design.

Data presented in Example 1 is related to (miRNA) precursor in the let-7 family. miRNA precursor is a class of ncRNAs that fold into stem-loop structures. In addition, the stem-loop structures are highly conserved, which contributes to the attractiveness of the molecule for studying shape modulated evolution (Mimouni, et al., 2008). In particular, the let-7 family has been widely detected in metazoans. Provided herein is a comparison of natural sequence structure pairs with artificial sequence structure pairs, as well as an evolutionary analysis of let-7 sequences across different species using the methods and systems described herein.

Sequences in the let-7 gene family, including miRNAs from different species, were obtained from miRBase database. The MFE structures of the molecules were computed to create sequence structure pairs. For each sequence structure pair, the IFR and BP probability were calculated at hamming distance 1≤k≤20 by sampling 50000 sequences.

For each species, 3 structures were randomly selected, and for each structure, 5 sequences were randomly generated using RNAinverse. For each sequence structure pair, the IFR and BP probability were calculated at hamming distance 1≤k≤20 by sampling 50000 sequences.

### Results.

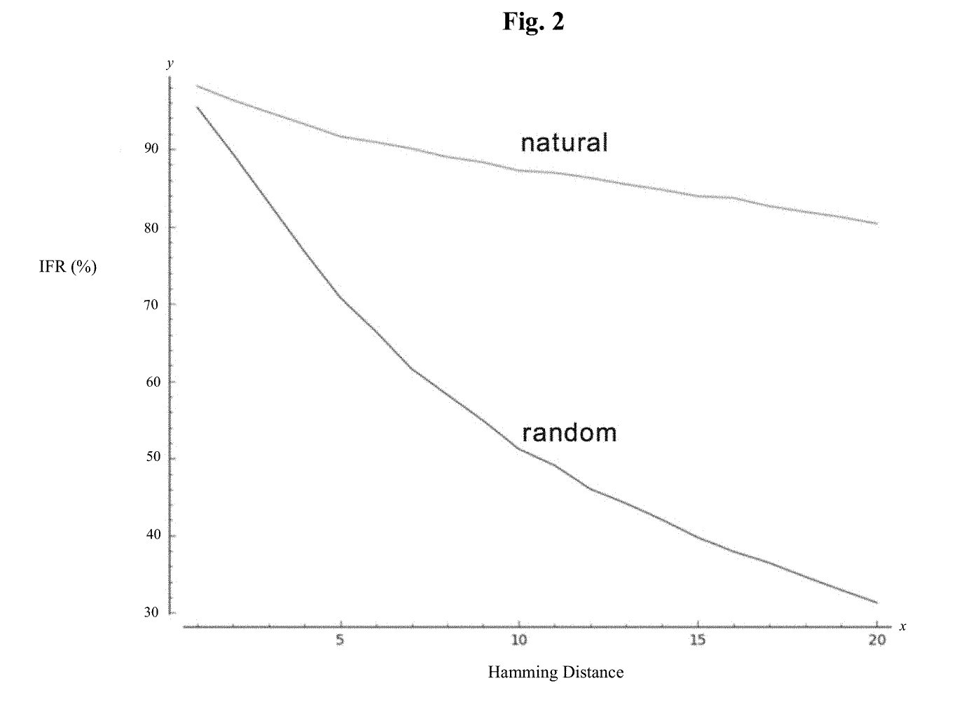

Data from the IFR of the stem-loop structure corresponding to MI0005062, from 1≤k≤20, is presented in FIG. 2, where the green curve represents the IFR of the natural sequence structure pair. The Red curve represents the average IFR of 5 random sequences that fold into the same structure.

By comparing the IFR between natural and random sequences that fold into the same structure, it is observed that natural sequence structure pairs exhibit higher IFR. The difference becomes very significant at large hamming distances. As seen in FIG. 2, at k=1, IFR corresponding to natural pairs is slightly higher than random pairs. As hamming distance k increasing from 1 to 20, both curves decrease, but IFR corresponding to natural pairs decreases much slower, and that leads to the significant difference at k=20. This result is consistent with the findings of Borenstein (Borenstein and Ruppin, 2006), however, our sampling method allows us to look at much larger hamming distance, where the differences between natural and random pairs becomes more significant.

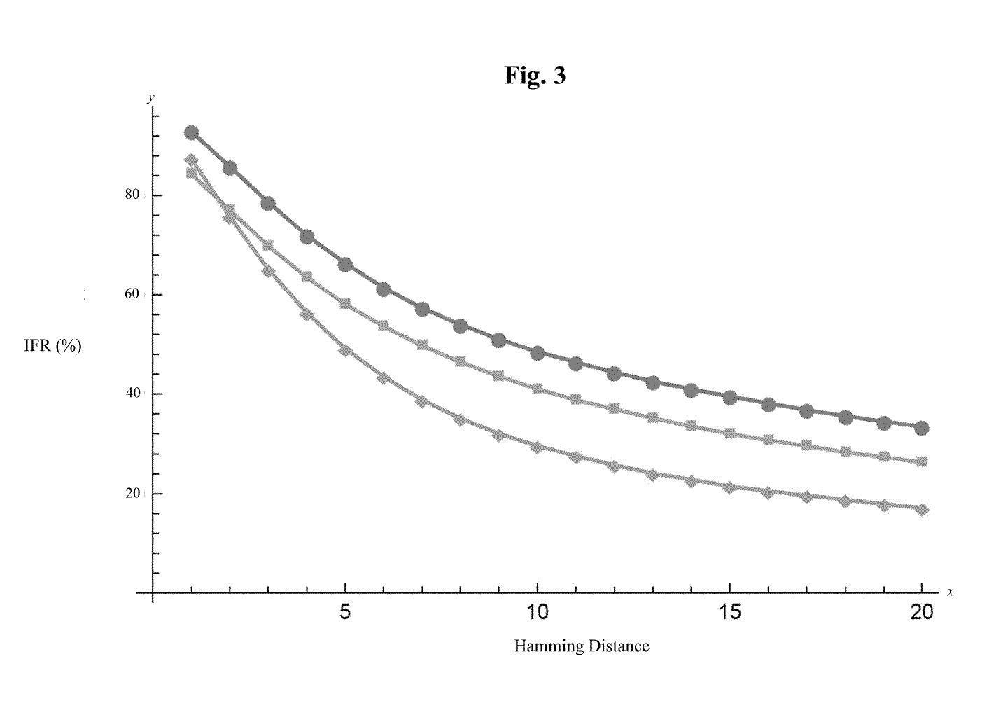

High metazoans have higher IFR. IFR across species was compared. FIG. 3 provides data for IFR (mean) of sampled sequences from the natural structure of microRNA let-7 family of three classes: human (circle), lizard (square) and drosophila (diamond). The length of sequences ranges from 80 nts to 120 nts, with a sample size of 50000. Human has the highest IFR while Drosophila has the lowest, which is consistent with our understanding about robustness and complexity.

Plasticity.

Plasticity has previously been used to describe the structural variance of the Boltzmann structure ensemble of a single RNA sequence. Herein, instead of considering a single sequence, the structural variance of the MFE structures of the sampled sequences were determined. Essentially, this is a type of mutational plasticity, directed to the potential of structural evolution of a sequence structure pair against (Boltzmann weighted) mutations.

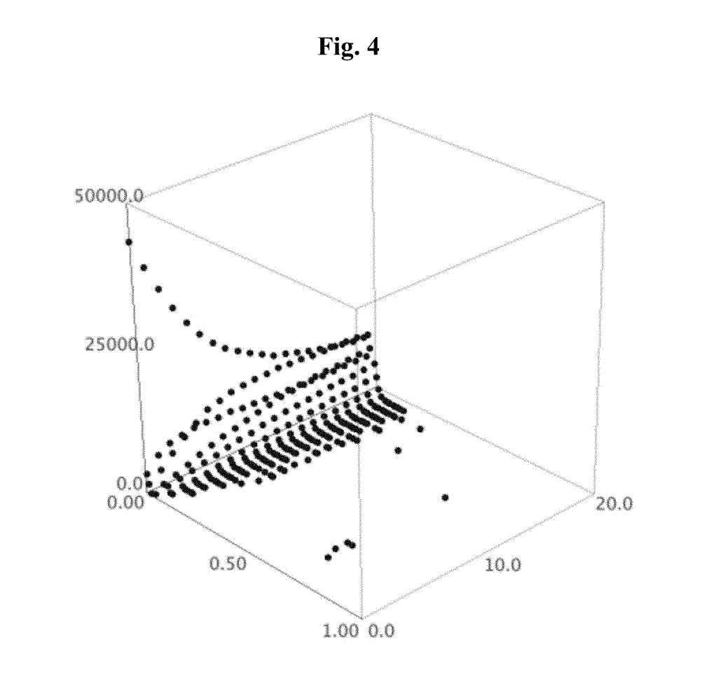

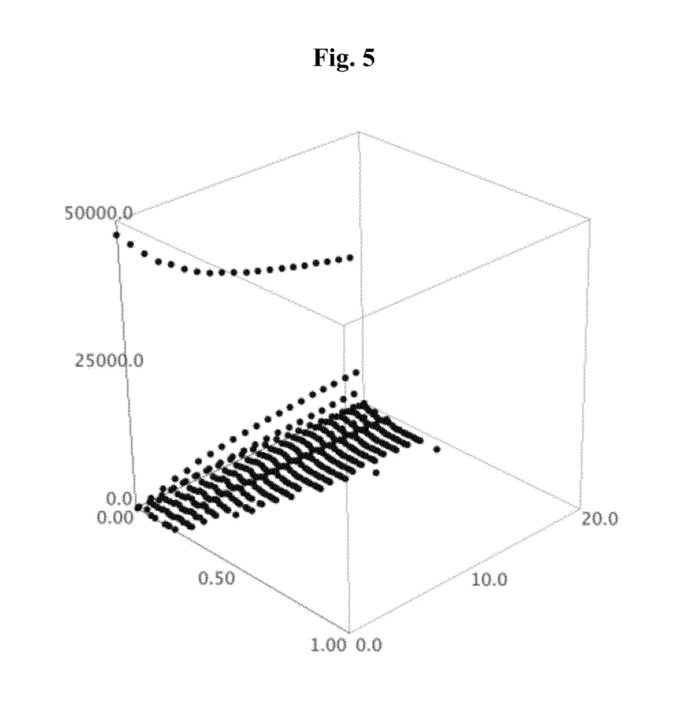

First, we considered the MFE structures of the sampled sequences and their structural distance to the reference structure, using base pair distance as our structural distance. The distribution of the base pair distance of the MFE structures of the sampled sequences to the reference structure, corresponding to MI0000416 and MI0005057 are displayed in FIGS. 4 and 5, respectively.

In FIG. 4, distribution of base pair distance between the MFE structure of sample sequence and the reference structure of MI0000416 are shown. The x-axis denotes the relative base pair distance, the y-axis denotes the hamming distance, and the z axis denotes the frequency. In FIG. 5, distribution of base pair distance between the MFE structure of sample sequence and the reference structure of MI0005057 are shown. The x-axis denotes the relative base pair distance, the y-axis denotes the hamming distance, and the z-axis denotes the frequency.

MI0000416 (FIG. 4) is a Drosophila miRNA while MI0005057 (FIG. 5) is a cattle miRNA. In both figures, the data demonstrates that the inverse fold rate decreases as hamming distance increases (corresponding to x=0). But IFR decreases in MI0005057 (FIG. 5) much slower than in MI000416 (FIG. 4). Moreover, in MI000416, the MFE structures of the sampled sequence are clustered around the reference structure. While in MI005057, a sharp decrease in frequency after x=0 is observed. There are much fewer structures at small but nonzero distance from the reference structure in MI005057 than that in MI000416. The distance distribution in the data for MI005057 is even more spread out than that in MI000416. This indicates that the high IFR in cattle is not obtained by sacrificing the ability to switch to distant structures (structures that have truly distinct configurations). Sampled sequences in MI0005057 either try to preserve the given structure, or fold to a really different structure. It is more likely to be achieved by ‘absorbing’ nearby structures (some kind of local optimal).

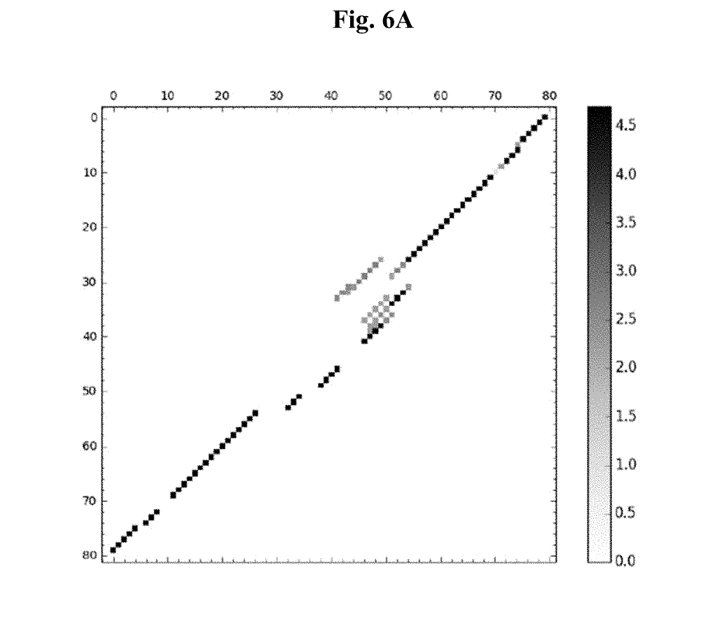

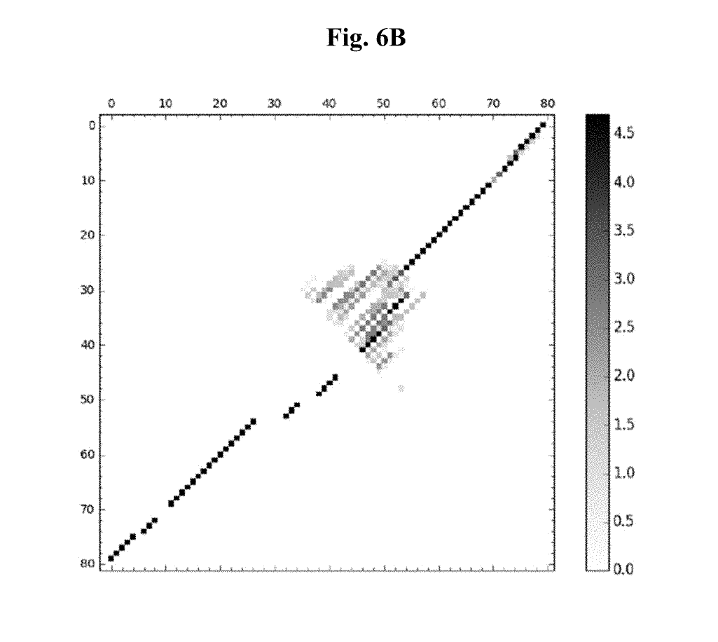

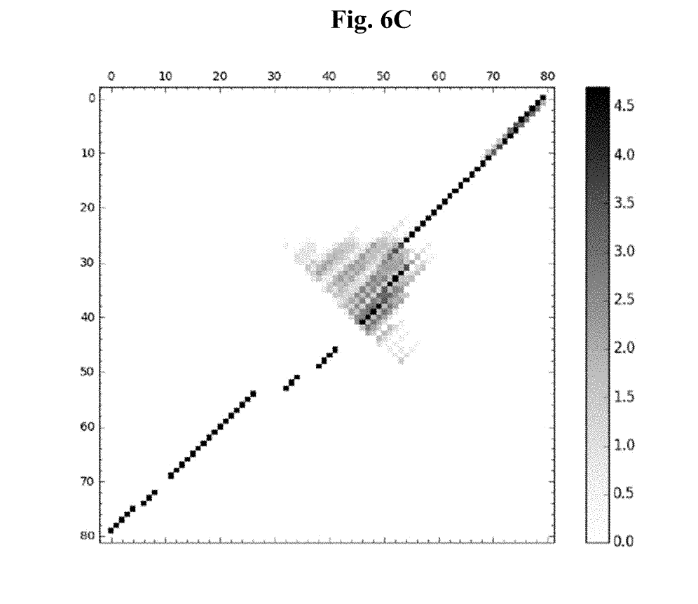

FIGS. 6A-C present data from an alternative approach to studying the structural variance of the ensemble of MFE structures of the sampled sequences. In this case, the base pairing probability matrix for the ensemble of MFE structures of the sampled sequences for each sequence structure pair at each hamming distance was computed. In FIGS. 6A-C, base pairing probability of MI0005057 at different hamming distances is shown. The upper right part of each matrix shows the BP probability, while the lower left half shows the original structure. In FIG. 6A k=1, in FIG. 6B k=5, and in FIG. 6C k=20.

As the hamming distance increases, more and more configurations pop up in the ensemble. This can be observed from the increasing ‘fuzziness’ in the base pairing probability matrix. Furthermore, the fuzziness is mainly observed at the loop region and a little bit at the beginning of the stem loop region. The middle part of the stem is very stable, which is exactly where the mature miRNA lies.

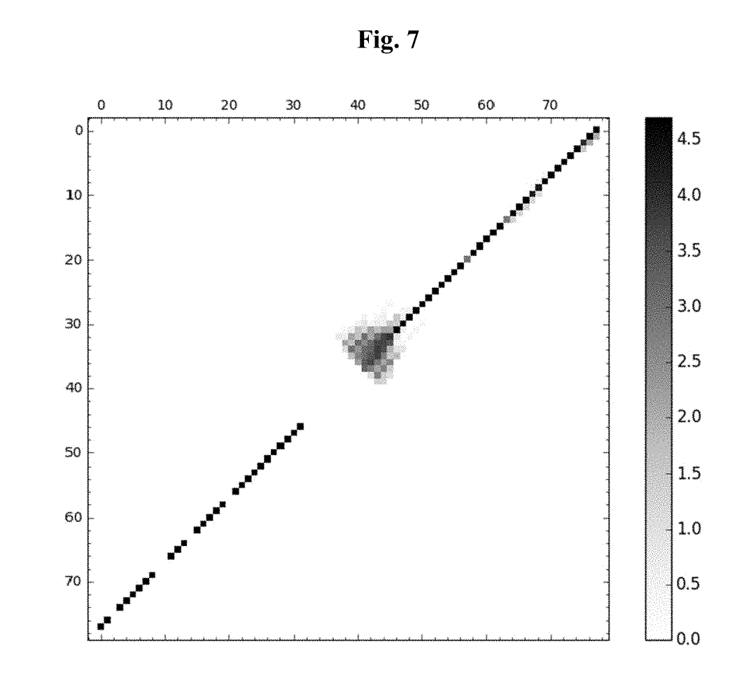

FIG. 7 shows base pairing frequency of MI0000416 (from drosophila) at h=20. The upper right half corresponds to the base paring frequency, while the lower left part corresponds to the reference structure. A comparison of FIG. 6C and FIG. 7 shows that there is more fuzziness in the cattle image compared to drosophila. This indicates that cattle has greater structural variation in its ensemble than that in drosophila.

A miRNA with Extremely Low IFR.

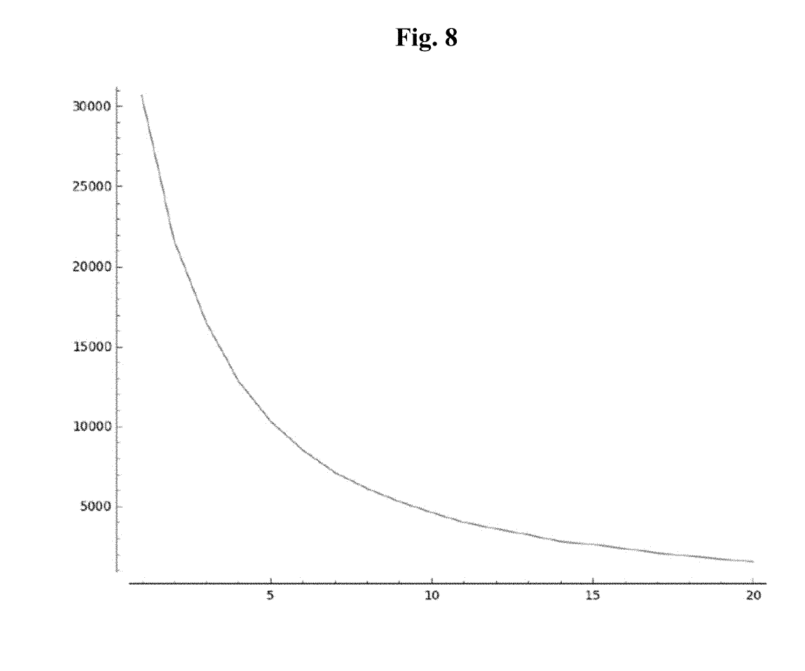

Most of the natural sequence structure pairs exhibit high IFR, however, we also observed a natural miRNA with extremely low IFR. MI0015952 is a cattle let-7 miRNA. As demonstrated in FIG. 8, this miRNA has extremely low IFR, indicating it is not robust at all. FIG. 8 shows the IFR of MI0015952 from 1 k 20. At k=20, IFR is around 2% while the average IFR for cattle is around 40%. Interestingly, its extremely low read is demonstrated in deep sequencing results (18.3 RPM compare to hundred or thousand RPM). Without being bound to a particular theory, it is suspected that the low read comes from the fact that it is being eliminated through selection due to being so un-robust.

### Example 2

Hamming Distance Distribution.

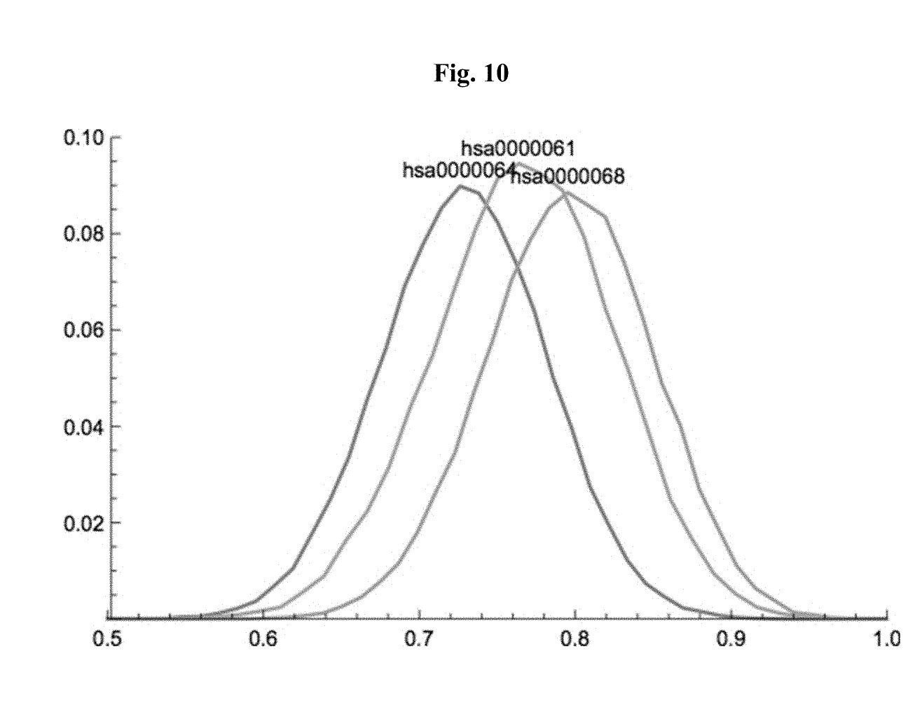

As mentioned above, a simple rejection sampler does not work as the number of sequences in each distance class is highly biased. We selected 12 sequence structure pairs of the human microRNA let-7 family. For each pair, we sampled 50,000 sequences using the unrestricted sequences sampler. We computed the hamming distance distribution to the natural sequence of the sampled sequences, and then normalized the distribution by sequence length, see FIG. 10.

In FIG. 10, sequence distribution of sampled sequences by Boltzmann sampler without distance filtration with respect to their hamming distance to the natural sequence are shown. The distance was normalized by the sequence length. The data are for microRNA let-7 family (human): hsa0000064, hsa0000068 and hsa0000061.

The data demonstrate that the most sampled sequences have distances of 70% to 90% of the sequence length away from the reference, while there is hardly any sequence being sampled outside this range. Similar results are obtained when replacing the natural sequence by a random sequence that folds to the structure. This implies that the rejection sampler on the old Boltzmann sequence cannot produce the full spectrum of distance classes from the reference sequence. Hence, our disclosed filtration method is superior and necessary.

Inverse Fold Rate.

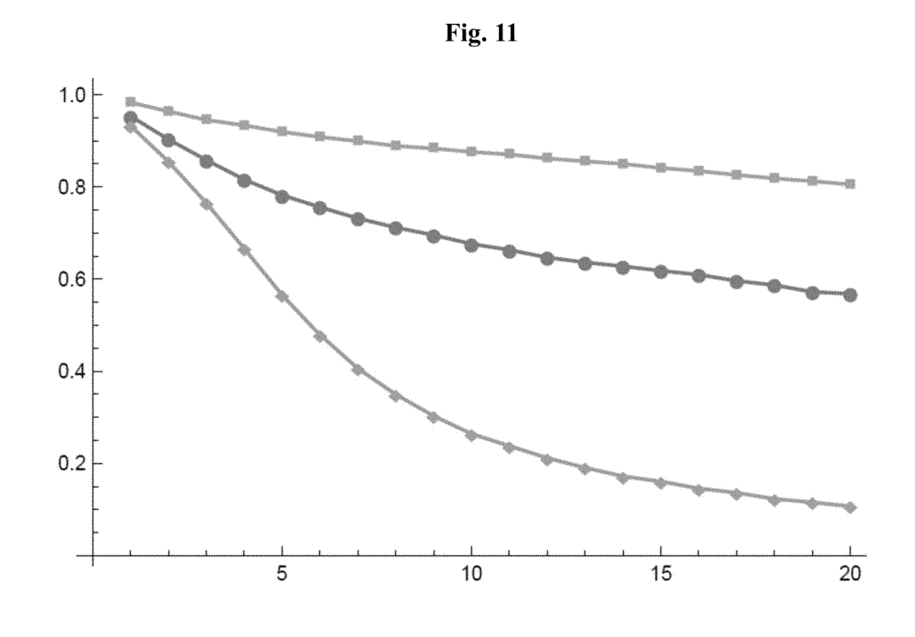

Next, we determined the inverse folding rate (IFR) of the sampled sequences with respect to different sequence structure pairs, as well as different hamming distances. We associated a 0-1 random variable to each sampled sequence: it takes value 1 if the sequence actually folded into the reference structure and takes value 0 otherwise. IFR is the mean of this random variable. Then, we can define the IFR of a sequence structure pair as a function of hamming distances to the reference sequence. We find that the IFR varies from pairs to pairs, see FIG. 11. FIG. 11 demonstrates that neighborhood IFR varies from different sequence structure pairs. FIG. 11 displays the IFR as a function of distance of three sequence structure pairs from human microRNA let-7 family: hsa0000060 (circles, 80 nts), hsa0000067 (squares, 87 nts) and hsa0000100 (diamonds, 119 nts). The sample size was 50000. The three sequences were selected with IFR dropping slowest, mediocre, and fastest when distance increases. Although hsa0000100 is the longest sequence among the three, the IFR drops much faster than the others.

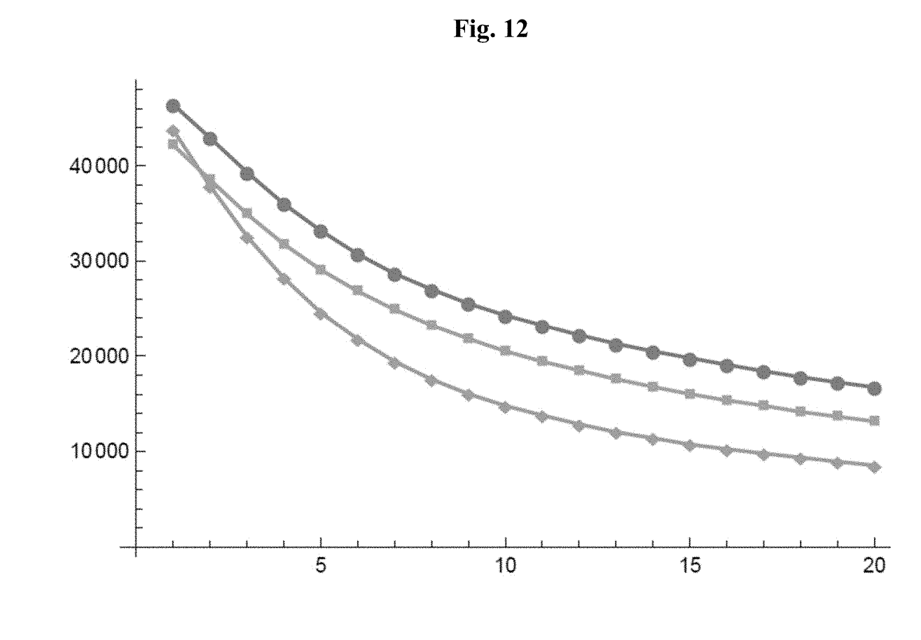

We also compared IFR across species. We collected all sequence structure pairs in the microRNA let-7 family from three species: human, lizard, and drosophila. Then we computed the mean IFR of each species, see FIG. 12. FIG. 12 shows IFR (mean) of sampled sequences from the natural structure of microRNA let-7 family of three classes: human (circles), lizard (squares) and drosophila (diamonds). The length of sequences ranges from 80 nts to 120 nts, with a sample size of 50,000. Human has the highest IFR while Drosophila has the lowest, which is consistent with our understanding about robustness and complexity.

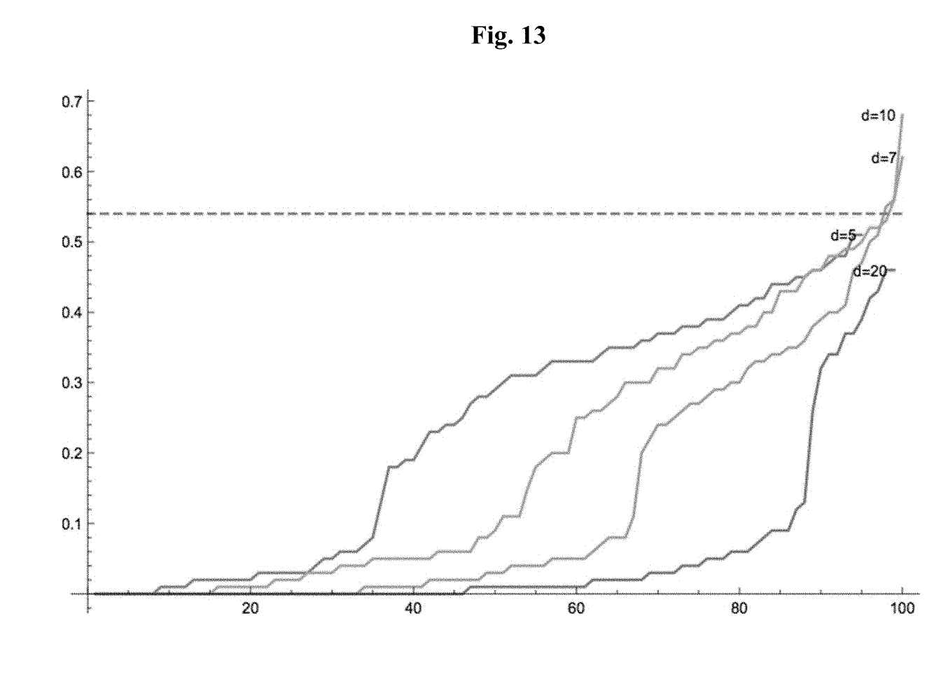

In addition, we compared the IFR of random sequence structure pairs (first find a random sequence then fold to structure) and natural ones. We used a random sequence of length 80 nt, the IFR is much lower than the natural one (drops to zero at distance 20). Motivated by this result, we then conducted a more detailed study of the IFR of natural sequence structure pairs. We created 100 sequence structure pairs by sampling sequences from the natural pairs with distances 5, 7, 10 and 20, respectively. Then we looked at the IFR of the new pairs at distance 5. We sorted the pairs by their IFR in increasing order and displayed them in FIG. 13. In FIG. 13, IFR of natural sequence structure pairs are displayed versus its neighbor sequences. We used the natural sequence structure pair of hsa0000063 and sampled 100 sequences with distance filtration 5, 7, 10 and 20, respectively. We paired each sampled sequence with the origin structure to create new sequence structure pairs. We looked at the IFR at distance 5 and sorted the sequences by their IFR in increasing order. We displayed the sequence IFR (classified by the distance of sampled sequence to the origin: distances of 5, 7, 10, and 20). The dashed line is the IFR of the natural pair at distance 5. We also displayed the IFR at distance 5 of the natural pair (dashed line).

The IFR of natural pair at distance 5 is above the 95% rank, which is better than the sampled pairs. Furthermore, IFR drops significantly when below 0.3. This implies that the sampled pairs have either a high IFR (>0.3) or a low IFR (<0.1). There are very few between them. The proportion of sequences having high a IFR drops when the distance to the natural sequence increases. This implies the natural pair is almost local optimal.

As shown herein, IFR function is a feature of a sequence structure pair. IFR behaves differently among different species and between natural and random sequence structure pairs. By definition, IFR indicates the sequence capability of preserving phenotype. Hence, IFR of a sequence structure pair can be used to identify its robustness.

Structural Diversity.

We have demonstrated that the sequences produced by our methods and systems have a high IFR. We also determined how the sequences that do not fold back perform and whether their folded structure is very similar or different from the given structure. We also determined how much they may differ. First, we investigated this structural diversity by measuring the base pair distance of the folded structure to the given structure.

We used two microRNA let-7 family sequence structure pairs: MI0000416 from Drosophila, and MI0005057 from cattle. For both pairs, we sampled 50,000 sequences with distance filtration h, 1≤h≤20 and folded the sampled sequences. We computed the base pair distance of the folded structure to the natural structure. Distance 0 means the sequence folds back to given structure. We displayed the frequency of the sequences with respect to distance filtration and base pair distance, see FIGS. 14A and 14B. FIGS. 14A and 14B display the distribution of sequences with respect to sampling distance filtration and base pair distance between the folded structure and the natural structure. The x-axis denotes the relative base pair distance (normalized by the sequence length), the y-axis denotes the hamming distance, and the z axis denotes the frequency of sequence.

Note that a value of x=0 (the sequences that fold back to the given structure) corresponds to the IFR function of distance filtration. The IFR of MI0005057 drops much slower than the IFR of MI0000416. Moving along the x-axis, in MI0000416 many structures lie within small base pair distances to the given structure, while in MI0005057 a sharp decrease in frequency is observed. This means that in MI0005057 very few sampled sequences fold to a structure with small distance to the given structure, but they are more spread out. This implies that the sampled sequences of MI0005057 that do not fold to the given structure tend to fold to a structure having relatively large base pair distance compared to MI0000416. Sampled sequences in MI0005057 either try to preserve the given structure, or fold to a very different structure.

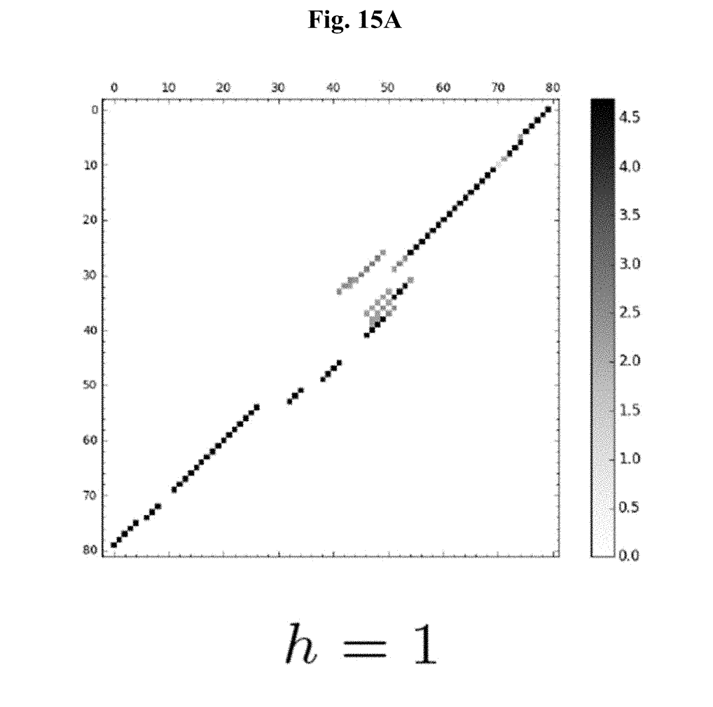

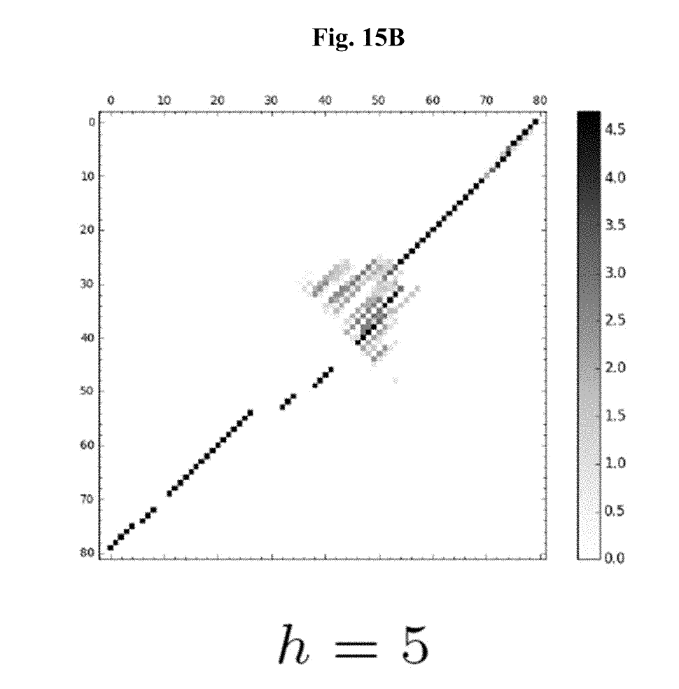

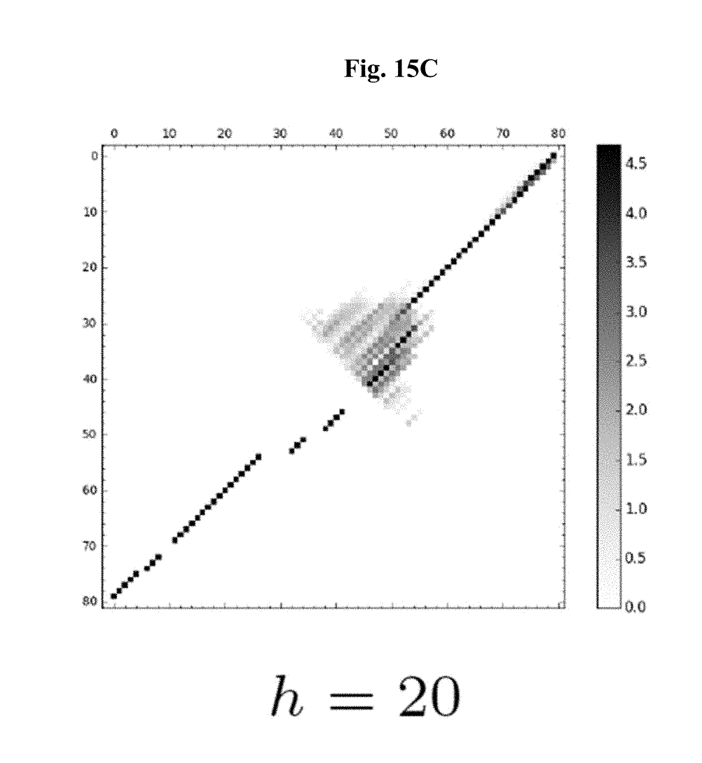

Next, we turned to the structural diversity at the base pair level. We plotted the base pairing probability (frequency) matrix of MI0005057 with distance filtrations of 1, 5 and 20, respectively, see FIGS. 15A-C. FIGS. 15A-C show the base pairing frequency matrix of MI0005057 with distance filtration 1 (FIG. 15A), 5 (FIG. 15B) and 20 (FIG. 15C). The upper right half of each graph corresponds to the base paring frequency, while the lower left part corresponds to the reference structure.

From the data of FIGS. 15A-C, it is clear that there is more diversity in the folded structure when distance filtration increases. This can be observed from the increasing ‘fuzziness’ in the base pairing probability matrix. Furthermore, the fuzziness is mainly observed at the loop region and a little bit at the beginning of the stem loop region.

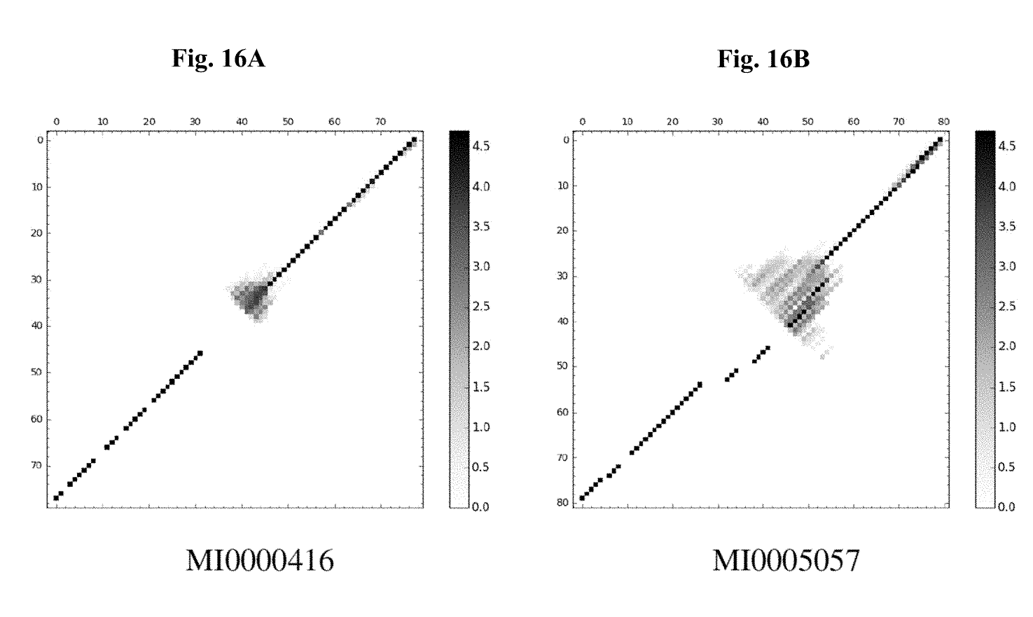

We then compared the base paring frequency matrix between MI0005057 (from cattle) and MI0000416 (from drosophila) with distance filtration 20, see FIGS. 16A and 16B. FIGS. 16A and 16B show the base pairing frequency matrix of MI0000416 (left) and MI0005057 (right) with distance filtration 20. The upper right half of each graph corresponds to the base paring frequency, while the lower left part corresponds to the reference structure.

MI0005057 is ‘fuzzier’ than MI0000416. This indicates that the sequences in MI0005057 have more structural diversity than those in MI0000416. It is believed that this is an identification of sequence structure pairs in high level organisms.

Neutral Path.

As discussed above, connectivity is an important property of neutral network. Thus, it is natural to ask whether there is a path connecting two given sequences on the neutral network of a structure. If so, how should one construct such a path? The neutral path problem has been studied in the context of random graph model (Göbel and Forst, 2002). However, according to what is known in the art, there is no efficient solution to this problem on a neutral network induced by a real folding method when the distance between the sequences is large. Our sampling method and systems describe herein give rise to an efficient heuristic to solve the neutral path problem. First, let us formulate the neutral path problem:

Given two sequences σ₁ and σ₂, that both fold to the same structure S, find a sequence of sequences (path) σ₁=τ₀, τ₁, . . . , τₖ=σ₂, such that for all τᵢ, 0≤i≤k, folds to S, where τᵢ₊₁ can be obtained by either a compatible point mutation of a nucleotide, or a pair mutation where the two nucleotides form a base pair, and k is the length of the neutral path.

Note that given two structures σ₁ and σ₂, that both fold to the same structure S, one can always construct a path between them using the methods and systems described above. Such a path is called an S-compatible path. Furthermore, there exists a minimum number of moves to move from σ₁ to σ₂. Such a number is called d_{S}(σ₁,σ₂), the S-compatible distance between σ₁ and σ₂. Clearly, we have ½d≤d_{S}≤d, for any S-compatible sequences. A neutral path with length equal to the S-compatible distance is called a shortest neutral path. For our neutral path problem, however, we do not require the path to be the shortest.

One naive approach to construct a neutral path between σ₁ and σ₂ is to exhaustively fold all shortest compatible paths between them. This is feasible when d_{S} is small, and with high success rate. However, when d_{S} increases, the number of shortest paths will explode (d_{S}!). Furthermore, a neutral path might still exist even when all shortest compatible paths are not neutral, because such a path might take a ‘detour’ to remain neutral.

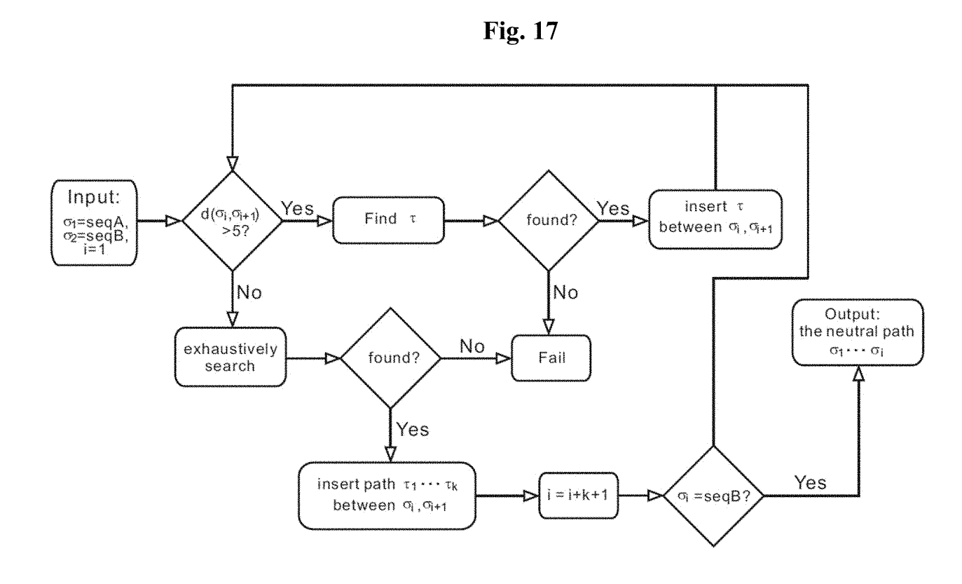

In our previous result, we observed that even at a large hamming distance, the sampled sequences have a high inverse fold rate, given the reference sequence actually folds into the reference structure. As a result, we developed a ‘divide and conquer’ strategy to solve the neutral path problem. We can utilize our sampling systems and methods described herein to set up an intermediate ‘anchor point.’ We can continue repeating this process until the distance is small enough to perform a brutal search, see FIG. 17. FIG. 17 is a flowchart of a method or system for finding a neutral path between a pair of sequences.

Scenario 1: d(σ₁,σ₂)≤5. In this scenario, we exhaustively searched for all shortest S-compatible paths between σ₁ and σ₂ and checked if the path was neutral by folding. Note that we always had d_{S}≤d, thus, in the worst case, we needed to check 5!=120 different paths and fold 2⁵=32 different sequences. This can be done very efficiently when the sequence length is shorter that 1000 nucleotides by the secondary structure folding method (Zuker and Stiegler, 1981; Hofacker et al., 1994). Indeed, it is possible that the shortest possible neutral path has length strictly greater than the S-compatible distance. However, we wanted to show that this case is rare. In order to validate this, we collected sequence structure pairs from microRNA let-7 family members across species (human, cattle lizard and other low level organism; 12 pairs for each class) as one endpoint of the path. Then for each sequence structure pair, we found inverse fold solutions by dual sampling 10,000 sequences with hamming distance 5, and collected those that fold back to the structure (on average 5,000 sequences are found) as the other endpoint. By exhaustively searching, we observed that for all of the sequence pairs, there exists a neutral path whose length is equal to the S-compatible distance. This indicates that exhaustively neutral path searching for sequence pairs with short hamming distance is efficient and feasible.

Scenario 2: d(σ₁,σ₂)>5. For the case of sequences having a large hamming distance, exhaustively searching is not as feasible because the number of sequences needed to be folded grows exponentially. Therefore, we used our sampler method to find an intermediate sequence τ_{S} to fold to the given structure such that d(σᵢ,τ)<d(σ₁,σ₂). Due to the high IFR of the dual sampler, such solution was not difficult to find.

Suppose σ₁ and σ₂ have hamming distance h. We sampled m sequences from σ₁ with respect to S with distance filtration h/2. Here we set m=1000. Due to the high IFR of the sampler, it is very likely to find sequences from the sampled set that fold to S (otherwise we increase m). We picked the one with minimum hamming distance to σ₂, denoted by τ_{S}. We have d(σ₁,τ_{S})=h/2=h₁ and d(τ_{S},σ₂)=h₂, where h₁+h₂≥h. If h₂>h we find the process fails and we conclude we cannot find a neutral path between σ₁ and σ₂. Otherwise, we repeat the searching process between σ₁ and τ_{S}, and between τ_{S} and σ₂. If h₁(h₂)≤5 then take the first scenario (otherwise take the second scenario). If the searching does not fail, the process ends up with a neutral path because both h₁ and h₂ are smaller than h in the recursion.

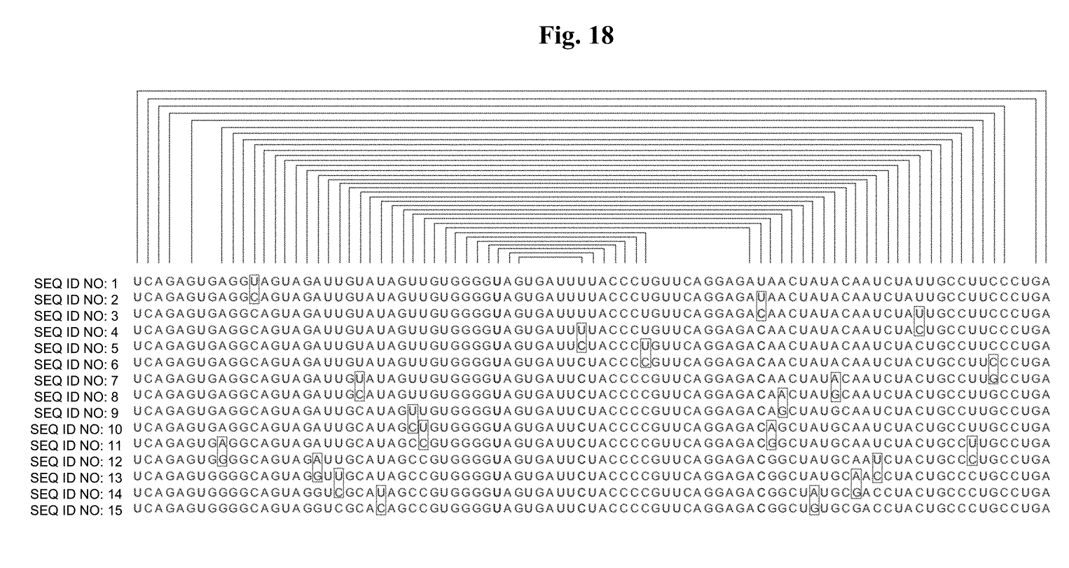

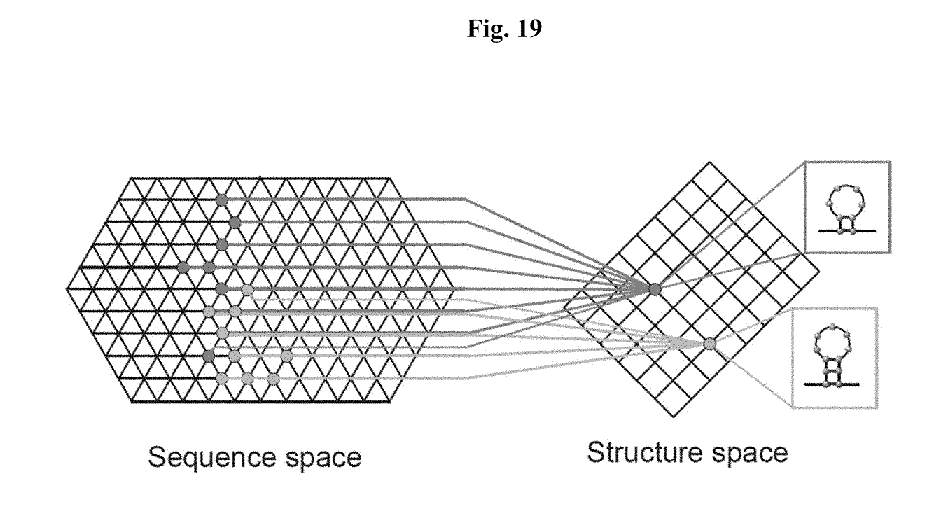

We show an example of a neutral path found by our system and methods in FIG. 18. FIG. 18 shows a neutral path found by the systems and methods disclosed herein from the natural sequence of hsa0000063 to a sequence having a hamming distance of 20 that folds to the structure of hsa0000063. The path has 14 steps, 8 point mutants and 6 base pair mutants.

Success rate of the method: For human microRNA MI0000063 we first used the natural sequence and structure pair and sampled 100 sequences with distance filtration 20. In these 100 sequences we found 19 fold that back to the given structure. We paired each of them with the natural sequence and computed a neutral path, which resulted in 18 successes and 1 failure.

For human microRNA MI0000067 we did the same experiment for distances 20 and 40. For distance 20 we found that 85 out of 100 sequences fold back to the given structure, with 83 successes and 2 failures. For distance 40 we found that 53 out of 100 sequences fold back to the given structure, with 49 successes and 4 failures.

For a low level organism microRNA, MI0026171, which is a Branchiostoma micro RNA, at distance 20 we found 22 out of 100 sequences that fold back, with 16 successes 6 failures.

Thus, the methods and systems described herein have a good success rate of finding a neutral path, even at large hamming distance. In addition, we observed that higher level organisms seem to have a better success rate. Without being bound to a particular theory, this may due to higher robustness/IFR in higher organisms.

### Discussion

DP routine. The method described in Levin et al., 2012 is a constrained version of the method described in Waldispuhl et al., 2008, thus, the time complexity when k=n is O(n⁵). One of the many differences between the method described in Levin et al., 2012 and the method and systems described herein is the DP routine. To facilitate DP, a hierarchical structure over the subproblems is necessary. In Waldispuhl et al., 2008, the sub-problems are given by all the sub intervals of [1,n], and the hierarchical structure is given by concatenation. In Levin et al., 2012, the contribution of corresponding sub-problems will be set to 0 when encountering a conflicting substructure but that would not affect the time complexity, hence the computational complexity is still O(h²n³). One of the problems with that approach is that the given structure has not been best utilized. Whereas in certain methods and systems described herein, the hierarchical structure is given by loops under nesting relations, which is essentially a tree (the parent problem of any sub-problem is unique). This allows for computation of the partition function from inside to outside (bottom to top from the tree prospective). Because the routine is purely given by the structure, no redundant information is computed, in contrast to the method of Levin et al., 2012. As a result, the methods disclosed and described herein lead to a much more powerful and efficient approach.

### Example 3

In this section we report various properties of drift walks according to the methods and systems described herein. We organized these by Hamming distance and base-pair H-distance. We also studied switches and analyzed how these walks navigate through sequence space-passing through the interior and boundaries of neutral networks. In addition, we integrated and discuss our results and put them into context with observations in evolutionary optimization.

Multiplicity of RNA MFE-Structures.

As discussed, sequence degeneracy, i.e., the existence of multiple MFE-structures is frequently observed for RNA sequences. This phenomenon is generic, holding for natural sequences (see FIGS. 20A and 20B), and random sequences alike.

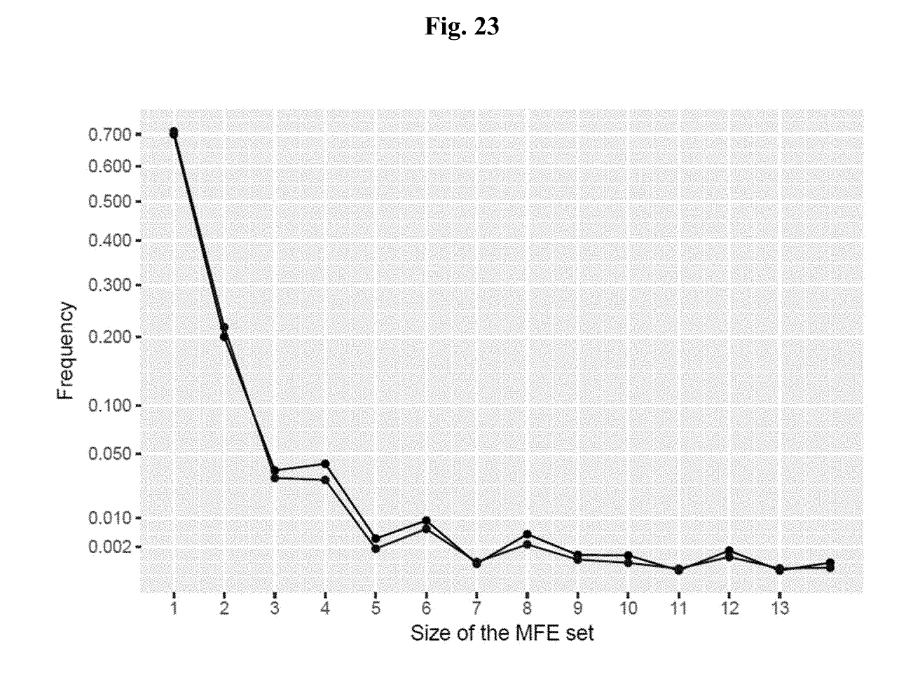

We began by taking a closer look at degeneracy, displaying the distribution of the number of MFE structures for random sequences of length 100, as well as miRNAs of length less than or equal to 100 in FIG. 23. FIG. 23 shows the frequency distribution of the sizes of the MFE sets: for random sequences of length 100 (blue) and precursor miRNAs of length at most 100 (black). Ten million sequences of the former, and about eighteen thousand sequences of the latter type (from miRBase (Kozomara and Sam Griffiths-Jones, 2014), were considered. We employed the RNAlib library from the Vienna RNA package (Hofacker, 2003); however, instead of computing MFE structures directly, we used the suboptimal structure generator with the energy threshold set to zero. From this data we concluded that about 29.5% of sequences of length 100 are degenerate and the majority of such sequences have exactly two MFE-structures. We referred to a pair of structures in the MFE-set of a degenerate sequence (regardless of the size of the set) as an MFE-pair.

We observed that sequence-degeneracy is conserved amongst the neighbors of a (random) degenerate sequence. We called a sequence having exactly k distinct MFE structures, k-degenerate. Note that a 1 degenerate sequence has exactly one MFE structure and is thus nondegenerate. Studying a sample of 10⁵ randomly chosen sequences, we observed that on average 87% of the neighbors of a 1-degenerate sequence are 1-degenerate, while for 2- and 3-degenerate sequences this percentage is 59% and 47%, respectively.

FIG. 24 shows the distribution of base-pair distance of MFE pairs obtained from a sample of 10⁶ random degenerate RNA secondary structures. The mean base-pair distance is 6.7 and the maximum distance observed is 72. About 80% of the MFE-pairs differ in less than or equal to 10 base-pairs.

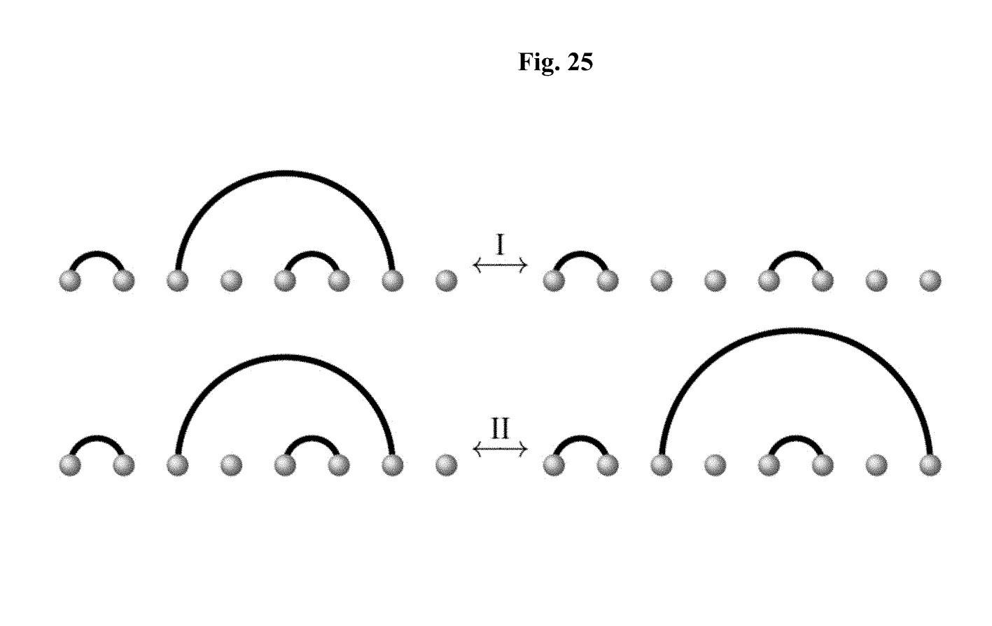

In view of the fact that MFE structures of degenerate sequences are typically similar in terms of their base-pair distances, we consider the following two moves (see FIG. 25):
I: the addition or deletion of a single base-pair;
II: suppose (i, j) is a base-pair and k>0, such that (a) the bases in [i−k,i] or (b) [j,j+k] are unpaired: the shift mapping (i,j), to either (a): (i−k,j) or (b): (i,j+k).

It is straightforward to verify that successive applications of the above connects any two structures. For MFE-pairs we observe:
46.2% are related by a single type II move, where the length of the shift is either 1 or 2;
13.7% of the pairs are related by a single type I move, i.e., by the addition of a single base-pair (of arbitrary length);
10.1% are related by adding a stack of two or more parallel arcs, possibly involving wobble base-pairs,
5% are related by removing a set of arcs and adding another set (not of type II),
23.9% are related by shifting more than one arc.

In FIG. 25, secondary structure moves of type I and II are displayed: type I moves add or delete a base pair, while type II moves shift a base pair on either side without sliding over base pairs.

The majority of MFE-pairs are related by type II moves, which arguably stems from the fact that, as opposed to type I moves, these moves do not alter the loop decomposition of the structure. Thus, as free energy is computed via loops within the structure, the energies of two, type II related structures are very similar.

After inspecting specific properties of MFE-pairs, S, S′, we studied under which conditions there exists a sequence T, such that S, S are a MFE-pair of T. In other words, is being related by the above moves sufficient for S, S′ to be an MFE-pair? We called such a sequence, τ, a common inverse fold of S,S′.

Suppose, σ, is a sequence that folds into S and that a is, in addition, compatible with S′. By the intersection theorem (Reidys et al., 1997) there always exist sequences that are compatible with both S and S′, although it is not guaranteed that any one of them realizes S as an MFE-structure, however, in praxis, if the base-pair distance between S and S′ is small, we were able to almost always identify such a sequence. To construct a common inverse fold, we considered the distance between a and the neutral network of S′:

d(σ,S′)=E(S′,σ)−E(S,σ)≥0,

By construction, this distance is zero if, and only if, S′ is contained in the MFE-set of σ, i.e., σ is in the intersection of the iNeutral networks of S and S′.

To find a common inverse fold for S, S′, we recursively constructed a walk, which starts at σ₀=σ and which, at each step, i identifies a neutral neighbor with regard to S of σᵢ, that is compatible with S′ and whose distance to the neutral network of S′ is less or equal than that of σᵢ. Preference was given to such sequences, whose distance is minimal. In case of either a sequence, σ_{N} of distance zero, or after a certain number (10⁴ in our run) of steps have been undertaken, the method stops. By construction, such a σ_{N} realizes both S and S′ as MFE-structures. In case the method cannot locate such a σ_{N}, a new walk is initiated (up to ten trials), originating from a different sequence σ, obtained by inverse folding S and that is compatible with S′.

We tested this method for pairs S, S′ that were (a) MFE-structures of degenerate sequences and (b) random pairs but connected by a move of type I or II. The latter pairs were obtained by first choosing a random MFE structure and then randomly removing or shifting a base-pair in it, to obtain a new structure. Executing the method for 100 pairs in each group, respectively, we were able to construct a degenerate sequence having S, S as MFE-pair for all but two pairs of group (a) but only for five out of the 46 for group (b) pairs. So, the method almost always identifies a common inverse fold for the pairs of group (a) but seldom succeeds for pairs of group (b). Accordingly, a pair of secondary structures related by a single type I or type II move cannot be expected to have a common inverse fold in general and so the above moves are not enough to characterize MFE pairs.

Drift Walks.

Schuster et al., 1994, studied the distribution of the maximal Hamming distances that neutral walks can reach, a random variable closely connected to the diameter of the underlying neutral networks. Their computational analysis revealed that 21.7% of the walks traversed the entire sequence space, achieving the maximum possible Hamming distance from the origin.

In this section we extend this type of analysis to iNeutral or drift walks, which add an entirely new dimension as they alter not only sequence, but also structure. Let us call a walk in sequence space originating at σ₀ monotone if and only if it never decreases the Hamming distance of the sequences constructed and free if no restriction on Hamming distance is being imposed. In certain embodiments drift walks are free unless declared.

The following analysis of drift walks is based on the base-pair H-distance, which is the Hausdorff-distance between sets of structures. To construct the Hausdorff distance, we assumed the base-pair distance, d, on RNA structures are fixed, however the following procedure can extend any secondary structure distance to a distance between sets of structures.

First we defined the distance between a structure s and a set of structures B as the minimum of the distances between s and the elements of B i.e. D(s,B)=min_{s′∈B}d(s,s′). As a result, D(s,B)=0 if and only if s∈B. Secondly, we extended this from a singelton {s} to a set of structures, A, as follows: ^{\vec{}}D(A,B)=max_{s∈A}D(s,B). Note that ^{\vec{}}D(A,B)=max_{s∈A}D(S,B) and ^{\vec{}} D(B,A)=max_{s′∈B}D(s′,A) are not necessarily equal, hence we symmetrize

D(A,B)=max{^{\vec{}}D(A,B),^{\vec{}}D(B,A)}.

It is apparent that D is indeed a metric (Rockafellar and Wets Roger, 1998), i.e., if D(A,B)=0, then A=B and we have the triangular inequality D(A,C)≤D(A,B)+D(B,C).

The base-pair H-distance, D, has the same range of values as d and the Hausdorff distance between two sets A, B is less or equal than the diameter of AuB and is greater than or equal to the minimum distance between an element of A and an element of B. In particular, we have

D({a},B)=^{\vec{}}D(B,{a})=max_{b∈B}d(a,b).

Equipped with this notion of distance between sets of RNA structures, we were able to proceed with our analysis of drift walks.

Maximal Hamming Distance.

In comparison to the monotone neutral walks studied in Schuster et al., 1994, we found that the mean maximal Hamming distance reached from the origin along free drift walks is 82.5, while the mean maximal base-pair H-distance to the MFE-set of the starting sequence is 60.3. This phenotypic reach is notably high, given that the maximal base-pair distance between structures of length 100 equals 100. Accordingly, a free drift walk travels typically deep into both sequence, as well as structure space.

Hamming Distances.

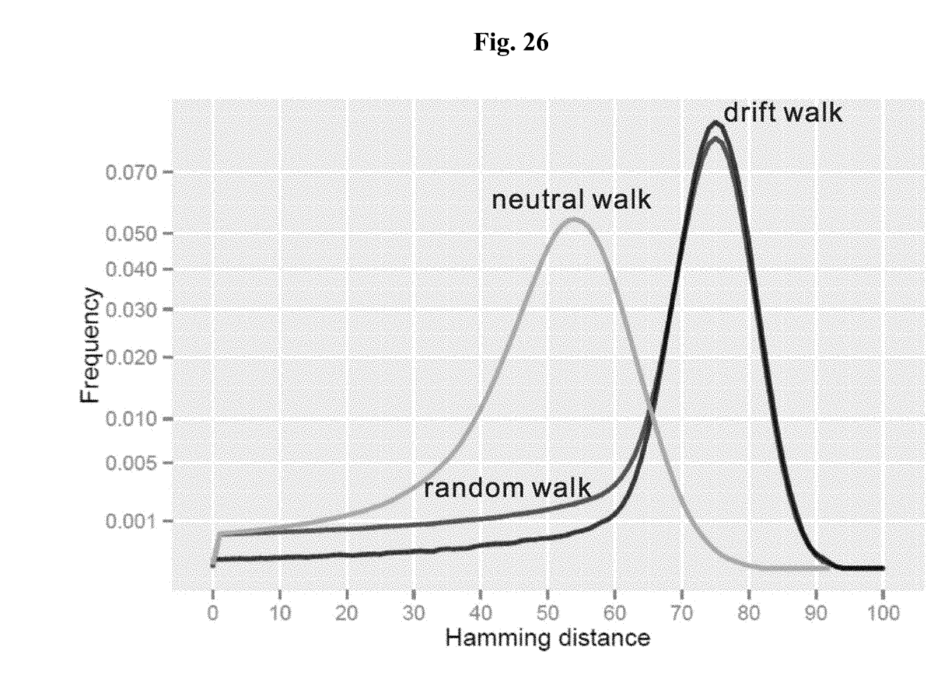

In FIG. 26 shows the distribution of Hamming distances along neutral, drift and random walks. The frequency distribution of Hamming distances to the starting sequence is shown along drift walks, random walks and neutral walks. Similar to random walks, drift walks spend the majority of their time around Hamming distance 75, the largest Hamming distance for 4-letter alphabets. Neutral walks, in contrast, are located around Hamming distance 54 and are thus much more constraint and correlated with the starting sequence. The figure shows that random walks and drift walks reside at a Hamming distance of 75, which is for 4-letter alphabets the largest Hamming distance in sequence space. This finding was expected for random walks because by construction their folded structures do not constrain the next step in the walk. The similarity of drift walks and random walks suggests that drift walks are moving freely in sequence space despite the fact that their next step is severely constraint by being only able to move to sequences whose MFE set has a non-empty intersection. In contrast, neutral walks reside around a Hamming distance of 54. This shows that neutral walks are far more constrained by the requirement of preserving the singelton MFE set and much more correlated with the starting point of the walks. Drift walks, despite being very different from random walks, as they can only change structure at degenerate sequences and in specific ways, are capable of percolating sequence space easily.

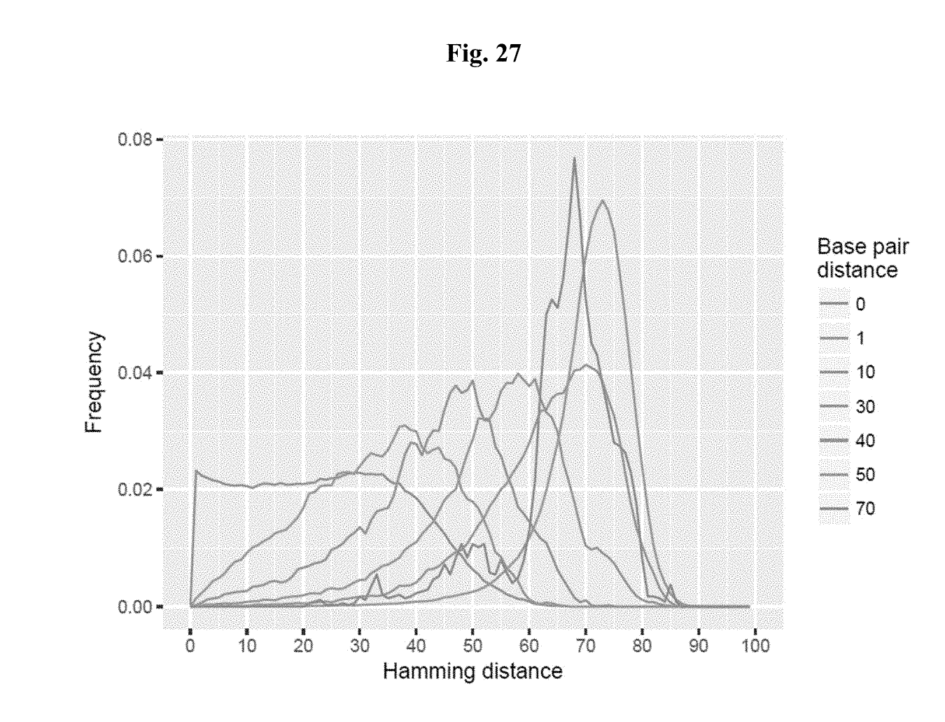

We next analyzed the probability of a free-drift walk to reside in Hamming distance d, conditional to a fixed base-pair H-distance. FIG. 27 displays these distributions as curves indexed by the respective base-pair H-distances. In case of base-pair H-distance zero, we only counted the contributions to a given Hamming distance in which we encountered sequences that have exactly the same set of MFE-structures as the origin-sequence. Note that along a drift walk, the MFE-set can change and then “revisit” the MFE-set of its starting sequence. All such revisits contribute to the Hamming distance zero curve in FIG. 27 where we see that the likelihood of encountering the original MFE-set along the walk decreases after Hamming distance 30 and becomes practically zero for Hamming distances greater than 60. Furthermore, FIG. 27 indicates that larger base-pair H-distances correlate with larger Hamming distances. This supports the hypothesis that free drift walks are continuous: small changes in Hamming distances produce small changes in base-pair H-distance but accumulate over the length of the walk, leading to novel, different RNA structures. The figure allows us to observe that as the structure H-distance increases, the distribution of Hamming distances converges to a skewed normal, localized around Hamming distance 72. This indicates that when reaching a large structure H-distance to the origin, a free drift walk effectively behaves like a random walk, losing any memory of the MFE-set of its origin.

Base-Pair H-Distances.

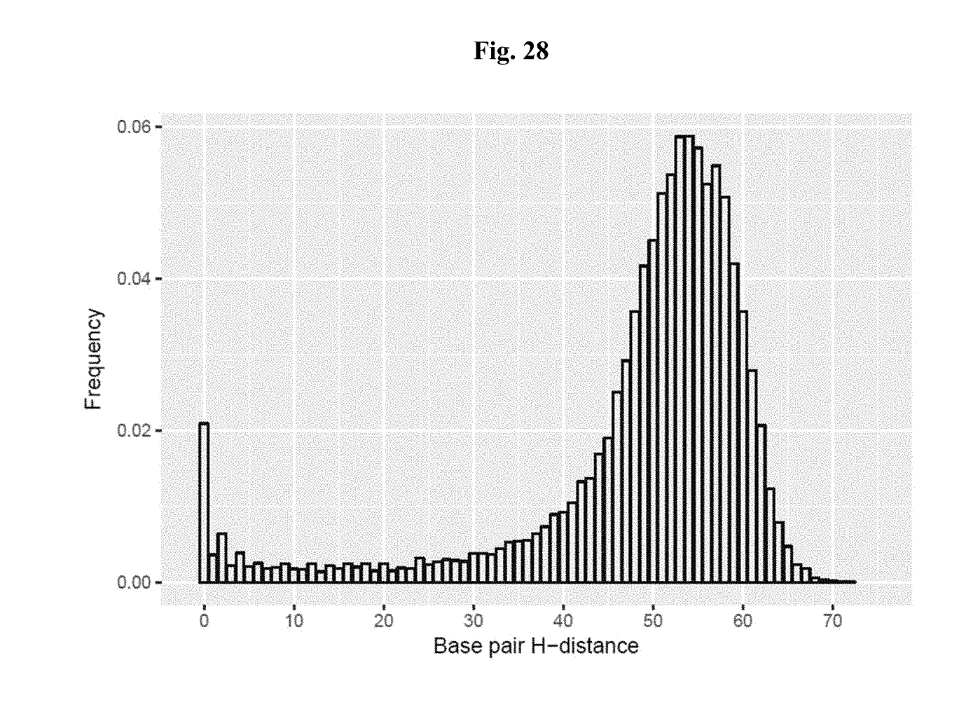

We next shifted our focus from Hamming distances to base-pair H-distances, i.e., how free drift walks move in structure space. FIG. 28 shows the frequency distribution of base-pair H-distances with respect to the MFE set of the origin of the walk. In FIG. 28, the distribution of base-pair H-distances to the MFE set of the starting sequence along free drift walks of length 2000 is shown. Each bar represents the frequency of the steps at which the MFE set of the current sequence has a fixed base-pair H-distance. The mode of the distribution is located at 54, which is close to the expected value of the base-pair distance between two MFE structures of random sequences. The figure displays how many steps the free drift walk resides in certain base-pair H-distances and we observed that free drift walks spend a significant amount of time (42 steps on average) in base-pair H-distance zero, i.e., having the same MFE-set as the origin. The figure shows that the drift walks spend the majority of the time between base-pair H-distances 45 and 65. The distribution is localized at distance 54, which is almost identical to the expected value of base-pair distance between two MFE structures (the latter equals 53.7 based on 10⁶ pairs of MFE structures of random sequences). The drift walk resides rarely in between base-pair H-distances 1 and 44, which can be understood by the close correlation between Hamming and base-pair H-distance and the fact that a drift walk moves away from the origin into the largest Hamming distance class.

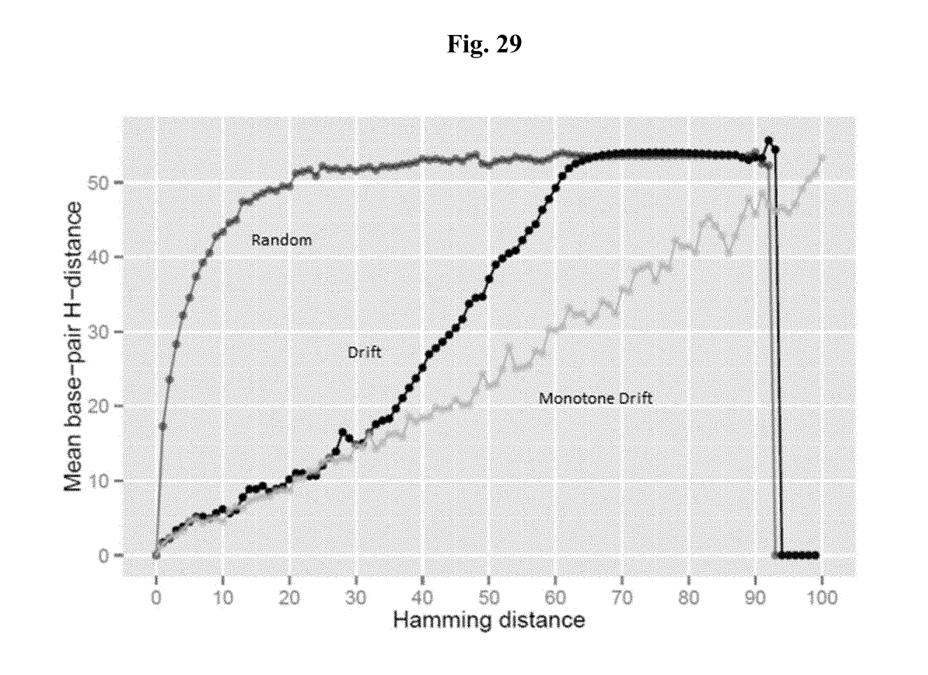

In FIG. 29 we display the mean base-pair H-distance as a function of Hamming distance for free drift walks, monotone drift walks, as well as random walks. The average base-pair H-distance to the MFE set of the starting sequence, σ, over Hamming distances assumed by a drift walk, random walk and monotone drift walk of 30000 steps. Drift and random walks exhibit a similar behavior at Hamming distances larger than 70. The mean base-pair H-distances of monotone drift walk reflects the gradual structural variation as a function of Hamming distance. The underlying data contain instances where drift walks move to Hamming distance 40 without changing their MFE-sets, while others achieve a base-pair H-distance of 48 with just one mutation. FIG. 11, however, shows that on average the base-pair distance grows almost linearly with the Hamming distance, for up to Hamming distance 70 and then stabilizes. We can furthermore conclude that, compared to random walks, free as well as monotone drift walks need to travel to significantly larger Hamming distances from the original sequence to reach the same base-pair H-distance. This observation confirms the finding that drift walks change the underlying structure in a continuous fashion. By construction, monotone drift walks move quickly into the maximal Hamming distance 100 and realize a mean base-pair H-distance comparable to the maximal base-pair H distance obtained in random and free drift walks.

Interior and Boundary of Neutral Networks.

By construction, a drift walk travels within, as well as in between, neutral networks. This suggests the following dual perspective on sequence degeneracy: a sequence, σ, is either contained in the neutral network N_{S} of a unique structure S, in which case the sequence is non-degenerate, or otherwise it is contained in multiple neutral networks, N_{S},N_{S}, . . . , i.e., it is degenerate. In the former case, σ is referred to be in the interior of N_{S} and in the latter a is in the boundary of N_{S}, N_{S′}, . . . .

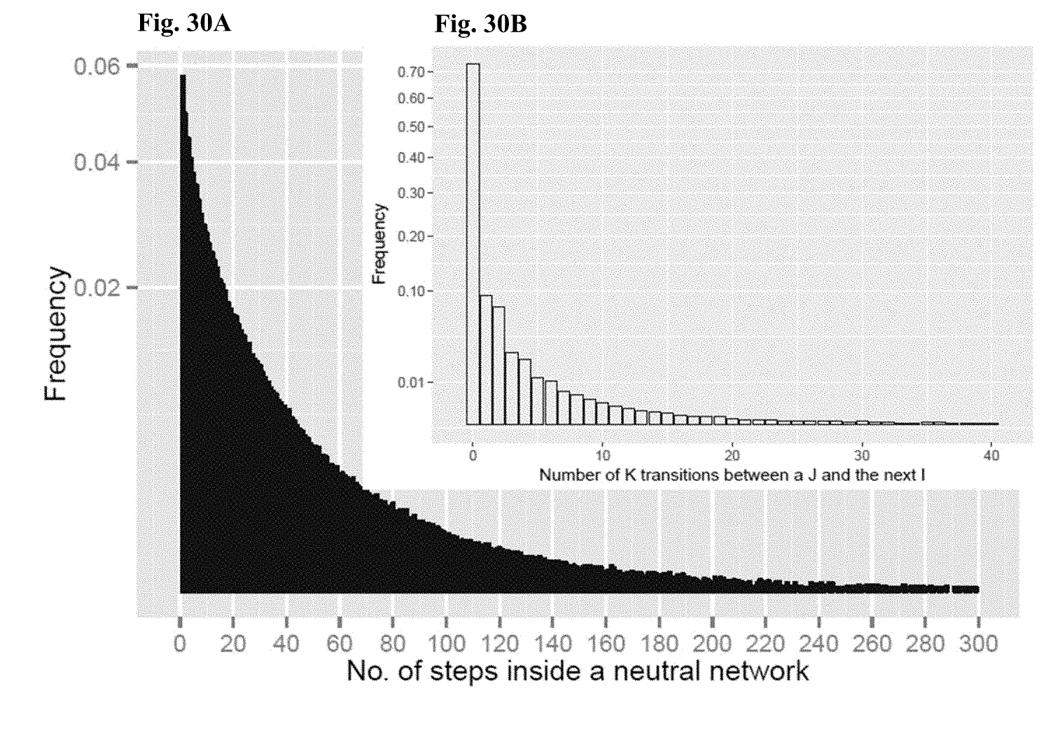

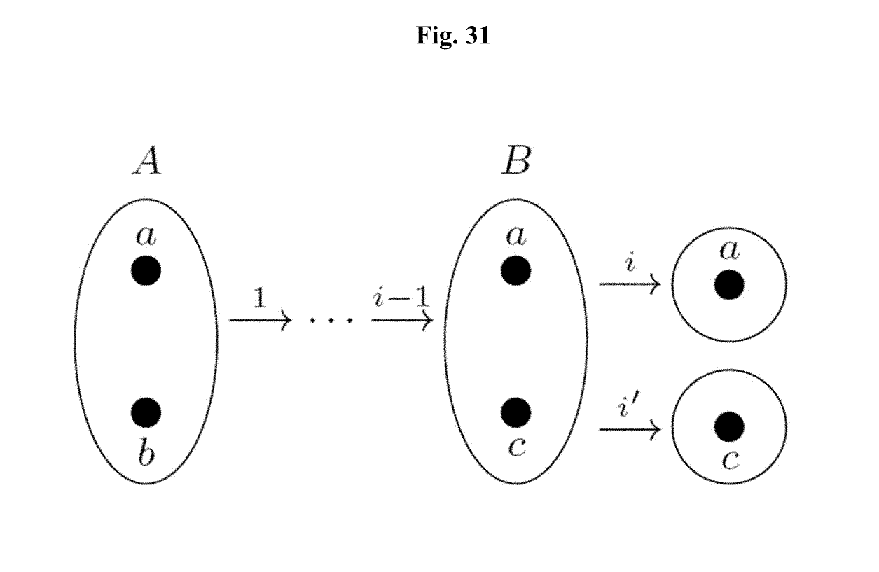

A I-switch in a drift walk is a transition from a degenerate sequence to a nondegenerate sequence, thus moving from the boundary to the interior of a neutral network N_{S}. A J-switch in a drift walk is a transition from a non-degenerate sequence to a degenerate sequence, thus moving from the interior to the boundary of N_{S}. By construction, in between these switches, the drift walk resides in the interior of N_{S}. FIGS. 30A and 30B display the distribution of the number of steps a drift walk spends in the interior of a neutral network. The frequency distribution of the times (number of consecutive steps) spent in the interior of neutral networks is shown. The insert (FIG. 30B) shows the frequency of numbers of K-switches contained in between J-, I-switches. The figure shows that in the majority of cases there is no K-switch between a J- and I-switch. On average, a drift walk resides for 24 steps with a standard deviation of 26.5 in the interior of a neutral network and 10 steps with a standard deviation of 18 in the boundary. Also, on average, 81.4% of the iNeutral neighbors of a sequence contained in the boundary of neutral networks have an identical MFE-set, i.e., they are located in the boundary of exactly the same neutral nets. For sequences in the interior of a neutral network, 95.6% of their iNeutral neighbors remain in the interior.

By construction, at I- and J-switches, the MFE sets of the underlying sequences change in cardinality. However, there are more ways in which MFE sets can evolve along drift walks. To this end we considered pairs of successive sequences, (σᵢ₋₁,σᵢ) and called the step producing σᵢ a switch if, and only if, the MFE-set of σᵢ₋₁ and σᵢ are different and a neutral step otherwise. In free drift walks, about 92% of the steps are in fact neutral and 8% are switches. Thus:
1. Type I. a switch from the boundary of a neutral network to its interior;
2. Type J. a switch from the interior of a neutral network to its boundary;
3. Type K. a switch within the boundary of neutral networks.

In I-switches, σᵢ is degenerate and σᵢ₊₁ is non-degenerate and vice versa for J-switches. At K-switches both: σᵢ, σᵢ₊₁ are degenerate. A drift walk decomposes into consecutive pairs of I- and J-switches with K-switches in between consecutive J-, I-switch pairs, see FIGS. 14A and 14B. We can report that 71% of the switches in drift walks are I-, J-switches while 29% are K-switches.

As mentioned before, between a consecutive pair of I- and J-switches, the drift walk resides in the interior of a neutral network, i.e., the walk is neutral. However, in between consecutive J- and I-switches, the walk resides in the boundary, and either preserves the MFE-set or performs K-switches. A closer look at the number of K-switches contained in between J- and I-switches of a drift-walk is provided in the insert of FIG. 30. The average number of such K-switches is 0.8.

A drift walk starting in the interior of a neutral network requires an average Hamming distance of 15.6 in order to reach the boundary, at which point we have a J-switch. In contrast, starting with a sequence, contained in the boundary, a drift walk requires an average Hamming distance of 7.1 in order to reach a either a I- or K-switch.

This sheds some light on as to why in FIG. 28 we observe a much higher frequency to remain in base-pair H-distance zero than in distance 1 and 2. Namely, 70% of the drift walks originate from the interior of a neutral network while 30% originate from the boundary. For the base-pair H-distance to increase the former walks have to reach the boundary first and until the boundary is reached the base-pair H-distance remains zero.

We then turned to the difference between the base-pair H-distances before and after a switch as its contribution to the distance. The mean contribution of the switches of drift walks of length 2000, distinguishing I-, J- and K-switches are −101, 140 and 14 respectively. The sum of the contributions of the three types, for degenerate as well as non-degenerate starting sequences, amounts to a mean base-pair H-distance of 53, which is very close to the mean base-pair distance between two single MFE structures.

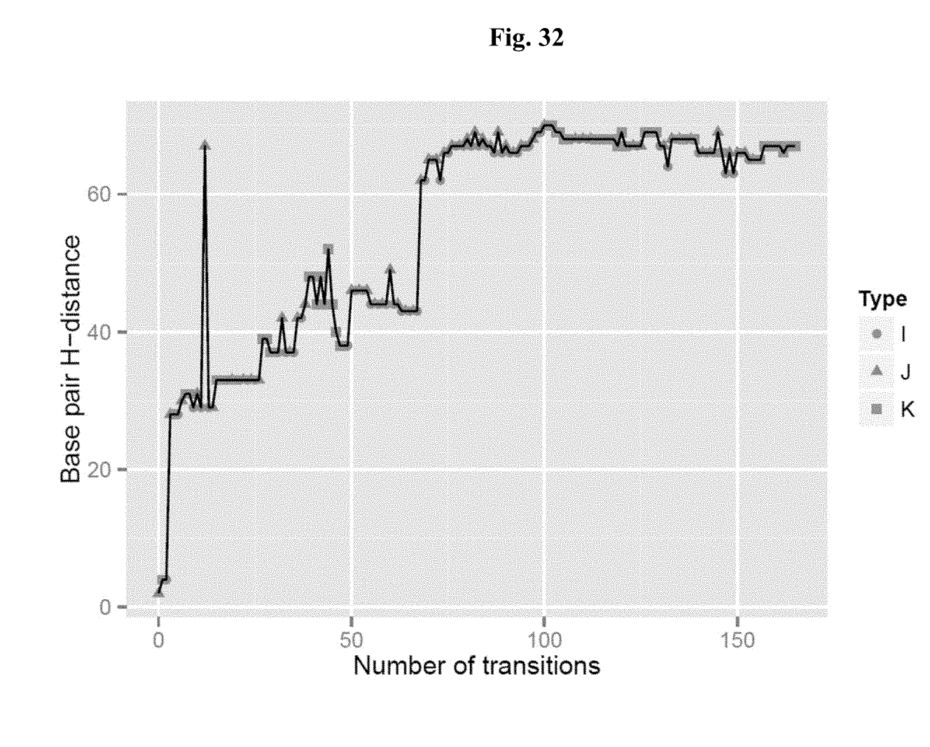

FIG. 32 depicts the evolution of the base-pair H-distance along a particular, free drift walk having 2000 steps. The evolution of the base-pair H-distance along a drift walk: the walk consists of 2000 steps, originates in the boundary, and we label the specific types of switches. J-switches predominantly contribute positively to base-pair H-distance, while I-switches contribute typically negatively. K-switches can affect the base-pair distance both ways, but their contribution is smaller than that of I- and J-switches. The large peak observed around step 20 corresponds to a J-switch in which a very dissimilar MFE structure is added to the MFE set. This event is immediately followed by an I-switch, in which this structure disappears and the walk returns to the interior of the neutral network.

Connecting Structures with Drift Walks.

The above analysis shows that drift walks connect very different MFE secondary structure structures. We then asked whether any two MFE structures, S, S′, can be connected via some drift walk. This would imply that any MFE RNA phenotype can be realized from any starting sequence by means of a drift walk—an ideal scenario for evolutionary optimization.

We provide strong evidence herein that indeed any two MFE structures can be connected by a drift walk. To this end, we devised a method that, given S, S′ obtains first the inverse folded sequences σ₀ for S and σ′₀ for S′. The method generates inductively two drift walks originating at σ₀,σ′₀, respectively. Suppose σᵢ₋₁,σ_{i-1′} are constructed; then σᵢ is chosen among the iNeutral neighbors of σᵢ₋₁ in such a way that its MFE set is closer or equal (in terms of base-pair H-distance) to that of σᵢ₋₁′. We proceeded analogously with σ_{i-1′}. In case no base-pair H-distance reduction could be obtained for 2000 steps, a 20 step free drift walk was performed, originating at, randomly, either σᵢ or σ_{i′}. The method was set to terminate if either four hundred thousand steps were taken or base-pair distance zero was achieved.

In certain embodiments, methods and systems described herein were able to connect all but 6 out of 51 pairs of random MFE structures.

A drift walk represents a slight generalization of a neutral walk, i.e., a walk in which the phenotype is entirely preserved. Previous folding methods of RNA secondary structures randomly select a MFE structure (Hofacker, 2003), because typically MFE sets are composed by very similar structures. Even in light of multiple counterexamples, for instance RNA Riboswitches and Leptomonas collosoma sIRNA, this stipulation typically holds.

We reported that around 30% of the sequences have multiple MFE structures, which suggests generalizing the notion of neutral neighbors and neutral walks to iNeutrality and Drift walks. Clearly, in absence of selective pressures, none of the MFE structures are given any preference and the question arises how a drift walk evolves its associated phenotypes. In contrast to neutral walks, that by construction never leave a certain neutral network, the framework of drift walks allows distinguishing when such walks reside in the interior and the boundary of a neutral network, respectively.

Our analysis of Hamming distances, and in particular of mean Hamming distance, shows that, in contrast to neutral walks, drift walks, after sufficiently many steps, do not retain any memory of the origin. Neutral walks reside around a mean Hamming distance of 54, which shows that they are much more constraint and drift walks behave very similar to random walks residing mostly in Hamming distance 75, which is the largest Hamming class for sequence lengths of 100.

As for base-pair H-distances, again drift walks behave as random walks, the only difference being the rate at which the base-pair H-distance increases over the Hamming distance of the walk. Random walks almost immediately lose any correlation with the MFE set of the origin, while drift walks gradually achieve structural change. Despite the fact that MFE sets of fixed sequences contain typically very similar structures, the multiplicity of phenotypes facilitates cumulatively significant, structural change.

One of the most striking questions is whether the entire structure space is connected by drift walks. This question should be considered in the context of the evolutionary optimization, where typically some population of RNA sequences evolves by means of a stochastic relocation-deletion scheme: a flow-reactor or birth-death process of some kind. A typical scenario here is the following: starting from a population of sequences, all of which realize a particular structure, we try to find a given target structure. The evolution of the population then is driven by granting selective fitness advantages to sequences, when they fold into structures that are close to the target. Because these selective advantages are based on the phenotype, there exist sequences in the population folding into them, however, it is not clear how and to what extend phenotypes change in the course of the optimization. In a computational study (Fontana and Schuster, 1998), Fontana and Schuster identified evolutionary relays in their optimization experiments, in which two subsequent structures are connected by specific moves.

Methods and systems described herein connect two structures S, S′ by a drift walk by identifying an iNeutral path starting at S and ending at S′. In certain aspects, this is tantamount to constructing a path to the target structure via iNeutral neighbors, i.e., at each transition the sets of MFE structure have nonempty intersections. The methods and systems, thus, construct a drift walk under the selective pressure of minimizing structural distance and with a priori gradual structural changes as the MFE sets of fixed sequences are very similar. The point here is, that, if drift walks are able to connect any two structures, then there exists an iNeutral path, along which structural changes are by construction gradual. As a result, this provides deeper insight in the evolutionary relays described above.

This means that locally, i.e., each step, the drift walk realizes phenotypes that have optimal biophysical stability and by maintaining this feature, any phenotype can be connected to by such a walk. The maintenance of a locally optimal free energy, in certain aspects tantamount to stability of intermediate phenotypes, does not constrain into what phenotype can be derived. This enhances our understanding as to what extent transitions between phenotypes are driven by selective advantages. Drift walks show that new phenotypes can be realized via chains of iNeutral neighbors.

In certain aspects, our analysis described herein shows that considering a set of RNA structures instead of restricting to a single MFE structure has relevant implications. The mere existence of multiple phenotypes leads to a new class of walks in which phenotypes evolve in very specific ways, yet along which massive phenotypic changes can be achieved. This calls for the systematic inclusion of the partition function into this framework. This observation also unearths intersections between neutral networks that have not been considered before. This enables us to organize the space of RNA phenotypes into a network whose edges are given by the intersections between neutral networks.

The described techniques can be implemented on computers. The computers include at least a processor, memory, and storage for storing and executing programs, and for storing information needed by the program or generated by the program. In some embodiments, each computer may include additional hardware such as a graphics processing unit (GPU), a field-programmable gate arrays (FPGA), or other specialized processors that accelerate certain operations. These specialized processors may be used in conjunction with general-purpose processors to accelerate the execution of the described techniques. The computers may be connected to other computers using a network in a manner that allows for the parallel execution of multiple tasks so that the execution of the techniques is accelerated. The aspects of the computers executing the techniques may also be distributed among multiple computers to facilitate parallel processing. In one embodiment of a computer system executing the described techniques, computers with GPUs may cooperate with other computers with FPGAs and/or other computers without specialized processors to execute the described techniques in parallel. In another embodiment of a computer system executing the described techniques, supercomputers may be employed to execute certain aspects of the techniques in a more accelerated manner. In still further embodiments of a computer system executing the described techniques, a variety of different computers and supercomputers are employed to address different aspects of the described techniques. A variety of other configurations of computing resources may be employed to provide the desired accelerated execution of the techniques disclosed herein.

It will be appreciated by those skilled in the art that the present disclosure can be embodied in other specific forms without departing from the spirit or essential characteristics thereof. The presently disclosed embodiments are therefore considered in all respects to be illustrative and not restricted.

All publications, patents and patent applications cited in this specification are incorporated herein by reference in their entireties as if each individual publication, patent or patent application were specifically and individually indicated to be incorporated by reference. While the foregoing has been described in terms of various embodiments, the skilled artisan will appreciate that various modifications, substitutions, omissions, and changes may be made without departing from the spirit thereof.

### REFERENCES

Barrett, C., Huang, F., and Reidys, C. M. (2017). Sequence-structure relations of biopolymers. Bioinformatics, 33(3), 382-389.
Bernhart, S., Tafer, H., Mückstein, U., Flamm, C., Stadler, P., and Hofacker, I. (2006). Partition function and base pairing probabilities of RNA heterodimers. Algorithms Mol. Biol., 1, 3.
Busch, A. and Backofen, R. (2006). INFO-RNA—a fast approach to inverse rna folding. Bioinformatics, 22(15), 1823-31.
Ding, Y. and Lawrence, C. E. (2003). A statistical sampling algorithm for RNA secondary structure prediction. Nucleic Acids Res., 31, 7280-7301.
Garcia-Martin, J. A., Bayegan, A. H., Dotu, I., and Clote, P. (2016). RNAdualPF:software to compute the dual partition function with sample applications in molecular evolution theory. BMC Bioinformatics, 17(1), 424.
Göbel, U. (2000). Networks of minimum free energy RNA secondary structures. PhD Thesis, University of Vienna.
Grüner, R., Giegerich, R., Strothmann, D., Reidys, C. M., Weber, J., Hofacker, I. L., Stadler, P. F., and Schuster, P. (1996a). Analysis of RNA sequence structure maps by exhaustive enumeration I. structures of neutral networks and shape space covering. Chem. Mon., 127, 355-374.
Grüner, R., Giegerich, R., Strothmann, D., Reidys, C. M., Weber, J., Hofacker, I. L., Stadler, P. F., and Schuster, P. (1996b). Analysis of RNA sequence structure maps by exhaustive enumeration II. structures of neutral networks and shape space covering. Chem. Mon., 127, 375-389.
Hofacker, I. L., Fontana, W., Stadler, P. F., Bonhoeffer, L. S., Tacker, M., and Schuster, P. (1994). Fast folding and comparison of RNA secondary structures. Monatsh. Chem., 125, 167-188.
Kimura, M. (1968). Evolutionary rate at the molecular level. Nature, 217, 624-626.
Mathews, D., Sabina, J., Zuker, M., and Turner, D. (1999). Expanded sequence dependence of thermodynamic parameters improves prediction of RNA secondary structure. J. Mol. Biol., 288, 911-940.
Mathews, D., Disney, M., Childs, J., Schroeder, S., Zuker, M., and Turner, D. (2004). Incorporating chemical modification constraints into a dynamic programming algorithm for prediction of RNA secondary structure. Proc Natl Acad Sci, 101, 7287-7292.
McCaskill, J. S. (1990). The equilibrium partition function and base pair binding probabilities for RNA secondary structure. Biopolymers, 29, 1105-1119.
Parisien, M. and Major, F. (2008). The MC-Fold and MC-Sym pipeline infers RNA structure from sequence data. Nature, 452, 51-55.
Reidys, C. M. (1997). Random induced subgraphs of general n-cubes. Adv. Appl. Math., 19, 360-377.
Reidys, C. M. (2002). Distance in random induced subgraphs of generalized n-cubes. Comb, Prob. Comput., 11, 599-605.
Reidys, C. M. (2009). The largest component in random induced subgraphs of n-cubes. Disc. Math., 309(10), 3113-3124.
Reidys, C. M. and Stadler, P. F. (2001). Neutrality in fitness landscapes. Appl. Math. & Comput., 117, 321-350.
Reidys, C. M., Stadler, P. F., and Schuster, P. (1997). Generic properties of combinatory maps and neutral networks of RNA secondary structuresaŁ, Bull. Math. Biol., 59(2), 339-397.
Rogers, E. and Heitsch, C. E. (2014). Profiling small RNA reveals multimodal substructural signals in a boltzmann ensemble. Nucl. Acids Res., 42(22), e171.
Serganov, A. and Patel, D. J. (2007). Ribozymes, riboswitches and beyond: regulation of gene expression without proteins. Nat. Rev. Genet., 10, 776-790.
Tacker, M., Stadler, P. F., Bornberg-Bauer, E. G., Hofacker, I. L., and Schuster, P. (1996). Algorithm independent properties of RNA structure prediction. Eur. Biophy. J., 25, 115-130.
Turner, D. and Mathews, D. H. (2010). NNDB: the nearest neighbor parameter database for predicting stability of nucleic acid secondary structure. Nucl. Acids Res., 38(Database), 280-282.
Waterman, M. S. (1978). Secondary structure of single-stranded nucleic acids. Adv. Math. (Suppl. Studies), 1, 167-212.
Zuker, M. and Stiegler, P. (1981). Optimal computer folding of larger RNA sequences using thermodynamics and auxiliary information. Nucleic Acids Res., 9, 133-148.
Elhanan Borenstein and Eytan Ruppin. Direct evolution of genetic robustness in microrna. Proceedings of the National Academy of Sciences, 103(17):6593-6598, 2006.
Ronald R Breaker. Are engineered proteins getting competition from rna? Current Opinion in Biotechnology, 7(4):442-448, 1996.
Ronald R Breaker and Gerald F Joyce. Inventing and improving ribozyme function: rational design versus iterative selection methods. Trends in biotechnology, 12(7):268-275, 1994.
James E Darnell. RNA: life's indispensable molecule. Cold Spring Harbor Laboratory Press, 2011.
J Arjan G M de Visser, Joachim Hermisson, Gunter P Wagner, Lauren Ancel Meyers, Homayoun Bagheri-Chaichian, Jeffrey L Blanchard, Lin Chao, James M Cheverud, Santiago F Elena, Walter Fontana, et al. Perspective: evolution and detection of genetic robustness. Evolution, 57(9):1959-1972, 2003.
Andreas R Gruber, Stephan H Bernhart, Ivo L Hofacker, and Stefan Washietl. Strategies for measuring evolutionary conservation of rna secondary structures. BMC bioinformatics, 9(1):122, 2008.
Zhenglong Gu, Lars M Steinmetz, Xun Gu, Curt Scharfe, et al. Role of duplicate genes in genetic robustness against null mutations. Nature, 421(6918):63, 2003.
Ronny Lorenz, Stephan H Bernhart, Christian Hoener Zu Siederdissen, Hakim Tafer, Christoph Flamm, Peter F Stadler, and Ivo L Hofacker. Viennarna package 2.0. Algorithms for Molecular Biology, 6(1):26, 2011.
Nicholas Price, Reed A Cartwright, Niv Sabath, Dan Graur, and Ricardo B R Azevedo. Neutral evolution of robustness in drosophila microrna precursors. Molecular biology and evolution, 28(7):2115-2123, 2011.
Guillermo Rodrigo and Mario A Fares. Describing the structural robustness landscape of bacterial small rnas. BMC evolutionary biology, 12(1):52, 2012.
Carl D Schlichting, Massimo Pigliucci, et al. Phenotypic evolution: a reaction norm perspective. Sinauer Associates Incorporated, 1998.
Shi-Jie Chen and Ken A Dill. Rna folding energy landscapes. Proceedings of the National Academy of Sciences, 97(2):646-651, 2000.
Ken A Dill, Hue Sun Chan, et al. From levinthal to pathways to funnels. Nature structural biology, 4(1):10-19, 1997.
Eva Freyhult, Vincent Moulton, and Peter Clote. Boltzmann probability of rna structural neighbors and riboswitch detection. Bioinformatics, 2007.
J. A. Garcia-Martin, A. H. Bayegan, I. Dotu, and P. Clote. RNAdualPF: software to compute the dual partition function with sample applications in molecular evolution theory. BMC Bioinformatics, 17(1):424, 2016.
Ulrike Göbel and Christian V Forst. Rna pathfinder—global properties of neutral networks. Zeitschrift fuer physikalische Chemie, 216(February 2002):175, 2002.
Alex Levin, Mieszko Lis, Yann Ponty, Charles W O?Donnell, Srinivas Devadas, Bonnie Berger, and Jérôme Waldispühl. A global sampling approach to designing and reengineering rna secondary structures. Nucleic acids research, 40(20):10041-10052, 2012.
Ronny Lorenz, Christoph Flamm, and Ivo L Hofacker. 2d projections of rna folding landscapes. GCB, 157(11-20):21, 2009.
Hugo M Martinez. An rna folding rule. 1984.
M. E. Nebel, A. Scheid, and F. Weinberg. Random generation of RNA secondary structures according to native distributions. Algorithms Mol. Biol., 6(24): doi:10.1186/1748-7188-6-24, 2011.
R. Nussinov, G. Piecznik, J. R. Griggs, and D. J. Kleitman. Algorithms for loop matching. SIAM J. Appl. Math., 35(1):68-82, 1978.
José Nelson Onuchic, Zaida Luthey-Schulten, and Peter G Wolynes. Theory of protein folding: the energy landscape perspective. Annual review of physical chemistry, 48(1):545-600, 1997.
Y. Ponty. Efficient sampling of RNA secondary structures from the Boltzmann ensemble of low-energy: the boustrophedon method. J. Math. Biol., 56(1-2):107-27, 2008.
Ignacio Tinoco and Carlos Bustamante. How rna folds. Journal of molecular biology, 293(2):271-281, 1999.
Jérôme Waldispühl, Srinivas Devadas, Bonnie Berger, and Peter Clote. Efficient algorithms for probing the rna mutation landscape. PLoS computational biology, 4(8):e1000124, 2008.
Michael T Wolfinger, W Andreas Svrcek-Seiler, Christoph Flamm, Ivo L Hofacker, and Peter F Stadler. Efficient computation of rna folding dynamics. Journal of Physics A: Mathematical and General, 37(17):4731, 2004.
Sidney Altman. Enzymatic cleavage of rna by ma. Bioscience Reports, 10(4):317-337, 1990.
Thomas R. Cech. Self-splicing and enzymatic activity of an intervening sequence rna from tetrahymena. Bioscience Reports, 10(3):239-261, 1990.
Qingfeng Chen, Gang Li, and Yi-Ping Phoebe Chen. Interval-based distance function for identifying rna structure candidates. Journal of Theoretical Biology, 269(1):280-286, 2011.
J. Cheng, P. Kapranov, J. Drenkow, S. Dike, S. Brubaker, S. Patel, J. Long, D. Stern, H. Tammana, G. Helt, V. Sementchenko, A. Piccolboni, S. Bekiranov, D. K. Bailey, M. Ganesh, S. Ghosh, I. Bell, D. S. Gerhard, and T. R. Gingeras. Transcriptional maps of 10 human chromosomes at 5-nucleotide resolution. Science, 308(5725):1149-54, 2005.
S. R. Eddy. Non-coding RNA genes and the modern rna world. Nat. Rev. Genet., 2(12):919-29, 2001.
Walter Fontana and Peter Schuster. Continuity in evolution: On the nature of transitions. Science, 280(5368):1451-1455, 1998.
W. Grüner, R. Giegerich, D. Strothmann, C. Reidys, J. Weber, I. L. Hofacker, P. F. Stadler, and P. Schuster. Analysis of rna sequence structure maps by exhaustive enumeration i. neutral networks. Monatshefte für Chemie/Chemical Monthly, 127(4):355-374, April 1996.
Christian Haslinger and Peter F. Stadler. RNA structures with pseudo-knots: Graph-theoretical and combinatorial properties. Bull. Math. Biol., 61:437-467, 1999.
I. L. Hofacker. The vienna RNA secondary structure server. Nucl. Acids Res., 31:3429-3431, 2003.
Philipp Kapranov, Jill Cheng, Sujit Dike, David A. Nix, Radharani Duttagupta, Aarron T. Willingham, Peter F. Stadler, Jana Hertel, Jörg Hackermüller, Ivo L. Hofacker, Ian Bell, Evelyn Cheung, Jorg Drenkow, Erica Dumais, Sandeep Patel, Gregg Helt, Madhavan Ganesh, Srinka Ghosh, Antonio Piccolboni, Victor Sementchenko, Hari Tammana, and Thomas R. Gingeras. Rna maps reveal new rna classes and a possible function for pervasive transcription. Science, 316(5830):1484-1488, 2007.
Motoo Kimura. The Neutral Theory of Molecular Evolution. Cambridge University Press, 1983.
Ana Kozomara and Sam Griffiths-Jones. mirbase: annotating high confidence micrornas using deep sequencing data. Nucleic Acids Research, 42(D1):D68-D73, 2014.
Karen A. LeCuyer and Donald M. Crothers. The leptomonas collosoma spliced leader rna can switch between two alternate structural forms. Biochemistry, 32(20):5301-5311, 1993. PMID: 8499434.
Vincent Moulton, Michael Zuker, Michael Steel, Robin Pointon, and David Penny. Metrics on rna secondary structures. Journal of Computational Biology, 7(1-2):277-292, 2004.
Christian Reidys. Combinatorial Computational Biology of RNA. Springer, 2011.
Christian Reidys, Peter F. Stadler, and Peter Schuster. Generic properties of combinatory maps: Neutral networks of rna secondary structures. Bulletin of Mathematical Biology, 59(2):339-397, March 1997.
R. Tyrrell Rockafellar and Wets Roger J.-B. Variational Analysis. Springer, 1998.
P Schuster, W. Fontana, P. F. Stadler, and I. L. Hofacker. From sequences to shapes and back: a case study in RNA secondary structures. Proc Biol Sci., 255(1344):279-84, 1994.
Peter Schuster. How to search for rna structures theoretical concepts in evolutionary biotechnology. Journal of Biotechnology, 41(2):239-257, 1995.
Mimouni, Naila K., Rune B. Lyngsø, Sam Griffiths-Jones, and Jotun Hein. “An analysis of structural influences on selection in RNA genes.” Molecular biology and evolution 26, no. 1 (2008): 209-216.
Pollard, Katherine S., Sofie R. Salama, Nelle Lambert, Marie-Alexandra Lambot, Sandra Coppens, Jakob S. Pedersen, Sol Katzman et al, “An RNA gene expressed during cortical development evolved rapidly in humans,” Nature 443, no. 7108 (2006): 167.
Beniaminov, Artemy, Eric Westhof, and Alain Krol, “Distinctive structures between chimpanzee and humanin a brain noncoding RNA.” RNA 14, no. 7 (2008): 1270-1275.

1. A method of characterizing a nucleic acid sequence, the method comprising:
determining robustness of the nucleic acid sequence based on an inverse fold rate of a sequence structure pair at a predetermined hamming distance associated with the nucleic acid sequence; and
determining plasticity of the genetic sequenced based on base pairing probability.

2. A method of identifying a set of nucleic acid sequences that form desired compatible structures, the method comprising:
generating a first plurality nucleic acid sequences; and
sampling a second plurality of nucleic acid sequences from the first plurality of nucleic acid sequences using a partition function;
wherein the identified set of nucleic acid sequences form the desired compatible structures from the second plurality of nucleic acid sequences; and
wherein the desired compatible structures are functionally equivalent to a specific structure associated with a specific biologic function.

3. The method of claim 2, wherein the generated first plurality of nucleic acid sequences has a predefined energy threshold.

4. The method of claim 2, wherein the identified set includes nucleic acid sequences with progressively differing nucleotide sequences forming the desired compatible structures.

5. The method of claim 2, wherein the desired compatible structures are formed by the identified set at a predefined energy threshold.

6. The method of claim 2, wherein the desired compatible structures formed by the identified set of the nucleic acid sequences are not identical to each other.

7. The method of claim 2, wherein energy states of the desired compatible structures formed by the plurality of nucleic acid structures in the identified set differ.

8. The method of claim 2, wherein the identified set progressively increases in distance from a desired structure, the distance being based on base-pair H-distances.

9. The method of claim 8, wherein the specific structure has a specific nucleotide sequence, and wherein a first nucleic acid sequence in the identified set has a nucleotide sequence more distinguishable from the specific nucleotide sequence than a second nucleic acid sequence in the identified set having a lesser distance from the desired structure.

10. The method of claim 2, wherein at least some of the desired compatible structures are identical.

11. The method of claim 2, wherein the predefined energy threshold is a minimum free energy threshold.

12. A method of identifying a set of nucleic acid sequences that form a specific structure, the method comprising:
generating an initial population of nucleic acid sequences that form the specific structure;
using each of the nucleic acid sequences in the initial population of nucleic acid sequences to generate a first plurality of nucleic acid sequences from each of the nucleic acid sequences in the initial population;
sampling, with a partition function, a second plurality of nucleic acid sequences from the first plurality of nucleic acid sequences that were generated from the initial population of nucleic acid sequences;
wherein the identified set of nucleic acid sequences form the specific structure from the second plurality of nucleic acid sequences sampled from the first plurality of nucleic acid sequences that were generated from the initial population of nucleic acid sequences; and
wherein the specific structure formed by the identified set of nucleic acid sequences is functionally equivalent to a desired specific structure associated with a specific biologic function.

13. The method of claim 12, wherein the specific structure is formed by the generated initial population of nucleic acid sequences that are identical.

14. The method of claim 12, wherein the specific structure is formed by the generated initial population of nucleic acid sequences formed at a predefined energy threshold.

15. The method of claim 12, wherein the generated first plurality of nucleic acid sequences has a predefined energy threshold.

16. The method of claim 12, wherein the identified set includes nucleic acid sequences with progressively differing nucleotide sequences forming the specific structure.

17. The method of claim 12, wherein the specific structures formed by the identified set of the nucleic acid sequences are not identical to each other.

18. The method of claim 12, wherein energy states of the specific structures formed by the plurality of nucleic acid structures in the identified set differ.

19. The method of claim 12, wherein the identified set progressively increases in distance from the specific structure, the distance being based on base-pair H-distances.

20. The method of claim 12, wherein the specific structure has a specific nucleotide sequence, and wherein a first nucleic acid sequence in the identified set has a nucleotide sequence more distinguishable from the specific nucleotide sequence than a second nucleic acid sequence in the identified set having a lesser distance from the desired structure.
